(19) United States
(12) Patent Application Publication
(10) Pub. No.: US 2024/0366690 A1
(43) Pub. Date: Nov. 7, 2024

(54) MICROBIOME TREATMENTS FOR ANIMAL FERTILITY

(71) Applicant: VYTELLE LLC, Lenexa, KS (US)

(72) Inventors: Tasha Gruhot, Hawarden, IA (US); Bruno Valente Sanches, Southlake, TX (US)

(21) Appl. No.: 18/626,627

(22) Filed: Apr. 4, 2024

Related U.S. Application Data
(60) Provisional application No. 63/494,139, filed on Apr. 4, 2023.

(51) Int. Cl. A61K 35/742; A61K 35/745; A61K 35/747; C12Q 1/689; G16H 50/30

(52) U.S. Cl. CPC A61K 35/742; A61K 35/745; A61K 35/747; C12Q 1/689; G16H 50/30; C12Q 2600/106

(57) ABSTRACT

The present disclosure provides, inter alia, methods for increasing fertility in a bovine animal, which methods include obtaining a microbiome profile for a bovine animal, quantifying the abundance of individual bacterial species, and determining whether the bovine animal exhibits a microbiome profile that is predictive of successful embryo implantation and, thereby, assessing the likelihood of a successful in vitro fertilization (IVF) derived embryo implantation, and pregnancy. In further aspects, the present disclosure provides microbiota supplementation treatment(s) to improve bovine fertility, and/or diagnostic test kits that are predictive of successful or unsuccessful embryo implantation and maintenance of pregnancy. Also provided are compositions and kits for use in those methods to obtain and quantify the microbiome profile of a bovine animal.

### CROSS REFERENCE TO RELATED APPLICATIONS

This application claims priority under 35 U.S.C. § 119 to provisional patent application U.S. Ser. No. 63/494,139, filed Apr. 4, 2023. The provisional patent application is herein incorporated by reference in their entirety, including without limitation, the specification, claims, and abstract, as well as any figures, tables, appendices, or drawings thereof.

### FIELD OF THE INVENTION

The present disclosure relates, generally, to diagnostic technologies and methodologies for assessing the fertility of a candidate bovine surrogate dam and for improving the success rate of both embryo implantation and maintenance of pregnancy to full term.

### BACKGROUND OF THE INVENTION

In vitro fertilization (IVF) and embryo transfer techniques are commonly used in bovine calf production. IVF has some advantages over other production methods due to the frequency with which IVF aspirations can be performed and the ability to increase genetic diversity. For example, donor cow IVF aspirations can be performed every two weeks and semen from several different bulls can be used to fertilize harvested oocytes to produce a large number of offspring from a single cow. While traditional in vivo embryo flushing techniques can produce about five or six embryos per collection every sixty days, IVF collections can produce about 20 oocytes per aspiration every two weeks, of which about 30% develop into viable embryos. In some circumstances, IVF can be used to produce over 50 calves from one cow in a single year. Furthermore, pregnant donor cows can still be used for oocyte collection until about day 100 to day 120 of pregnancy, making it possible to produce embryos while still producing natural offspring from a high value cow.

There are, however, some disadvantages with using IVF techniques for ruminant offspring production. The IVF procedure can be expensive and the follicle aspiration method used to collect oocytes is an invasive procedure that requires a skilled technician. Furthermore, even when performed properly, freshly transferred IVF embryos result in an average pregnancy rate of about 50% for well managed recipient bovine surrogate dams. Of the transfers that result in pregnancy, between about 6% to about 16% of pregnancies are lost.

Existing methodologies for identifying a surrogate dam that is a suitable candidate for embryo transfer is the presence of a Corpus Luteum at time of transfer. Thus, despite recent advancements in IVF techniques, there remains an unmet need in the art for improved technologies and methodologies for maximizing the efficiency of offspring production, including diagnostic technologies and methodologies for assessing the fertility of a candidate recipient bovine surrogate dam and for improving the success rate of both embryo implantation and maintenance of pregnancy to full term.

### GLOSSARY

Unless defined otherwise, all technical and scientific terms used above have the same meaning as commonly understood by one of ordinary skill in the art to which embodiments of the present invention pertain.

The terms “a,” “an,” and “the” include both singular and plural referents.

The term “or” is synonymous with “and/or” and means any one member or combination of members of a particular list.

The terms “invention” or “present invention” are not intended to refer to any single embodiment of the particular invention but encompass all possible embodiments as described in the specification and the claims.

The term “about” as used herein refer to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variable, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “scope” of the present invention is defined by the appended claims, along with the full scope of equivalents to which such claims are entitled. The scope of the invention is further qualified as including any possible modification to any of the aspects and/or embodiments disclosed herein which would result in other embodiments, combinations, subcombinations, or the like that would be obvious to those skilled in the art.

The terms “in vitro fertilization” and “IVF” are used interchangeably to refer to IVF is a type of assisted reproductive technology wherein oocytes collected from a donor cow are fertilized in vitro, and the resulting embryos are implanted (single embryo implant) into the uterus of a recipient (surrogate) dam that maintains a pregnancy through to birth.

The term “biomarker” refers to a measured characteristic that is used as an indicator of a biological state or condition. In the present disclosure, “biomarkers” include “microbiome biomarkers,” in particular “bacterial biomarkers,” which are used diagnostically in the methodology disclosed herein for predicting IVF embryo transfer efficiency, including assessing the suitability of a bovine surrogate dam for embryo implantation as well as for maintaining a pregnancy to full term. The diagnostic methodology for quantifying bacterial biomarker levels and ratios in the microbiome of vaginal and uterine fluids from candidate recipient bovine surrogate dams, which is disclosed herein, exhibit both good sensitivity (thereby correctly identify high fertility surrogate dams) as well as good specificity (thereby correctly identify low fertility surrogate dams).

The term “treatment” refers to interventions that may be used advantageously to increase the success rate of embryo transfer.

The “scope” of the present disclosure is defined by the appended claims, along with the full scope of equivalents to which such claims are entitled. The scope of the invention is further qualified as including any possible modification to any of the aspects and/or embodiments disclosed herein which would result in other embodiments, combinations, subcombinations, or the like that would be obvious to those skilled in the art.

Where a term is provided in the singular, other embodiments described by the plural of that term are also provided. As used herein, the terms “include,” “includes,” and “including” are to be construed as at least having the features to which they refer while not excluding any additional unspecified features. It will be understood that, unless indicated to the contrary, terms intended to be “open” (e.g., the term “including” should be interpreted as “including but not limited to,” the term “having” should be interpreted as “having at least,” the term “includes” should be interpreted as “includes but is not limited to,” etc.). Phrases such as “at least one,” and “one or more,” and terms such as “a” or “an” include both the singular and the plural.

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “configured” describes structure capable of performing a task or adopting a particular configuration. The term “configured” can be used interchangeably with other similar phrases, such as constructed, arranged, adapted, manufactured, and the like. Terms characterizing sequential order, a position, and/or an orientation are not limiting and are only referenced according to the views presented.

It will be further understood that where features or aspects of the disclosure are described in terms of Markush groups, the disclosure is also intended to be described in terms of any individual member or subgroup of members of the Markush group. Similarly, all ranges disclosed herein also encompass all possible sub-ranges and combinations of sub-ranges and that language such as “between,” “up to,” “at least,” “greater than,” “less than,” and the like include the number recited in the range and includes each individual member.

The term “cattle” refers to one or more animals of the Bos taurus or Bos indicus species.

The term “beef cattle” refers to cattle for breeding and/or use in beef production.

The term “dairy cattle” refers to cattle for breeding and/or use in dairy production.

The term “fertility” means reproductive capacity or capability.

All references cited herein, whether supra or infra, including, but not limited to, patents, patent applications, and patent publications, whether U.S., PCT, or non-U.S. foreign, and all technical and/or scientific publications are hereby incorporated by reference in their entirety.

Unless defined otherwise, all technical and scientific terms used above have the same meaning as commonly understood by one of ordinary skill in the art to which embodiments of the present invention pertain.

### BRIEF DESCRIPTION OF THE DRAWINGS

FIG. 1 Principal Coordinate Analysis (PCoA) of bacteria by breed type and pregnancy status post embryo transfer based on bacteria membership.

FIG. 2 illustrates Principal Coordinate Analysis (PCoA) of the bacteria membership within dairy recipients based on pregnancy status post embryo transfer.

FIG. 3 illustrates Principal Coordinate Analysis (PCoA) of the bacteria membership within beef recipients based on pregnancy status post embryo transfer.

FIG. 4 illustrates Principal Coordinate Analysis (PCoA) of bacteria by breed type and pregnancy status post embryo transfer based on bacteria structure.

FIG. 5 illustrates Principal Coordinate Analysis (PCoA) of bacteria structure in dairy recipients based on pregnancy status post embryo transfer.

FIG. 6 illustrates Principal Coordinate Analysis (PCoA) of bacteria structure in beef recipients based on pregnancy status post embryo transfer.

FIG. 7 illustrates a Multidimensional Scaling (MDS) of microbiota structure in beef and dairy recipients based on pregnancy status post embryo transfer using machine learning analysis (Euclidean Distance).

FIG. 8 indicates bacteria with an LDA cut-off of 3 between beef recipients based on pregnancy status post embryo transfer.

FIG. 9 is graphical representation of the significant difference in relative abundance of the bacteria Sphingomandaceae unclassified in pregnant versus non-pregnant beef recipients after embryo transfer.

FIG. 10 is graphical representation of the significant difference in relative abundance of Corynebacterium the in pregnant versus non-pregnant beef recipients after embryo transfer.

FIG. 11 is graphical representation of the significant difference in relative abundance of Arcanobacterium the in pregnant versus non-pregnant beef recipients after embryo transfer.

FIG. 12 indicates bacteria with an LDA cut-off of 4 between dairy recipients based on pregnancy status post embryo transfer.

FIG. 13 is graphical representation of the significant difference in relative abundance of Lachnospiraceae unclassified the in pregnant versus non-pregnant dairy recipients after embryo transfer.

FIG. 14 is graphical representation of the significant difference in relative abundance of Histophilus the in pregnant versus non-pregnant dairy recipients after embryo transfer.

FIG. 15 is graphical representation of the significant difference in relative abundance of Fusobacteriaceae unclassified the in pregnant versus non-pregnant dairy recipients after embryo transfer.

FIG. 16 is graphical representation of the significant difference in relative abundance of Treponema the in pregnant versus non-pregnant dairy recipients after embryo transfer.

FIG. 17 is graphical representation of the significant difference in relative abundance of Pelistega the in pregnant versus non-pregnant dairy recipients after embryo transfer.

FIG. 18 is graphical representation of the significant difference in relative abundance of Canibacter the in pregnant versus non-pregnant dairy recipients after embryo transfer.

FIG. 19 is graphical representation of the significant difference in relative abundance of Erysipelotricheae unclassified the in pregnant versus non-pregnant dairy recipients after embryo transfer.

FIG. 20 is graphical representation of the significant difference in relative abundance of Bifidobacterium the in pregnant versus non-pregnant dairy recipients after embryo transfer.

FIG. 21 is graphical representation of the significant difference in relative abundance of Alcaligenaceae unclassified the in pregnant versus non-pregnant dairy recipients after embryo transfer.

FIG. 22 is graphical representation of the significant difference in relative abundance of Gammaproteabacteria unclassified the in pregnant versus non-pregnant dairy recipients after embryo transfer.

FIG. 23 is graphical representation of the significant difference in relative abundance of Coriobacteria unclassified the in pregnant versus non-pregnant dairy recipients after embryo transfer.

FIG. 24 is graphical representation of the significant difference in relative abundance of Frisingicoccus the in pregnant versus non-pregnant dairy recipients after embryo transfer.

FIG. 25 is graphical representation of the significant difference in relative abundance of Ruminobacter the in pregnant versus non-pregnant dairy recipients after embryo transfer.

FIG. 26 is graphical representation of the significant difference in relative abundance of Gemella the in pregnant versus non-pregnant dairy recipients after embryo transfer.

FIG. 27 is graphical representation of the significant difference in relative abundance of Prevotella the in pregnant versus non-pregnant dairy recipients after embryo transfer.

FIG. 28 is graphical representation of the significant difference in relative abundance of Anaerobutyricum the in pregnant versus non-pregnant dairy recipients after embryo transfer.

FIG. 29 is graphical representation of the significant difference in relative abundance of Marinitenerispora the in pregnant versus non-pregnant dairy recipients after embryo transfer.

FIG. 30 is graphical representation of the significant difference in relative abundance of Parvimonas the in pregnant versus non-pregnant dairy recipients after embryo transfer.

FIG. 31 is graphical representation of the significant difference in relative abundance of Agathobacter the in pregnant versus non-pregnant dairy recipients after embryo transfer.

FIG. 32 is graphical representation of the significant difference in relative abundance of Olsenella the in pregnant versus non-pregnant dairy recipients after embryo transfer.

FIG. 33 is graphical representation of the significant difference in relative abundance of Ligilactobacullus the in pregnant versus non-pregnant dairy recipients after embryo transfer.

FIG. 34 is graphical representation of the significant difference in relative abundance of Psychrobacillus the in pregnant versus non-pregnant dairy recipients after embryo transfer.

FIG. 35 is graphical representation of the significant difference in relative abundance of Victivallis the in pregnant versus non-pregnant dairy recipients after embryo transfer.

FIG. 36 is graphical representation of the significant difference in relative abundance of Vulgatibacter the in pregnant versus non-pregnant dairy recipients after embryo transfer.

FIG. 37 is graphical representation of the significant difference in relative abundance of Irregularibacter the in pregnant versus non-pregnant dairy recipients after embryo transfer.

FIG. 38 is graphical representation of the significant difference in relative abundance of Cumulibacter the in pregnant versus non-pregnant dairy recipients after embryo transfer.

FIG. 39 is graphical representation of the significant difference in relative abundance of Micrococcales unclassified the in pregnant versus non-pregnant dairy recipients after embryo transfer.

FIG. 40 is graphical representation of the significant difference in relative abundance of Oscillibacter the in pregnant versus non-pregnant dairy recipients after embryo transfer.

FIG. 41 is graphical representation of the significant difference in relative abundance of Anaerotignum the in pregnant versus non-pregnant dairy recipients after embryo transfer.

FIG. 42 is graphical representation of the significant difference in relative abundance of Rikenellaceae Unclassified the in pregnant versus non-pregnant dairy recipients after embryo transfer.

FIG. 43 is graphical representation of the significant difference in relative abundance of Verrucomicrobiales Unclassified the in pregnant versus non-pregnant dairy recipients after embryo transfer.

FIG. 44 is graphical representation of the significant difference in relative abundance of Cohnella the in pregnant versus non-pregnant dairy recipients after embryo transfer.

FIG. 45 is graphical representation of the significant difference in relative abundance of Monoglobus the in pregnant versus non-pregnant dairy recipients after embryo transfer.

FIG. 46 is graphical representation of the significant difference in relative abundance of Clostridia Unclassified the in pregnant versus non-pregnant dairy recipients after embryo transfer.

FIG. 47 is graphical representation of the significant difference in relative abundance of Akkermansia the in pregnant versus non-pregnant dairy recipients after embryo transfer.

FIG. 48 is graphical representation of the significant difference in relative abundance of Ihubacter the in pregnant versus non-pregnant dairy recipients after embryo transfer.

FIG. 49 is graphical representation of the significant difference in relative abundance of Muribaculaceae Unclassified the in pregnant versus non-pregnant dairy recipients after embryo transfer.

FIG. 50 is graphical representation of the significant difference in relative abundance of Mediterranea the in pregnant versus non-pregnant dairy recipients after embryo transfer.

FIG. 51 is graphical representation of the significant difference in relative abundance of Alistipes the in pregnant versus non-pregnant dairy recipients after embryo transfer.

FIG. 52 is graphical representation of the significant difference in relative abundance of Firmicutes Unclassified the in pregnant versus non-pregnant dairy recipients after embryo transfer.

FIG. 53 is graphical representation of the significant difference in relative abundance of Ruminococcaceae Unclassified the in pregnant versus non-pregnant dairy recipients after embryo transfer.

FIG. 54 is graphical representation of the significant difference in relative abundance of Bacteria Unclassified the in pregnant versus non-pregnant dairy recipients after embryo transfer.

FIG. 55 is graphical representation of the significant difference in relative abundance of Bacteroidetes Unclassified the in pregnant versus non-pregnant dairy recipients after embryo transfer.

FIG. 56 indicates bacteria with an LDA cut-off of 4 between recipients (regardless of breed type) based on pregnancy status post embryo transfer.

FIG. 57 is graphical representation of the significant difference in relative abundance of Lachnospiraceae Unclassified the in pregnant versus non-pregnant (regardless of breed type) recipients after embryo transfer.

FIG. 58 is graphical representation of the significant difference in relative abundance of Fusobacteriaceae Unclassified the in pregnant versus non-pregnant (regardless of breed type) recipients after embryo transfer.

FIG. 59 is graphical representation of the significant difference in relative abundance of Pelistega the in pregnant versus non-pregnant (regardless of breed type) recipients after embryo transfer.

FIG. 60 is graphical representation of the significant difference in relative abundance of Erysipelotrichaceae Unclassified the in pregnant versus non-pregnant (regardless of breed type) recipients after embryo transfer.

FIG. 61 is graphical representation of the significant difference in relative abundance of Bifidobacterium the in pregnant versus non-pregnant (regardless of breed type) recipients after embryo transfer.

FIG. 62 is graphical representation of the significant difference in relative abundance of Bifidobacteriaceae Unclassified the in pregnant versus non-pregnant (regardless of breed type) recipients after embryo transfer.

FIG. 63 is graphical representation of the significant difference in relative abundance of Sphingomonadaceae Unclassified the in pregnant versus non-pregnant (regardless of breed type) recipients after embryo transfer.

FIG. 64 is graphical representation of the significant difference in relative abundance of Fusobacterium the in pregnant versus non-pregnant (regardless of breed type) recipients after embryo transfer.

FIG. 65 is graphical representation of the significant difference in relative abundance of Alcaligenaceae Unclassified the in pregnant versus non-pregnant (regardless of breed type) recipients after embryo transfer.

FIG. 66 is graphical representation of the significant difference in relative abundance of Gammaproteobacteria Unclassified the in pregnant versus non-pregnant (regardless of breed type) recipients after embryo transfer.

FIG. 67 is graphical representation of the significant difference in relative abundance of Frisingincoccus the in pregnant versus non-pregnant (regardless of breed type) recipients after embryo transfer.

FIG. 68 is graphical representation of the significant difference in relative abundance of Parvimonas the in pregnant versus non-pregnant (regardless of breed type) recipients after embryo transfer.

FIG. 69 is graphical representation of the significant difference in relative abundance of Prevotella the in pregnant versus non-pregnant (regardless of breed type) recipients after embryo transfer.

FIG. 70 is graphical representation of the significant difference in relative abundance of Azohydromonas the in pregnant versus non-pregnant (regardless of breed type) recipients after embryo transfer.

FIG. 71 is graphical representation of the significant difference in relative abundance of Anaerobutyricum the in pregnant versus non-pregnant (regardless of breed type) recipients after embryo transfer.

FIG. 72 is graphical representation of the significant difference in relative abundance of Bacteroidetes Unclassified the in pregnant versus non-pregnant (regardless of breed type) recipients after embryo transfer.

FIG. 73 is graphical representation of the significant difference in relative abundance of Bacteria Unclassified the in pregnant versus non-pregnant (regardless of breed type) recipients after embryo transfer.

FIG. 74 is graphical representation of the significant difference in relative abundance of Alistipes the in pregnant versus non-pregnant (regardless of breed type) recipients after embryo transfer.

FIG. 75 is graphical representation of the significant difference in relative abundance of Mediterranea the in pregnant versus non-pregnant (regardless of breed type) recipients after embryo transfer.

FIG. 76 is graphical representation of the significant difference in relative abundance of Ihubacter the in pregnant versus non-pregnant (regardless of breed type) recipients after embryo transfer.

FIG. 77 is graphical representation of the significant difference in relative abundance of Akkermansia the in pregnant versus non-pregnant (regardless of breed type) recipients after embryo transfer.

FIG. 78 is graphical representation of the significant difference in relative abundance of Monoglobus the in pregnant versus non-pregnant (regardless of breed type) recipients after embryo transfer.

FIG. 79 is graphical representation of the significant difference in relative abundance of Clostridia Unclassified the in pregnant versus non-pregnant (regardless of breed type) recipients after embryo transfer.

FIG. 80 is graphical representation of the significant difference in relative abundance of Rikenellaceae Unclassified the in pregnant versus non-pregnant (regardless of breed type) recipients after embryo transfer.

FIG. 81 is graphical representation of the significant difference in relative abundance of Pseudorhodoferax the in pregnant versus non-pregnant (regardless of breed type) recipients after embryo transfer.

FIG. 82 is graphical representation of the significant difference in relative abundance of Victivallis the in pregnant versus non-pregnant (regardless of breed type) recipients after embryo transfer.

FIG. 83 is a table listing microbiota that had at least a 15% point difference in incidence (presence in particular sampled female) when comparing all pregnant versus open recipients post embryo transfer, with the higher incidence of microbiota in females that got pregnant from the embryo transfer.

FIGS. 84A, 84B, and 84C are a table listing microbiota that were present in at least 85% of females that became pregnant from the embryo transfer: There were 76 microbiota that were found in at least 85% of pregnant dairy recipients, and there were 72 microbiota that were found in at least 85% of pregnant beef recipients.

FIG. 85 is a table listing microbiota that had at least a 15% point difference in incidence (presence in particular sampled female) when comparing all pregnant versus open recipients post embryo transfer. with the higher incidence of microbiota in females that did not get pregnant from the embryo transfer.

FIGS. 86A and 86B represent a random forest machine learning model used to select for features of importance (microbiota) to predict recipient pregnancy status. Presented are the features of importance for predicting pregnancy status in Beef recipients.

FIGS. 87A and 87B represent a random forest machine learning model used to select for features of importance (microbiota) to predict recipient pregnancy status. Presented are the features of importance for predicting pregnancy status in Dairy recipients.

FIG. 88 is a random forest machine learning model was used to select for features of importance (microbiota) to predict recipient pregnancy status. Blinded samples not included in the training set were used to test the prediction models, the confusion matrix is presented showing a 60% accuracy in predicting pregnancy status post embryo transfer using selected microbiota features of importance from the model.

### SUMMARY OF THE INVENTION

The present disclosure fulfills unmet needs in the art by providing methods, potential microbial supplementation treatments to positively influence the vagina and uterus making the environment more suitable to embryo pregnancy as well as associated diagnostic test kits for determining the suitability of candidate bovine animals to serve as surrogate dams as recipients for in vitro fertilization (IVF), including their ability or inability to establish a successful embryo implantation and to maintain a successful pregnancy. The methods, proposed microbiota treatments for vaginal supplementation and diagnostic test kits disclosed herein employ vaginal microbiome composition, presence and abundance from a bovine surrogate dam that are predictive of successful or unsuccessful embryo implantation and maintenance of pregnancy, which methods, microbiota supplementation treatment(s) and/or diagnostic test kits overcome limitations in the art by permitting the identification of a suitable surrogate dam for embryo transfer at the time of or prior to the day of embryo transfer or supplementing the disclosed bacteria found to be associated with pregnancy at embryo transfer. The methods, microbiota supplementation and/or and diagnostic test kits disclosed herein can increase the selection accuracy of candidate surrogate dams and increase the potential of pregnancy in supplemented dams and result in higher overall pregnancy rates. The methods include administering an intervention to the recipient bovine surrogate dam, wherein the intervention is effective to provide the recipient bovine surrogate dam with a microbiome profile associated with a high level of embryo implantation and maintenance of pregnancy.

Within certain embodiments, provided herein are methods for predicting in a candidate bovine surrogate dam an ability or inability to establish a successful embryo implantation and pregnancy following embryo transfer. In certain aspects, these methods comprise (1) obtaining vaginal fluids from a candidate bovine surrogate dam, wherein said vaginal fluids comprises a vaginal microbiome and (2) performing microbiome analysis on the vaginal fluids to obtain a vaginal bacterial profile comprising two or more vaginal bacterial species. According to these embodiments, the microbiome profile of a candidate bovine surrogate dam is predictive of a candidate bovine surrogate dam possessing a suitable or an unsuitable environment for achieving a successful embryo implantation and maintaining a successful pregnancy. Also provided are methods for supplementing favorable bacteria into the vaginal increase odds of pregnancy following embryo transfer. In certain aspects, these methods comprise incorporation of microbiota into a gel or paste to be administered to the animal as a vaginal suppository.

Within other embodiments, provided herein are methods for generating a microbiome database for identifying a bovine surrogate dam for embryo transfer. In certain aspects, these methods comprise (1) collecting vaginal fluids individually from a population of bovine animals, wherein the vaginal fluids comprise a microbiome of each bovine animal of the population, (2) performing microbiome analysis on the vaginal fluids to obtain a vaginal bacterial profile, wherein the vaginal microbiome profile is predictive of a bovine surrogate dam possessing a suitable or an unsuitable environment for achieving a successful embryo implantation and maintaining a successful pregnancy, and (4) identifying an association of vaginal bacterial species identification and/or levels in the vaginal microbiome profile with an increased probability of a successful embryo implantation and pregnancy following embryo delivery.

Within further embodiments, provided herein are methods for improving the success rate of in vitro fertilization embryo transfer in bovine animals serving as surrogate dams. In certain aspects, these methods comprise (1) collecting vaginal fluid from each of the bovine animals, wherein the vaginal fluid comprises a microbiome of each bovine animal; (2) performing microbiome analysis on the vaginal fluids to obtain a vaginal bacterial profile, wherein the vaginal bacterial profile is predictive of bovine animals possessing or not possessing an environment for achieving a successful embryo implantation and pregnancy following in vitro fertilization and therefore being suitable or unsuitable bovine animals for serving as surrogate dams; and (3) administering an intervention to one or more of the bovine surrogate dams, wherein the intervention is effective to provide or augment in a recipient bovine surrogate dam a microbiome profile that is associated with a high likelihood of successful in vitro fertilization, including reliable embryo implantation and maintenance of pregnancy.

Within still further embodiments, provided herein are diagnostic test kits for use in the identification of bovine surrogate dams having a high and/or a low probability of establishing a successful embryo implantation and maintaining pregnancy following embryo transfer via in vitro fertilization. In certain aspects, diagnostic test kits comprise: (1) one or more analytical tools for determining a vaginal microbiome profile comprising vaginal bacterial species and/or levels from a bovine animal; (2) a transmitter for communicating with and/or connecting to a database of one or more vaginal bacterial species and/or levels of a bovine vaginal fluid; (3) a device for comparing the vaginal bacterial species and/or levels from candidate bovine surrogate dams with vaginal bacterial species and/or levels in a database of vaginal microbiomes to identify a bovine surrogate dam having a vaginal microbiome that is predictive of a high or a low success rate of embryo implantation and maintaining pregnancy after receiving an in vitro fertilization derived embryo; and, optionally, (4) instructions for the use of the diagnostic test kits. Diagnostic test kits according to these embodiments may employ microbiota sequencing techniques and instruments to allow field or onsite collection of vaginal fluids and subsequent microbiome analysis to establish bacterial species and/or ratios that convey a positive or negative relationship to the maternal recognition of pregnancy and intrauterine embryo implantation.

These and other related aspects of the present disclosure will be better understood in light of the following drawings and detailed description, which exemplify various embodiments, and which are presented for illustration, not limitation.

### EMBODIMENTS

The following numbered embodiments form part of the present disclosure.
1. A method of increasing fertility in cattle comprising administering a microbiome supplement to the cattle comprising a combination of bacteria selected from the group consisting of Aerococcaceae_unclassified, planomicrobium, Staphylococcaceae_unclassified, Clostridia_unclassified, Bacteroidales_unclassified, Micrococcaceae_unclassified, paludibacter, brevundimonas, Dietzia, Staphylococcus, Burkholderiales_unclassified, Saccharofermentans, solibacillus, Sporobacter, cystobacteraceae_unclassified, Porphyromonadaceae_unclassified, Longibaculum, Micomonosporaceae_unclassified, Weeksellaceae_unclassified, Geobacillus, Mogibacterium, Actinobacteria_unclassified, Trueperella, Colidextribacter, microvirga, pseudactionotalea, Sphingosinicellaceae_unclassified, Firmicutes_unclassified, Comamonadaceae_unclassified, Paradevosia, Peptoniphilus, Candidatus_Solibacter_unclassified, Neochlamydia, Ruminococcaceae_unclassified, Flaviflexus, Gp3_unclassified, Ramlibacter, Bacteroides, Acetobacteraceae_unclassified, Micrococcales_unclassified, Cytophagales_unclassified, Victivallis, Ohtaekwangia, Parcubacteria_unclassified, Ruminococcus, Erysipelotrichales_unclassified, Planctomycetacia_unclassified, Bifidobacteriaceae_unclassified, Alistipes, Eggerthellaceae_unclassified, Truepera, Roseimaritima, Nocardiaceae_unclassified, Myroides, Flintibacter, Devosia, Limosilactobacillus, Ornithinibacillus, Porphyromonas, Anaerolineaceae_unclassified, Streptococcaceae_unclassified, Faecalibacterium, Bacteroidaceae_unclassified, Verrucomicrobia_unclassified, Caldilineaceae_unclassified, Cellvibrio, Ornithinimicrobium, Gemmatimonadaceae_unclassified, Erysipelotrichaceae_unclassified, Ureaplasma, Xanthomonadaceae_unclassified, Jeotgalicoccus, Verrucomicrobiales_unclassified, Methylobacterium, Monoglobus, Spirochaetaceae_unclassified, Ruminobacter, Paenibacillaceae_1_unclassified, Rhizobiales_unclassified, Neisseriaceae_unclassified, Tumebacillus, Flavobacteriales_unclassified, Bacillaceae_2_unclassified, Petroclostridium, Atopobiaceae_unclassified, Eubacteriaceae_unclassified, Tissierellaceae_unclassified, Mediterranea, Phyllobacterium, Coriobacteriales_unclassified, Raoultibacter, Sphaerobacteraceae_unclassified, Roseburia, Butyricicoccus, Ruminobacter, Oscillibacter, Pseudonocardiaceae_unclassified, Nocardiopsaceae_unclassified, Flavonifractor, Anaerolineae_unclassified, Bacillales_unclassified, Spirochaetaceae_unclassified, Treponema, Paraeggerthella, Monoglobus, Ureaplasma, Succinivibrio, Cellulosilyticum, Fusobacterium, Bacilli_unclassified, Paramuribaculum, Streptococcaceae_unclassified, Subdivision5_unclassified, Planococcaceae_unclassified, Campylobacter, Thermoactinomyces, Micrococcales_unclassified, Alistipes, Aminipila, Paenibacillaceae_1_unclassified, Facklamia, Saccharomonospora, Vaginisenegalia, Nocardiaceae_unclassified, Hespellia, Anaerotignum, Streptococcus, Ruminococcus, Paludibacter, Odoribacter, Devosiaceae_unclassified, Muribaculum, Bacteroidaceae_unclassified, Thermoactinomycetaceae_1_unclassified, Atopostipes, Micrococcaceae_unclassified, Gammaproteobacteria_unclassified, Holdemania, Victivallis, Vulcaniibacterium, Nocardioidaceae_unclassified, Stenotrophomonas, Aquihabitans, Trueperella, Flavobacteriales_unclassified, Helcococcus, Mucinivorans, Weissella, Opitutae_unclassified, Harryflintia, Moraxella, Parvimonas, Sutterella, Kroppenstedtia, Pseudescherichia, Lachnobacterium, Jeotgalicoccus, Kandleria, Longicatena, Succiniclasticum, Terrisporobacter, Ornithinimicrobium, Parabacteroides, Stackebrandtia, Peptoniphilus, Bacillaceae_1_unclassified, Anaerobacillus, Alkalihalobacillus, Caldibacillus, Sphingobacteriaceae_unclassified, Crocinitomix, Membranicola, Cryomorpha, Tumebacillus, Effusibacillus, Alicyclobacillaceae_unclassified, Fibrobacter, Fibrobacteres_unclassified, Aeribacillus, Sphingobacterium, Falsibacillus, Chitinispirillum, and Saprospiraceae_unclassified.

2. A method of increasing fertility in cattle comprising administering a microbiome supplement to the cattle comprising a combination of bacteria selected from the group consisting of Anaerobutyricum, Azohydromonas, Prevotella, Parvimonas, Frisingincoccus, Gammaproteobacteria Unclassified, Alcaligenaceae Unclassified, Fusobacterium, Sphingomonadaceae Unclassified, Bifidobacteriaceae Unclassified, Bifidobacterium, Erysipelotrichaceae Unclassified, Pelistega, Fusobacteriaceae Unclassified, and Lachnospiraceae Unclassified.

3. A method of increasing fertility in beef cattle comprising administering a microbiome supplement to the cattle comprising a combination of bacteria selected from the group consisting of Aerococcaceae_unclassified, planomicrobium, Staphylococcaceae_unclassified, Clostridia_unclassified, Bacteroidales_unclassified, Micrococcaceae_unclassified, paludibacter, brevundimonas, Dietzia, Staphylococcus, Burkholderiales_unclassified, Saccharofermentans, solibacillus, Sporobacter, cystobacteraceae_unclassified, Porphyromonadaceae_unclassified, Longibaculum, Micomonosporaceae_unclassified, Weeksellaceae_unclassified, Geobacillus, Mogibacterium, Actinobacteria_unclassified, Trueperella, Colidextribacter, microvirga, pseudactionotalea, Sphingosinicellaceae_unclassified, Firmicutes_unclassified, Comamonadaceae_unclassified, Paradevosia, Peptoniphilus, Candidatus_Solibacter_unclassified, Neochlamydia, Ruminococcaceae_unclassified, Flaviflexus, Gp3_unclassified, Ramlibacter, Bacteroides, Acetobacteraceae_unclassified, Micrococcales_unclassified, Cytophagales_unclassified, Victivallis, Ohtaekwangia, Parcubacteria_unclassified, Ruminococcus, Erysipelotrichales_unclassified, Planctomycetacia_unclassified, Bifidobacteriaceae_unclassified, Alistipes, Eggerthellaceae_unclassified, Truepera, Roseimaritima, Nocardiaceae_unclassified, Myroides, Flintibacter, Devosia, Limosilactobacillus, Ornithinibacillus, Porphyromonas, Anaerolineaceae_unclassified, Streptococcaceae_unclassified, Faecalibacterium, Bacteroidaceae_unclassified, Verrucomicrobia_unclassified, Caldilineaceae_unclassified, Cellvibrio, Ornithinimicrobium, Gemmatimonadaceae_unclassified, Erysipelotrichaceae_unclassified, Ureaplasma, Xanthomonadaceae_unclassified, Jeotgalicoccus, Verrucomicrobiales_unclassified, Methylobacterium, Monoglobus, Spirochaetaceae_unclassified, Ruminobacter, Paenibacillaceae_1_unclassified, Rhizobiales_unclassified, Neisseriaceae_unclassified, Tumebacillus, Flavobacteriales_unclassified, Bacillaceae_2_unclassified, and Petroclostridium.

4. A method of increasing fertility in beef cattle comprising administering a microbiome supplement to the cattle comprising a combination of bacteria selected from the group consisting of Sphingomonadaceae unclassified, Corynebacterium, Staphylococcus, Streptococcus, Staphylococcaceae unclassified, Bogoriellaceae unclassified, Saccharomonospora, Bdellovibrio, Brevibacterium, Carnobacteriaceae unclassified, Succiniclasticum, Actinobacteria unclassified, and Sneathia.

5. The method of embodiment 4, wherein the bacteria comprise Corynebacterium or Sphingomonadaceae unclassified.

6. A method of increasing fertility in dairy cattle comprising administering a microbiome supplement to the cattle comprising a combination of bacteria selected from the group consisting of Atopobiaceae_unclassified, Eubacteriaceae_unclassified, Tissierellaceae_unclassified, Mediterranea, Phyllobacterium, Coriobacteriales_unclassified, Raoultibacter, Sphaerobacteraceae_unclassified, Roseburia, Butyricicoccus, Ruminobacter, Oscillibacter, Pseudonocardiaceae_unclassified, Nocardiopsaceae_unclassified, Flavonifractor, Anaerolineae_unclassified, Bacillales_unclassified, Spirochaetaceae_unclassified, Treponema, Paraeggerthella, Monoglobus, Ureaplasma, Succinivibrio, Cellulosilyticum, Fusobacterium, Bacilli_unclassified, Paramuribaculum, Streptococcaceae_unclassified, Subdivision5_unclassified, Planococcaceae_unclassified, Campylobacter, Thermoactinomyces, Micrococcales_unclassified, Alistipes, Aminipila, Paenibacillaceae_1_unclassified, Facklamia, Saccharomonospora, Vaginisenegalia, Nocardiaceae_unclassified, Hespellia, Anaerotignum, Streptococcus, Ruminococcus, Paludibacter, Odoribacter, Devosiaceae_unclassified, Muribaculum, Bacteroidaceae_unclassified, Thermoactinomycetaceae_1_unclassified, Atopostipes, Micrococcaceae_unclassified, Gammaproteobacteria_unclassified, Holdemania, Victivallis, Vulcaniibacterium, Nocardioidaceae_unclassified, Stenotrophomonas, Aquihabitans, Trueperella, Flavobacteriales_unclassified, Helcococcus, Mucinivorans, Weissella, Opitutae_unclassified, Harryflintia, Moraxella, Parvimonas, Sutterella, Kroppenstedtia, Pseudescherichia, Lachnobacterium, Kandleria, Longicatena, Succiniclasticum, Terrisporobacter, Ornithinimicrobium, Parabacteroides, Stackebrandtia, Peptoniphilus, Bacillaceae_1_unclassified, Anaerobacillus, Alkalihalobacillus, Caldibacillus, Sphingobacteriaceae_unclassified, Crocinitomix, Membranicola, Cryomorpha, Tumebacillus, Effusibacillus, Alicyclobacillaceae_unclassified, Fibrobacter, Fibrobacteres_unclassified, Aeribacillus, Sphingobacterium, Falsibacillus, Chitinispirillum, and Saprospiraceae_unclassified.

7. A method of increasing fertility in dairy cattle comprising administering a microbiome supplement to the cattle comprising a combination of bacteria selected from the group consisting of Lachnospiraceae unclassified, Histophilus, Stomatobactulum, Rothia, Bifidobacteriaceae unclassified, Fusobacterium, Quasibacillus, Limosilactobacillus, Fusicatenibacter, Paraeggerthella, Anaeroplasma, Mogibacterium, Neisseriaceae unclassified, Fusobacteriacea unclassified, Canibacter, Erysipelotrichaceae unclassified, Pelistega, Gemella, Prevotella, Parvimonas, Ruminobacter, Gammaproteobacteria unclassified, Bifidobacterium, Selenomonadaceae unclassified, Prevotellaceae unclassified, Alcaligenaceae unclassified, Salinicoccus, Gp16 unclassified, Camelimonas, Schwartzia, Aeromucrobium, Marvinbryantia, Desulfovibrianaceae unclassified, Lachnobacterium, Pasteurellaceae unclassified, Mycobacteriales unclassified, Micrococcaceae unclassified, Weissella, Treponema, Coriobacteriia unclassified, Frisingicoccus, Anaerobutyricum, Marinitenerispora, Agathobacter, Olsenella, and Ligilactobacillus.

8. The method of embodiment 7, wherein the bacteria comprise Lachnospiraceae unclassified, Histophilus, Fusobacteriacea unclassified, Canibacter, Erysipelotrichaceae unclassified, Pelistega, Gemella, Prevotella, Parvimonas, Ruminobacter, Gammaproteobacteria unclassified, Bifidobacterium, Alcaligenaceae unclassified, Treponema, Coriobacteriia unclassified, Frisingicoccus, Anaerobutyricum, Marinitenerispora, Agathobacter, Olsenella, or Ligilactobacillus.

9. A microbiome supplement for increasing fertility in cattle comprising a combination of bacteria selected from the group consisting of Aerococcaceae_unclassified, planomicrobium, Staphylococcaceae_unclassified, Clostridia_unclassified, Bacteroidales_unclassified, Micrococcaceae_unclassified, paludibacter, brevundimonas, Dietzia, Staphylococcus, Burkholderiales_unclassified, Saccharofermentans, solibacillus, Sporobacter, cystobacteraceae_unclassified, Porphyromonadaceae_unclassified, Longibaculum, Micomonosporaceae_unclassified, Weeksellaceae_unclassified, Geobacillus, Mogibacterium, Actinobacteria_unclassified, Trueperella, Colidextribacter, microvirga, pseudactionotalea, Sphingosinicellaceae_unclassified, Firmicutes_unclassified, Comamonadaceae_unclassified, Paradevosia, Peptoniphilus, Candidatus_Solibacter_unclassified, Neochlamydia, Ruminococcaceae_unclassified, Flaviflexus, Gp3_unclassified, Ramlibacter, Bacteroides, Acetobacteraceae_unclassified, Micrococcales_unclassified, Cytophagales_unclassified, Victivallis, Ohtaekwangia, Parcubacteria unclassified, Ruminococcus, Erysipelotrichales_unclassified, Planctomycetacia_unclassified, Bifidobacteriaceae_unclassified, Alistipes, Eggerthellaceae_unclassified, Truepera, Roseimaritima, Nocardiaceae_unclassified, Myroides, Flintibacter, Devosia, Limosilactobacillus, Ornithinibacillus, Porphyromonas, Anaerolineaceae_unclassified, Streptococcaceae_unclassified, Faecalibacterium, Bacteroidaceae_unclassified, Verrucomicrobia_unclassified, Caldilineaceae_unclassified, Cellvibrio, Ornithinimicrobium, Gemmatimonadaceae_unclassified, Xanthomonadaceae_unclassified, Methylobacterium, Monoglobus, Erysipelotrichaceae_unclassified, Ureaplasma, Jeotgalicoccus, Verrucomicrobiales_unclassified, Spirochaetaceae_unclassified, Ruminobacter, Paenibacillaceae_1_unclassified, Rhizobiales_unclassified, Neisseriaceae_unclassified, Tumebacillus, Flavobacteriales_unclassified, Bacillaceae_2_unclassified, Petroclostridium, Atopobiaceae_unclassified, Eubacteriaceae_unclassified, Tissierellaceae_unclassified, Mediterranea, Phyllobacterium, Coriobacteriales_unclassified, Raoultibacter, Sphaerobacteraceae_unclassified, Roseburia, Butyricicoccus, Ruminobacter, Oscillibacter, Pseudonocardiaceae_unclassified, Nocardiopsaceae_unclassified, Flavonifractor, Anaerolineae_unclassified, Bacillales_unclassified, Spirochaetaceae_unclassified, Treponema, Paraeggerthella, Monoglobus, Ureaplasma, Succinivibrio, Cellulosilyticum, Fusobacterium, Bacilli_unclassified, Paramuribaculum, Streptococcaceae_unclassified, Subdivision5_unclassified, Planococcaceae_unclassified, Campylobacter, Thermoactinomyces, Micrococcales_unclassified, Alistipes, Aminipila, Paenibacillaceae_1_unclassified, Facklamia, Saccharomonospora, Vaginisenegalia, Nocardiaceae_unclassified, Hespellia, Anaerotignum, Streptococcus, Ruminococcus, Paludibacter, Odoribacter, Devosiaceae_unclassified, Muribaculum, Bacteroidaceae_unclassified, Thermoactinomycetaceae_1_unclassified, Atopostipes, Micrococcaceae_unclassified, Gammaproteobacteria_unclassified, Holdemania, Victivallis, Vulcaniibacterium, Nocardioidaceae_unclassified, Stenotrophomonas, Aquihabitans, Trueperella, Flavobacteriales_unclassified, Helcococcus, Mucinivorans, Weissella, Opitutae_unclassified, Harryflintia, Moraxella, Parvimonas, Sutterella, Kroppenstedtia, Pseudescherichia, Lachnobacterium, Jeotgalicoccus, Kandleria, Longicatena, Succiniclasticum, Terrisporobacter, Ornithinimicrobium, Parabacteroides, Stackebrandtia, Peptoniphilus, Bacillaceae_1_unclassified, Anaerobacillus, Alkalihalobacillus, Caldibacillus, Sphingobacteriaceae_unclassified, Crocinitomix, Membranicola, Cryomorpha, Tumebacillus, Effusibacillus, Alicyclobacillaceae_unclassified, Fibrobacter, Fibrobacteres_unclassified, Aeribacillus, Sphingobacterium, Falsibacillus, Chitinispirillum, and Saprospiraceae_unclassified.

10. A microbiome supplement for increasing fertility in beef cattle comprising a combination of bacteria selected from the group consisting of Aerococcaceae_unclassified, planomicrobium, Staphylococcaceae_unclassified, Clostridia_unclassified, Bacteroidales_unclassified, Micrococcaceae_unclassified, paludibacter, brevundimonas, Dietzia, Staphylococcus, Burkholderiales_unclassified, Saccharofermentans, solibacillus, Sporobacter, cystobacteraceae_unclassified, Porphyromonadaceae_unclassified, Longibaculum, Micomonosporaceae_unclassified, Weeksellaceae_unclassified, Geobacillus, Mogibacterium, Actinobacteria_unclassified, Trueperella, Colidextribacter, microvirga, pseudactionotalea, Sphingosinicellaceae_unclassified, Firmicutes_unclassified, Comamonadaceae_unclassified, Paradevosia, Peptoniphilus, Candidatus_Solibacter_unclassified, Neochlamydia, Ruminococcaceae_unclassified, Flaviflexus, Gp3_unclassified, Ramlibacter, Bacteroides, Acetobacteraceae_unclassified, Micrococcales_unclassified, Cytophagales_unclassified, Victivallis, Ohtaekwangia, Parcubacteria_unclassified, Ruminococcus, Erysipelotrichales_unclassified, Planctomycetacia_unclassified, Bifidobacteriaceae_unclassified, Alistipes, Eggerthellaceae_unclassified, Truepera, Roseimaritima, Nocardiaceae_unclassified, Myroides, Flintibacter, Devosia, Limosilactobacillus, Ornithinibacillus, Porphyromonas, Anaerolineaceae_unclassified, Streptococcaceae_unclassified, Faecalibacterium, Bacteroidaceae_unclassified, Verrucomicrobia_unclassified, Caldilineaceae_unclassified, Cellvibrio, Ornithinimicrobium, Gemmatimonadaceae_unclassified, Erysipelotrichaceae_unclassified, Ureaplasma, Xanthomonadaceae_unclassified, Jeotgalicoccus, Verrucomicrobiales_unclassified, Methylobacterium, Monoglobus, Spirochaetaceae_unclassified, Ruminobacter, Paenibacillaceae_1_unclassified, Rhizobiales_unclassified, Neisseriaceae_unclassified, Tumebacillus, Flavobacteriales_unclassified, Bacillaceae_2_unclassified, and Petroclostridium.

11. A microbiome supplement for increasing fertility in beef cattle comprising a combination of bacteria selected from the group consisting of Sphingomonadaceae unclassified, Corynebacterium, Staphylococcus, Streptococcus, Staphylococcaceae unclassified, Bogoriellaceae unclassified, Saccharomonospora, Bdellovibrio, Brevibacterium, Carnobacteriaceae unclassified, Succiniclasticum, Actinobacteria unclassified, and Sneathia.

12. The supplement of embodiment 11, wherein the bacteria are Corynebacterium and/or Sphingomandaceae.

13. A microbiome supplement for increasing fertility in dairy cattle comprising a combination of bacteria selected from the group consisting of Atopobiaceae_unclassified, Eubacteriaceae_unclassified, Tissierellaceae_unclassified, Mediterranea, Phyllobacterium, Coriobacteriales_unclassified, Raoultibacter, Sphaerobacteraceae_unclassified, Roseburia, Butyricicoccus, Ruminobacter, Oscillibacter, Pseudonocardiaceae_unclassified, Nocardiopsaceae_unclassified, Flavonifractor, Anaerolineae_unclassified, Bacillales_unclassified, Spirochaetaceae_unclassified, Treponema, Paraeggerthella, Monoglobus, Ureaplasma, Succinivibrio, Cellulosilyticum, Fusobacterium, Bacilli_unclassified, Paramuribaculum, Streptococcaceae_unclassified, Subdivision5_unclassified, Planococcaceae_unclassified, Campylobacter, Thermoactinomyces, Micrococcales_unclassified, Alistipes, Aminipila, Paenibacillaceae_1_unclassified, Facklamia, Saccharomonospora, Vaginisenegalia, Nocardiaceae_unclassified, Hespellia, Anaerotignum, Streptococcus, Ruminococcus, Paludibacter, Odoribacter, Devosiaceae_unclassified, Muribaculum, Bacteroidaceae_unclassified, Thermoactinomycetaceae_1_unclassified, Atopostipes, Micrococcaceae_unclassified, Gammaproteobacteria_unclassified, Holdemania, Victivallis, Vulcaniibacterium, Nocardioidaceae_unclassified, Stenotrophomonas, Aquihabitans, Trueperella, Flavobacteriales_unclassified, Helcococcus, Mucinivorans, Weissella, Opitutae_unclassified, Harryflintia, Moraxella, Parvimonas, Sutterella, Kroppenstedtia, Pseudescherichia, Lachnobacterium, Jeotgalicoccus, Kandleria, Longicatena, Succiniclasticum, Terrisporobacter, Ornithinimicrobium, Parabacteroides, Stackebrandtia, Peptoniphilus, Bacillaceae_1_unclassified, Anaerobacillus, Alkalihalobacillus, Caldibacillus, Sphingobacteriaceae_unclassified, Crocinitomix, Membranicola, Cryomorpha, Tumebacillus, Effusibacillus, Alicyclobacillaceae_unclassified, Fibrobacter, Fibrobacteres_unclassified, Aeribacillus, Sphingobacterium, Falsibacillus, Chitinispirillum, and Saprospiraceae_unclassified.

14. A microbiome supplement for increasing fertility in dairy cattle comprising a combination of bacteria selected from the group consisting of Lachnospiraceae unclassified, Histophilus, Stomatobactulum, Rothia, Bifidobacteriaceae unclassified, Fusobacterium, Quasibacillus, Limosilactobacillus, Fusicatenibacter, Paraeggerthella, Anaeroplasma, Mogibacterium, Neisseriaceae unclassified, Fusobacteriacea unclassified, Canibacter, Erysipelotrichaceae unclassified, Pelistega, Gemella, Prevotella, Parvimonas, Ruminobacter, Gammaproteobacteria unclassified, Bifidobacterium, Selenomonadaceae unclassified, Prevotellaceae unclassified, Alcaligenaceae unclassified, Salinicoccus, Gp16 unclassified, Camelimonas, Schwartzia, Aeromucrobium, Marvinbryantia, Desulfovibrianaceae unclassified, Lachnobacterium, Pasteurellaceae unclassified, Mycobacteriales unclassified, Micrococcaceae unclassified, Weissella, Treponema, Coriobacteriia unclassified, Frisingicoccus, Anaerobutyricum, Marinitenerispora, Agathobacter, Olsenella, and Ligilactobacillus.

15. The microbiome supplement of claim 14, wherein the bacteria comprise Ligilactobacullus, Olsenella, Agathobacter, Parvimonas, Marinitenerispora, Anaerobutyricum, Prevotella, Gemella, Ruminobacter, Frisingicoccus, Coriobacteria, Gammaproteabacteria unclassified, Alcaligenaceae, Bifidobacterium, Erysipelotricheae, Canibacter, Pelistega, Treponema, Fusobacteriaceae, Histophilus, or Lachnospiraceae unclassified.

16. The supplement of embodiments 9, 10, 11, 13, or 14 wherein the supplement is administered orally.

17. The supplement of embodiments 9, 10, 11, 13, or 14 wherein the supplement is administered vaginally.

18. The method of embodiments 1, 3, or 4 wherein the breed is selected from the group consisting of Angus, Hereford, Simmental, Charolais, Red Angus, Brahman.

19. The methods of embodiments 1, 6 or 7 wherein the breed is selected from the group consisting of Holstein, Jersey, Swedish Red, or Mt. Boulliard.
(a) A method of increasing fertility in cattle comprising administering a microbiome supplement comprising a combination of bacteria selected from the group consisting of Aerococcaceae_unclassified, planomicrobium, Staphylococcaceae_unclassified, Clostridia_unclassified, Bacteroidales_unclassified, Micrococcaceae_unclassified, paludibacter, brevundimonas, Dietzia, Staphylococcus, Burkholderiales_unclassified, Saccharofermentans, solibacillus, Sporobacter, cystobacteraceae_unclassified, Porphyromonadaceae_unclassified, Longibaculum, Micomonosporaceae_unclassified, Weeksellaceae_unclassified, Geobacillus, Mogibacterium, Actinobacteria_unclassified, Trueperella, Colidextribacter, microvirga, pseudactionotalea, Sphingosinicellaceae_unclassified, Firmicutes_unclassified, Comamonadaceae_unclassified, Paradevosia, Peptoniphilus, Candidatus_Solibacter_unclassified, Neochlamydia, Ruminococcaceae_unclassified, Flaviflexus, Gp3_unclassified, Ramlibacter, Bacteroides, Acetobacteraceae_unclassified, Micrococcales_unclassified, Cytophagales_unclassified, Victivallis, Ohtaekwangia, Parcubacteria_unclassified, Ruminococcus, Erysipelotrichales_unclassified, Planctomycetacia_unclassified, Bifidobacteriaceae_unclassified, Alistipes, Eggerthellaceae_unclassified, Truepera, Roseimaritima, Nocardiaceae_unclassified, Myroides, Flintibacter, Devosia, Limosilactobacillus, Ornithinibacillus, Porphyromonas, Anaerolineaceae_unclassified, Streptococcaceae_unclassified, Faecalibacterium, Bacteroidaceae_unclassified, Verrucomicrobia_unclassified, Caldilineaceae_unclassified, Cellvibrio, Ornithinimicrobium, Gemmatimonadaceae_unclassified, Erysipelotrichaceae_unclassified, Ureaplasma, Xanthomonadaceae_unclassified, Jeotgalicoccus, Verrucomicrobiales_unclassified, Methylobacterium, Monoglobus, Spirochaetaceae_unclassified, Ruminobacter, Paenibacillaceae_1_unclassified, Rhizobiales_unclassified, Neisseriaceae unclassified, Tumebacillus, Flavobacteriales_unclassified, Bacillaceae_2_unclassified, Petroclostridium, Atopobiaceae_unclassified, Eubacteriaceae_unclassified, Tissierellaceae_unclassified, Mediterranea, Phyllobacterium, Coriobacteriales_unclassified, Raoultibacter, Sphaerobacteraceae_unclassified, Roseburia, Butyricicoccus, Ruminobacter, Oscillibacter, Pseudonocardiaceae_unclassified, Nocardiopsaceae_unclassified, Flavonifractor, Anaerolineae_unclassified, Bacillales_unclassified, Spirochaetaceae_unclassified, Treponema, Paraeggerthella, Monoglobus, Ureaplasma, Succinivibrio, Cellulosilyticum, Fusobacterium, Bacilli_unclassified, Paramuribaculum, Streptococcaceae_unclassified, Subdivision5_unclassified, Planococcaceae_unclassified, Campylobacter, Thermoactinomyces, Micrococcales_unclassified, Alistipes, Aminipila, Paenibacillaceae_1_unclassified, Facklamia, Saccharomonospora, Vaginisenegalia, Nocardiaceae_unclassified, Hespellia, Anaerotignum, Streptococcus, Ruminococcus, Paludibacter, Odoribacter, Devosiaceae_unclassified, Muribaculum, Bacteroidaceae_unclassified, Thermoactinomycetaceae_1_unclassified, Atopostipes, Micrococcaceae_unclassified, Gammaproteobacteria_unclassified, Holdemania, Victivallis, Vulcaniibacterium, Nocardioidaceae_unclassified, Stenotrophomonas, Aquihabitans, Trueperella, Flavobacteriales_unclassified, Helcococcus, Mucinivorans, Weissella, Opitutae_unclassified, Harryflintia, Moraxella, Parvimonas, Sutterella, Kroppenstedtia, Pseudescherichia, Lachnobacterium, Jeotgalicoccus, Kandleria, Longicatena, Succiniclasticum, Terrisporobacter, Ornithinimicrobium, Parabacteroides, Stackebrandtia, Peptoniphilus, Bacillaceae_1 unclassified, Anaerobacillus, Alkalihalobacillus, Caldibacillus, Sphingobacteriaceae_unclassified, Crocinitomix, Membranicola, Cryomorpha, Tumebacillus, Effusibacillus, Alicyclobacillaceae_unclassified, Fibrobacter, Fibrobacteres_unclassified, Aeribacillus, Sphingobacterium, Falsibacillus, Chitinispirillum, and Saprospiraceae_unclassified to the cattle,
(b) measuring the level of fertility in the animal before and after administering the supplement, and;
(c) determining an increase in fertility in the animal as a result of administering the supplement.

20. A method for improving fertility in one or more bovine animals, the method comprising:

(a) collecting vaginal fluid from each of the bovine animals, wherein the vaginal fluid comprises a microbiome of each the bovine animal; (b) performing microbiome analysis on the vaginal fluids to obtain a vaginal microbiome profile, wherein the microbiome composition is predictive of an increased fertility; and (c) administering an intervention to one or more of the bovine dams, wherein the intervention is effective to provide a bovine animal with a microbiome profile associated with an increased fertility.

21. A diagnostic test kit for identifying a bovine animal having increased fertility comprising:
(a) one or more analytical tools for determining a vaginal microbiome profile comprising bacterial species from the bovine animal; (b) a transmitter to communicate/connect a database of one or more microbiome components of the vaginal fluid; (c) a device for comparing the vaginal microbiome from the bovine animal with a database of vaginal microbiome which identifies a bovine animal having a vaginal microbiome that is predictive of increased fertility; and (d) and instructions for use.

22. A diagnostic test kit for identifying a bovine animal having decreased fertility comprising:
(a) one or more analytical tools for determining a vaginal microbiome profile comprising bacterial species from the bovine animal; (b) a transmitter to communicate/connect a database of one or more microbiome components of the vaginal fluid; (c) a device for comparing the vaginal microbiome from the bovine animal with a database of vaginal microbiome which identifies a bovine animal having a vaginal microbiome that is predictive of decreased fertility; and (d) and instructions for use.

23. A method of increasing fertility in cattle comprising administering an antimicrobial to the cattle targeted to bacteria selected from the group comprising Falcatimonas, Dorea, Pelagibacterium, Lawsonibacterium, Clostridium IV, Planococcaceae unclassified, Sporobacter, Rhodothermales unclassified, Vulcaniibacterium, Rikenellaceae unclassified, Anaerobacter, Alkalibacterium, Harryfintia, Verrucomicrobiales unclassified, Nocardiopsis, Ihubacter, Oscillibacter, Victivallis, Actinomarinicola, Akkermansia, Alistipes, Bacteroidaceae unclassified, Bacteroides unclassified, Bacillales unclassified, Streptosporangiaceae unclassified, Mediterranea, Lactobacillies unclassified, Turicibacter, Hazenalla, Tissierella, Roseburia, Deinococci unclassified, Gulosibacter, Schaedlerella, Psychrobacillus, Vulgatibacter, Irregularibacter, Cumulibacter, Micrococcales unclassified, Anaerotignum, Cohnella, Clostridia unclassified, Muribaculaceae unclassified, Firmicutes unclassified, Rumunococcaceae unclassified, Arcanobacterium, Flintibacter, Leptotrichia, Succinivibrio, Fusobacteriales inclassified, Treponema, Lactobacillus, Patulibacter, Pseudarthrobacter, Acidbacter unclassified, Sandaracinus, Pseudorhodoferax and Histophilus.

24. A method of increasing fertility in cattle comprising administering an antimicrobial to the cattle targeted to bacteria selected from the group comprising Victivallis, Pseudorhodoferax, Rikenellaceae Unclassified, Clostridia Unclassified, Monoglobus, Akkermansia, Ihubacter, Mediterranea, Alistipes, and Bacteroidetes Unclassified.

25. A method of increasing fertility in beef cattle, comprising administering an antimicrobial to the beef cattle targeted to bacteria selected from the group comprising Arcanobacterium, Flintibacter, Leptotrichia, Succinivibrio, Fusobacteriales inclassified, Treponema, Lactobacillus, Patulibacter, Pseudarthrobacter, Acidbacter unclassified, Sandaracinus, and Histophilus.

26. The method of embodiment 26, wherein the bacteria is Arcanobacterium.

27. A method of increasing fertility in dairy cattle, comprising administering an antimicrobial to the dairy cattle targeted to bacteria selected from the group comprising Falcatimonas, Dorea, Pelagibacterium, Lawsonibacterium, Clostridium IV, Planococcaceae unclassified, Sporobacter, Rhodothermales unclassified, Vulcaniibacterium, Rikenellaceae unclassified, Anaerobacter, Alkalibacterium, Harryfintia, Verrucomicrobiales unclassified, Nocardiopsis, Ihubacter, Oscillibacter, Victivallis, Actinomarinicola, Akkermansia, Alistipes, Bacteroidaceae unclassified, Bacteroides unclassified, Bacillales unclassified, Streptosporangiaceae unclassified, Mediterranea, Lactobacill unclassified, Turicibacter, Hazenalla, Tissierella, Roseburia, Deinococci unclassified, Gulosibacter, Schaedlerella, Psychrobacillus, Vulgatibacter, Irregularibacter, Cumulibacter, Micrococcales unclassified, Anaerotignum, Cohnella, Clostridia unclassified, Muribaculaceae unclassified, Firmicutes unclassified, and Rumunococcaceae unclassified.

28. A method of increasing fertility in dairy cattle, comprising administering an antimicrobial to the dairy cattle targeted to bacteria selected from the group comprising Bacteroidetes Unclassified, Bacteria Unclassified, Ruminococcaceae Unclassified, Firmicutes Unclassified, Alistipes, Mediterranea, Muribaculaceae Unclassified, Ihubacter, Akkermansia, Clostridia Unclassified, Monoglobus, Cohnella, Verrucomicrobiales Unclassified, Rikenellaceae Unclassified, Anaerotignum, Oscillibacter, Micrococcales unclassified, Cumulibacter, Irregularibacter, Vulgatibacter, Victivallis, and Psychrobacillus.

29. A supplement for increasing fertility in cattle comprising an antimicrobial for use in cattle which decreases the population of bacteria selected from the group comprising Falcatimonas, Dorea, Pelagibacterium, Lawsonibacterium, Clostridium IV, Planococcaceae unclassified, Sporobacter, Rhodothermales unclassified, Vulcaniibacterium, Rikenellaceae unclassified, Anaerobacter, Alkalibacterium, Harryfintia, Verrucomicrobiales unclassified, Nocardiopsis, Ihubacter, Oscillibacter, Victivallis, Actinomarinicola, Akkermansia, Alistipes, Bacteroidaceae unclassified, Bacteroides unclassified, Bacillales unclassified, Streptosporangiaceae unclassified, Mediterranea, Lactobacillies unclassified, Turicibacter, Hazenalla, Tissierella, Roseburia, Deinococci unclassified, Gulosibacter, Schaedlerella, Psychrobacillus, Vulgatibacter, Irregularibacter, Cumulibacter, Micrococcales unclassified, Anaerotignum, Cohnella, Clostridia unclassified, Muribaculaceae unclassified, Firmicutes unclassified, Rumunococcaceae unclassified, Arcanobacterium, Flintibacter, Leptotrichia, Succinivibrio, Fusobacteriales inclassified, Treponema, Lactobacillus, Patulibacter, Pseudarthrobacter, Acidbacter unclassified, Sandaracinus, Pseudorhodoferax and Histophilus.

30. A supplement for increasing fertility in cattle comprising an antimicrobial for use in cattle which decreases the population of bacteria selected from the group comprising Victivallis, Pseudorhodoferax, Rikenellaceae Unclassified, Clostridia Unclassified, Monoglobus, Akkermansia, Ihubacter, Mediterranea, Alistipes, and Bacteroidetes Unclassified.

31. A supplement for increasing fertility in beef cattle comprising an antimicrobial for use in beef cattle which decreases the population of bacteria selected from the group comprising Arcanobacterium, Flintibacter, Leptotrichia, Succinivibrio, Fusobacteriales inclassified, Treponema, Lactobacillus, Patulibacter, Pseudarthrobacter, Acidbacter unclassified, Sandaracinus, and Histophilus.

32. The supplement of embodiment 32 wherein the bacteria to be decreased is Arcanobacterium.

33. A supplement for increasing fertility in dairy cattle comprising an antimicrobial for use in dairy cattle which decreases the population of bacteria selected from the group comprising Falcatimonas, Dorea, Pelagibacterium, Lawsonibacterium, Clostridium IV, Planococcaceae unclassified, Sporobacter, Rhodothermales unclassified, Vulcaniibacterium, Rikenellaceae unclassified, Anaerobacter, Alkalibacterium, Harryfintia, Verrucomicrobiales unclassified, Nocardiopsis, Ihubacter, Oscillibacter, Victivallis, Actinomarinicola, Akkermansia, Alistipes, Bacteroidaceae unclassified, Bacteroides unclassified, Bacillales unclassified, Streptosporangiaceae unclassified, Mediterranea, Lactobacillus classified, Turicibacter, Hazenalla, Tissierella, Roseburia, Deinococci unclassified, Gulosibacter, Schaedlerella, Psychrobacillus, Vulgatibacter, Irregularibacter, Cumulibacter, Micrococcales unclassified, Anaerotignum, Cohnella, Clostridia unclassified, Muribaculaceae unclassified, Firmicutes unclassified, Monoglobus, and Rumunococcaceae unclassified.

34. A supplement for increasing fertility in dairy cattle comprising an antimicrobial for use in dairy cattle which decreases the population of bacteria selected from the group comprising Rikenellaceae unclassified, Verrucomicrobiales unclassified, Ihubacter, Oscillibacter, Victivallis, Akkermansia, Alistipes, Bacteroides unclassified, Mediterranea, Psychrobacillus, Vulgatibacter, Irregularibacter, Cumulibacter, Micrococcales unclassified, Anaerotignum, Clostridia unclassified, Muribaculaceae unclassified, Firmicutes unclassified, Monoglobus, and Rumunococcaceae unclassified.

35. The supplement of embodiments 30, 31, 32, 34, or 35, wherein the supplement is administered orally.

36. The supplement of embodiments 30, 31, 32, 34, or 35, wherein the supplement is administered vaginally.

37. While various embodiments have been disclosed herein, other embodiments will be apparent to those skilled in the art. The various embodiments disclosed herein are for purposes of illustration and are not intended to be limiting, with the true scope and spirit being indicated by the claims. Moreover, the embodiments described in this disclosure can be combined in various ways. Any aspect or feature that is described for one embodiment can be incorporated into any other embodiment mentioned in this disclosure. While various aspects of presently disclosed embodiments are described herein, it will be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation. The disclosures of all patent and scientific literature cited herein are expressly incorporated by reference in their entirety.

### DETAILED DESCRIPTION OF THE INVENTION

The present disclosure provides methods and diagnostic test kits for (1) the identification of bovine animals that exhibit a low probability of successful embryo implantation and maintaining pregnancy and, thus, are unsuitable candidates to act as surrogate dams for embryo transfer and (2) the selection of bovine animals that exhibit a high probability of embryo implantation and maintaining pregnancy and, thus, are suitable candidates to act as surrogate dams for embryo transfer. According to some aspects, the present disclosure provides methods utilizing a database of microbiome profiles from vaginal fluids for the selection of bovine surrogate dams and, thereby, to improve IVF efficiency by increasing the likelihood of embryo implantation and maintenance of successful pregnancies.

Also provided are methods for supplementing favorable bacteria into the vaginal increase odds of pregnancy following embryo transfer. In certain aspects, these methods comprise incorporation of microbiota into a gel or paste to be administered to the animal as a vaginal suppository.

According to some aspects, the present disclosure provides a method for identifying a recipient bovine surrogate dam for embryo transfer comprising obtaining samples containing the vagina or uterus microbiome. In some embodiments, the method further comprises identifying a microbiome profile for the vaginal or uterine fluid samples from the recipient bovine surrogate dam. In some embodiments, the microbiome profiles are generated by identifying bacterial species and quantifying bacterial levels in fluid samples.

According to some embodiments, the microbiome profiles obtained from the recipient bovine surrogate dam are compared to a database of microbiota profiles. In some embodiments, if the microbiome profile of the recipient bovine surrogate dam is associated with a high rate of initiation of pregnancy and high maintenance of pregnancy to term, then one or more oocytes or ovum are obtained from a donor subject, fertilized by in vitro fertilization, and implanted into the recipient subject. In some embodiments, the vaginal and uterine microbiome profiles of the recipient bovine surrogate dam are indicative of high success of pregnancy after embryo transferred to the female recipient subject.

In some embodiments, the microbiome profile of the recipient bovine surrogate dam indicates a probability of embryo implantation and/or maintenance of pregnancy of greater than 50%. In some embodiments, the microbiome profile of the recipient bovine surrogate dam indicates a probability of embryo implantation and/or maintenance of pregnancy of greater than 55%. In some embodiments, the microbiome profile of the recipient bovine surrogate dam indicates a probability of embryo implantation and/or maintenance of pregnancy of greater than 60%. In some embodiments, the microbiome profile of the recipient bovine surrogate dam indicates a probability of embryo implantation and/or maintenance of pregnancy of greater than 65%. In some embodiments, the microbiome profile of the recipient bovine surrogate dam indicates a probability of embryo implantation and/or maintenance of pregnancy of greater than 70%. In some embodiments, the microbiome profile of the recipient bovine surrogate dam indicates a probability of embryo implantation and/or maintenance of pregnancy of greater than 75%. In some embodiments, the microbiome profile of the recipient bovine surrogate dam indicates a probability of embryo implantation and/or maintenance of pregnancy of greater than 80%. In some embodiments, the microbiome profile of the recipient bovine surrogate dam indicates a probability of embryo implantation and/or maintenance of pregnancy of greater than 85%. In some embodiments, the microbiome profile of the recipient bovine surrogate dam indicates a probability of embryo implantation and/or maintenance of pregnancy of greater than 90%. In some embodiments, the microbiome profile of the recipient bovine surrogate dam indicates a probability of embryo implantation and/or maintenance of pregnancy of greater than 95%. In some embodiments, the microbiome profile of the recipient bovine surrogate dam indicates a probability of embryo implantation and/or maintenance of pregnancy of greater than 99%.

In other embodiments, the microbiome profile of the recipient bovine surrogate dam indicates a probability of embryo implantation and/or maintenance of pregnancy of less than 50%. In some embodiments, the microbiome profile of the recipient bovine surrogate dam indicates a probability of embryo implantation and/or maintenance of pregnancy of less than 45%. In some embodiments, the microbiome profile of the recipient bovine surrogate dam indicates a probability of embryo implantation and/or maintenance of pregnancy of less than 40%. In some embodiments, the microbiome profile of the recipient bovine surrogate dam indicates a probability of embryo implantation and/or maintenance of pregnancy of less than 35%. In some embodiments, the microbiome profile of the recipient bovine surrogate dam indicates a probability of embryo implantation and/or maintenance of pregnancy of less than 30%. In some embodiments, the microbiome profile of the recipient bovine surrogate dam indicates a probability of embryo implantation and/or maintenance of pregnancy of less than 25%. In some embodiments, the microbiome profile of the recipient bovine surrogate dam indicates a probability of embryo implantation and/or maintenance of pregnancy of less than 20%. In some embodiments, the microbiome profile of the recipient bovine surrogate dam indicates a probability of embryo implantation and/or maintenance of pregnancy of less than 15%. In some embodiments, the microbiome profile of the recipient bovine surrogate dam indicates a probability of embryo implantation and/or maintenance of pregnancy of less than 10%. In some embodiments, the microbiome profile of the recipient bovine surrogate dam indicates a probability of embryo implantation and/or maintenance of pregnancy of less than 5%. In some embodiments, the microbiome profile of the recipient bovine surrogate dam indicates a probability of embryo implantation and/or maintenance of pregnancy of less than 1%.

In certain embodiments, the present disclosure provides methods for generating a microbiome database for identifying a recipient bovine surrogate dam to receive an embryo created via in vitro fertilization. In certain embodiments, the present disclosure provides methods for generating a microbiome database for identifying a recipient bovine surrogate dam to receive an embryo created in vivo produced embryos.

In some aspects of these embodiments, the database is a collection of microbiome profile information that is organized so that it can be easily accessed, updated, and compared to other profiles, such as a computer database. In other aspects, the microbiome information in the database is obtained by collecting fluid samples from a population of recipient bovine surrogate dams, wherein the samples are obtained from one or more of the vaginal and/or uterine fluid. In further aspects, the bacterial species information from the fluid samples and is organized into a microbiome profile.

Methods for generating a microbiome database for identifying a recipient bovine surrogate dam may further comprise identifying a microbiome profile for one or more of the vaginal and/or uterine fluid samples from each subject of a population of recipient bovine surrogate dams.

Methods for generating a microbiome database for identifying a recipient bovine surrogate dam may, optionally, further comprise fertilizing one or more oocytes or ovum by in vitro fertilization and implanting the fertilized embryo(s) into one or more members of the population of recipient bovine surrogate dams and then identifying an association of the microbiome profiles with a high success rate of in vitro fertilization or a low success rate of in vitro fertilization.

In certain aspects of these embodiments that are disclosed herein, vaginal or uterine microbiome profiles of a recipient bovine surrogate dam are indicative of high success of embryo implantation following transfer via in vitro fertilization and suggest a high likelihood that a successful pregnancy will be initiated and/or maintained within the recipient bovine surrogate dam. In other aspects, vaginal or uterine microbiome profiles of a recipient bovine surrogate dam are indicative of low success of embryo implantation following transfer via in vitro fertilization and suggest a low likelihood that a successful pregnancy will be initiated and/or maintained within the recipient bovine surrogate dam. In further aspects, the microbiome profile for one or more of the vaginal or uterine samples that is identified for recipient bovine surrogate dams may be stratified into one or more age ranges.

Within other embodiments, the present disclosure provides methods for improving the success rate of embryo implantation following in vitro fertilization. In certain aspects of these embodiments, the methods comprise obtaining vaginal or uterine fluids comprising a microbiome sample of a candidate recipient bovine surrogate dam. In other aspects, a microbiome profile for one or more of the vaginal or uterine fluids from the recipient bovine surrogate dam indicates that administering an intervention to the recipient bovine surrogate dam would enhance the success rate of embryo implantation and/or maintenance of pregnancy. Such interventions are effective in establishing a recipient bovine surrogate dam a microbiome profile wherein a microbiome composition is associated with a high success rate of in vitro fertilization, embryo implantation, and maintenance of pregnancy.

In certain aspects of the various embodiments disclosed herein, the rate of successful in vitro fertilization is measured by the incidence of embryo implantation. In other aspects, the rate of successful in vitro fertilization is measured by the initiation and/or maintenance of pregnancy. For example, in some aspects, the incidence of embryo implantation and/or initiation of pregnancy is greater than 50%. In other aspects, the incidence of embryo implantation and/or initiation of pregnancy is greater than 60%. In other aspects, the incidence of embryo implantation and/or initiation of pregnancy is greater than 70%. In other aspects, the incidence of embryo implantation and/or initiation of pregnancy is greater than 80%. In other aspects, the incidence of embryo implantation and/or initiation of pregnancy is greater than 90%. In other aspects, the incidence of embryo implantation and/or initiation of pregnancy is greater than 95%.

Interventions or treatments that may be used advantageously to increase the success rate of embryo transfer include one or more of a microbiome supplementation either vaginally or orally. In certain aspects, the intervention may be administered directly to one or more of the vagina or uterus of the recipient bovine surrogate dam. In other aspects, the intervention that is administered to the recipient bovine surrogate dam is effective to provide a microbiome profile of the vaginal and/or uterine fluid wherein the microbiome composition is associated with high success of pregnancy after embryo transfer. In certain aspects the intervention or treatment is administered as a gel. In other aspects the intervention or treatment is administered as a paste. In certain embodiments the intervention or treatment is administered vaginally. In certain embodiments the intervention or treatment is administered orally.

In related embodiments, the present disclosure provides diagnostic test kits for identifying recipient bovine surrogate dams for embryo transfer. In aspects of these embodiments, the diagnostic test kit comprises one or more analytical tools for determining a microbiome profile of one or more of a vaginal or uterine fluid from a recipient bovine surrogate dam. In other aspects, the one or more analytical tools for determining a microbiome profile comprise an apparatus and/or reagents for identifying and measuring bacterial species levels. In further aspects, the one or more analytical tools comprise a portable real-time device for identifying and quantifying bacterial species, and the reagents for use.

Diagnostic test kits according to these embodiments may further comprise a transmitter to communicate/connect a database of one or more microbiome profiles to a device for comparing the microbiome profile of the recipient bovine surrogate dam with the database of one or more microbiome profiles to identify which recipient bovine surrogate dams have microbiome profiles indicative of a high success rate of pregnancy following embryo transfer and/or which recipient bovine surrogate dams have microbiome profiles indicative of a low success rate of pregnancy following embryo transfer.

Diagnostic test kits according to these embodiments may further comprise a database with a collection of microbiome information that is organized so that it can be easily accessed, updated, and compared to other microbiomes, such as a computer database. In some embodiments, the microbiome information in the database is obtained by collecting vaginal samples from a population of recipient bovine surrogate dams, wherein the samples are obtained from one or more of the vaginal or uterine fluid. In aspects of these embodiments, the microbiome information is associated with the ratios and levels of bacteria in the samples and is organized into a microbiome profile. In other aspects, the transmitter to communicate/connect with a database is wired or wireless. In further aspects, the diagnostic test kit may further comprise instructions for use.

According to some embodiments, the present disclosure provides methods for selecting a recipient bovine surrogate dam for embryo transfer, which methods comprise (1) obtaining vaginal fluids from the recipient bovine surrogate dam; (2) identifying a microbiome profile from the vaginal fluids of the recipient bovine surrogate dam; and (3) obtaining one or more embryos for embryo transfer into bovine surrogate dams in which the microbiome profile indicates that the recipient bovine surrogate dam exhibits a high likelihood of a successful embryo implantation and/or maintaining a pregnancy.

According to other embodiments, the present disclosure provides methods for identifying a candidate recipient bovine surrogate dam that is unsuitable for embryo transfer, which methods comprise (1) obtaining vaginal fluids containing a vaginal microbiome from the candidate recipient bovine surrogate dam; (2) identifying a microbiome profile from the vaginal fluids of the candidate recipient bovine surrogate dam; and (3) preventing the candidate recipient bovine surrogate dam from obtaining one or more embryos for embryo transfer, wherein the candidate recipient bovine surrogate dam exhibits a microbiome profile which indicates that the candidate recipient bovine surrogate dam exhibits a low likelihood of a successful embryo implantation and/or maintaining a pregnancy.

In some embodiments, the vaginal or uterine microbiome profile that is indicative of successful embryo implantation and maintenance of pregnancy in the recipient bovine surrogate dam comprises one or more of the following bacterial species: Aerococcaceae_unclassified, planomicrobium, Staphylococcaceae_unclassified, Clostridia_unclassified, Bacteroidales_unclassified, Micrococcaceae_unclassified, paludibacter, brevundimonas, Dietzia, Staphylococcus, Burkholderiales_unclassified, Saccharofermentans, solibacillus, Sporobacter, cystobacteraceae_unclassified, Porphyromonadaceae_unclassified, Longibaculum, Micomonosporaceae_unclassified, Weeksellaceae_unclassified, Geobacillus, Mogibacterium, Actinobacteria_unclassified, Trueperella, Colidextribacter, microvirga, pseudactionotalea, Sphingosinicellaceae_unclassified, Firmicutes_unclassified, Comamonadaceae_unclassified, Paradevosia, Peptoniphilus, Candidatus_Solibacter_unclassified, Neochlamydia, Ruminococcaceae_unclassified, Flaviflexus, Gp3_unclassified, Ramlibacter, Bacteroides, Acetobacteraceae_unclassified, Micrococcales_unclassified, Cytophagales_unclassified, Victivallis, Ohtaekwangia, Parcubacteria_unclassified, Ruminococcus, Erysipelotrichales_unclassified, Planctomycetacia_unclassified, Bifidobacteriaceae_unclassified, Alistipes, Eggerthellaceae_unclassified, Truepera, Roseimaritima, Nocardiaceae_unclassified, Myroides, Flintibacter, Devosia, Limosilactobacillus, Ornithinibacillus, Porphyromonas, Anaerolineaceae_unclassified, Streptococcaceae_unclassified, Faecalibacterium, Bacteroidaceae_unclassified, Verrucomicrobia_unclassified, Caldilineaceae_unclassified, Cellvibrio, Ornithinimicrobium, Gemmatimonadaceae_unclassified, Erysipelotrichaceae_unclassified, Ureaplasma, Xanthomonadaceae_unclassified, Jeotgalicoccus, Verrucomicrobiales_unclassified, Methylobacterium, Monoglobus, Spirochaetaceae_unclassified, Ruminobacter, Paenibacillaceae_1_unclassified, Rhizobiales_unclassified, Neisseriaceae_unclassified, Tumebacillus, Flavobacteriales_unclassified, Bacillaceae 2_unclassified, Petroclostridium, Eubacteriaceae_unclassified, Tissierellaceae_unclassified, Atopobiaceae_unclassified, Mediterranea, Phyllobacterium, Coriobacteriales_unclassified, Raoultibacter, Sphaerobacteraceae_unclassified, Roseburia, Butyricicoccus, Ruminobacter, Oscillibacter, Pseudonocardiaceae_unclassified, Nocardiopsaceae_unclassified, Flavonifractor, Anaerolineae_unclassified, Bacillales_unclassified, Spirochaetaceae_unclassified, Treponema, Paraeggerthella, Monoglobus, Ureaplasma, Succinivibrio, Cellulosilyticum, Fusobacterium, Bacilli_unclassified, Paramuribaculum, Streptococcaceae_unclassified, Subdivision5_unclassified, Planococcaceae_unclassified, Campylobacter, Thermoactinomyces, Micrococcales_unclassified, Alistipes, Aminipila, Paenibacillaceae_1_unclassified, Facklamia, Saccharomonospora, Vaginisenegalia, Nocardiaceae_unclassified, Hespellia, Anaerotignum, Streptococcus, Ruminococcus, Paludibacter, Odoribacter, Devosiaceae_unclassified, Muribaculum, Bacteroidaceae_unclassified, Thermoactinomycetaceae_1_unclassified, Atopostipes, Micrococcaceae_unclassified, Gammaproteobacteria_unclassified, Holdemania, Victivallis, Vulcaniibacterium, Nocardioidaceae_unclassified, Stenotrophomonas, Aquihabitans, Trueperella, Weissella, Flavobacteriales_unclassified, Helcococcus, Mucinivorans, Opitutae_unclassified, Harryflintia, Moraxella, Parvimonas, Sutterella, Kroppenstedtia, Pseudescherichia, Lachnobacterium, Jeotgalicoccus, Kandleria, Longicatena, Succiniclasticum, Terrisporobacter, Ornithinimicrobium, Parabacteroides, Stackebrandtia, Peptoniphilus, Bacillaceae_1_unclassified, Anaerobacillus, Alkalihalobacillus, Caldibacillus, Sphingobacteriaceae_unclassified, Crocinitomix, Membranicola, Cryomorpha, Tumebacillus, Effusibacillus, Alicyclobacillaceae_unclassified, Fibrobacter, Fibrobacteres_unclassified, Aeribacillus, Sphingobacterium, Falsibacillus, Chitinispirillum, and Saprospiraceae_unclassified; in levels that are indicative of successful embryo implantation and maintenance of pregnancy in the recipient bovine surrogate dam.

In some embodiments, the vaginal or uterine microbiome that is indicative of successful embryo implantation and maintenance of pregnancy in the recipient bovine comprises Anaerobutyricum, Azohydromonas, Prevotella, Parvimonas, Frisingincoccus, Gammaproteobacteria Unclassified, Alcaligenaceae Unclassified, Fusobacterium, Sphingomonadaceae Unclassified, Bifidobacteriaceae Unclassified, Bifidobacterium, Erysipelotrichaceae Unclassified, Pelistega, Fusobacteriaceae Unclassified, and Lachnospiraceae Unclassified.

In some embodiments, the vaginal or uterine microbiome that is indicative of successful embryo implantation and maintenance of pregnancy in the recipient beef bovine comprises one or more of the following bacterial species: Aerococcaceae_unclassified, planomicrobium, Staphylococcaceae_unclassified, Clostridia_unclassified, Bacteroidales_unclassified, Micrococcaceae_unclassified, paludibacter, brevundimonas, Dietzia, Staphylococcus, Burkholderiales_unclassified, Saccharofermentans, solibacillus, Sporobacter, cystobacteraceae_unclassified, Porphyromonadaceae_unclassified, Longibaculum, Micomonosporaceae_unclassified, Weeksellaceae_unclassified, Geobacillus, Mogibacterium, Actinobacteria_unclassified, Trueperella, Colidextribacter, microvirga, pseudactionotalea, Sphingosinicellaceae_unclassified, Firmicutes_unclassified, Comamonadaceae_unclassified, Paradevosia, Peptoniphilus, Candidatus_Solibacter_unclassified, Neochlamydia, Ruminococcaceae_unclassified, Flaviflexus, Gp3_unclassified, Ramlibacter, Bacteroides, Acetobacteraceae_unclassified, Micrococcales_unclassified, Cytophagales_unclassified, Victivallis, Ohtaekwangia, Parcubacteria_unclassified, Ruminococcus, Erysipelotrichales_unclassified, Planctomycetacia_unclassified, Bifidobacteriaceae_unclassified, Alistipes, Eggerthellaceae_unclassified, Truepera, Roseimaritima, Nocardiaceae_unclassified, Myroides, Flintibacter, Devosia, Limosilactobacillus, Ornithinibacillus, Porphyromonas, Anaerolineaceae_unclassified, Streptococcaceae_unclassified, Faecalibacterium, Bacteroidaceae_unclassified, Verrucomicrobia_unclassified, Caldilineaceae_unclassified, Cellvibrio, Ornithinimicrobium, Gemmatimonadaceae_unclassified, Erysipelotrichaceae_unclassified, Ureaplasma, Xanthomonadaceae_unclassified, Jeotgalicoccus, Verrucomicrobiales_unclassified, Methylobacterium, Monoglobus, Spirochaetaceae_unclassified, Ruminobacter, Paenibacillaceae_1_unclassified, Rhizobiales_unclassified, Neisseriaceae_unclassified, Tumebacillus, Flavobacteriales_unclassified, Bacillaceae_2_unclassified, and Petroclostridium.

In some embodiments, the vaginal or uterine microbiome that is indicative of successful embryo implantation and maintenance of pregnancy in the recipient dairy bovine comprises one or more of the following bacterial species: Atopobiaceae_unclassified, Eubacteriaceae_unclassified, Tissierellaceae_unclassified, Mediterranea, Phyllobacterium, Coriobacteriales_unclassified, Raoultibacter, Sphaerobacteraceae_unclassified, Roseburia, Butyricicoccus, Ruminobacter, Oscillibacter, Pseudonocardiaceae_unclassified, Nocardiopsaceae_unclassified, Flavonifractor, Anaerolineae_unclassified, Bacillales_unclassified, Spirochaetaceae_unclassified, Treponema, Paraeggerthella, Monoglobus, Ureaplasma, Succinivibrio, Cellulosilyticum, Fusobacterium, Bacilli_unclassified, Paramuribaculum, Streptococcaceae_unclassified, Subdivision5_unclassified, Planococcaceae_unclassified, Campylobacter, Thermoactinomyces, Micrococcales_unclassified, Alistipes, Aminipila, Paenibacillaceae_1_unclassified, Facklamia, Saccharomonospora, Vaginisenegalia, Nocardiaceae_unclassified, Hespellia, Anaerotignum, Streptococcus, Ruminococcus, Paludibacter, Odoribacter, Devosiaceae_unclassified, Muribaculum, Bacteroidaceae_unclassified, Thermoactinomycetaceae_1_unclassified, Atopostipes, Micrococcaceae_unclassified, Gammaproteobacteria_unclassified, Holdemania, Victivallis, Vulcaniibacterium, Nocardioidaceae_unclassified, Stenotrophomonas, Aquihabitans, Trueperella, Flavobacteriales_unclassified, Helcococcus, Mucinivorans, Weissella, Opitutae_unclassified, Harryflintia, Moraxella, Parvimonas, Sutterella, Kroppenstedtia, Pseudescherichia, Lachnobacterium, Jeotgalicoccus, Kandleria, Longicatena, Succiniclasticum, Terrisporobacter, Ornithinimicrobium, Parabacteroides, Stackebrandtia, Peptoniphilus, Bacillaceae_1_unclassified, Anaerobacillus, Alkalihalobacillus, Caldibacillus, Sphingobacteriaceae_unclassified, Crocinitomix, Membranicola, Cryomorpha, Tumebacillus, Effusibacillus, Alicyclobacillaceae_unclassified, Fibrobacter, Fibrobacteres_unclassified, Aeribacillus, Sphingobacterium, Falsibacillus, Chitinispirillum, and Saprospiraceae_unclassified.

Exemplified herein are methods for identifying a candidate bovine surrogate dam that exhibits a vaginal microbiome composition that is predictive of successful embryo implantation following in vitro fertilization, initiation of pregnancy and, in some aspects, maintenance of a successful pregnancy up to and including full term. Also exemplified herein are methods for identifying a candidate bovine surrogate dam that exhibits a vaginal microbiome composition that is predictive of unsuccessful embryo implantation following in vitro fertilization, a low likelihood of initiation of pregnancy and/or a low likelihood of maintenance of a successful pregnancy up to and including full term. In some embodinmets, the vaginal or uterine microbiome that is indicative of successful embryo implantation and maintenance of pregnancy in the recipient bovine comprises diminished levels of microbiota as indicated in the Figures and enumerated herein.

According to some embodiments, the present disclosure provides a method of generating a microbiome database for selecting a recipient bovine surrogate dam for embryo transfer comprising (1) collecting vaginal samples from a population of recipient bovine surrogate dams; (2) identifying a microbiome profile for the vaginal samples from each subject of the population of recipient bovine surrogate dams; and (3) identifying an association of the microbiome profile for the vaginal samples with productive embryo implantation and maintenance of pregnancy.

In other embodiments, the present disclosure provides a method of improving success rate of in vitro fertilization pregnancy comprising (1) obtaining vaginal samples from a recipient bovine surrogate dam; (2) identifying a microbiome profile for the vaginal samples; and (3) administering an intervention to the recipient bovine surrogate dam, wherein the intervention is effective to provide the recipient bovine surrogate dam with a microbiome profile associated with a high level of embryo implantation and maintenance of pregnancy. In some aspects, the intervention that is effective to provide the recipient bovine surrogate dam with a microbiome profile associated with a high level of embryo implantation and maintenance of pregnancy comprises elevating the vaginal and/or uterine levels of Tumebacillus, Flavobacteriales unclassified, Bacillaceae 2 unclassified, Petroclostridium, Mycobacteriales unclassified, Mediterranea, Brevibacterium, Histophilus, Chromatiales unclassified, Micrococcus, Thermus, Kiloniellaceae unclassified, Papillibacter, Jonesia, Solirubrobacter, Rheinheimera, Bacilli unclassified, Muribaculaceae unclassified, Citricoccus, Sporosarcina, Peptoniphilus, Bacillaceae 1 unclassified, Anaerobacillus, Alkalihalobacillus, Caldibacillus, Sphingobacteriaceae unclassified, Crocinitomix, Membranicola, Cryomorpha, Tumebacillus, Effusibacillus, Alicyclobacillaceae unclassified, Fibrobacter, Fibrobacteres_unclassified, Aeribacillus, Sphingobacterium, Falsibacillus, Chitinispirillum, and Saprospiraceae unclassified.

In some embodiments, a microbiome profile that is predictive of unsuccessful embryo implantation and maintenance of pregnancy in a recipient bovine surrogate dam comprises Falcatimonas, Dorea, Pelagibacterium, Lawsonibacterium, Clostridium IV, Planococcaceae unclassified, Sporobacter, Rhodothermales unclassified, Vulcaniibacterium, Rikenellaceae unclassified, Anaerobacter, Alkalibacterium, Harryfintia, Verrucomicrobiales unclassified, Nocardiopsis, Ihubacter, Oscillibacter, Victivallis, Actinomarinicola, Akkermansia, Alistipes, Bacteroidaceae unclassified, Bacteroides unclassified, Bacillales unclassified, Streptosporangiaceae unclassified, Mediterranea, Lactobacillus classified, Turicibacter, Hazenalla, Tissierella, Roseburia, Deinococci unclassified, Gulosibacter, Schaedlerella, Psychrobacillus, Vulgatibacter, Irregularibacter, Cumulibacter, Micrococcales unclassified, Anaerotignum, Cohnella, Clostridia unclassified, Muribaculaceae unclassified, Firmicutes unclassified, Rumunococcaceae unclassified, Arcanobacterium, Flintibacter, Leptotrichia, Succinivibrio, Fusobacteriales inclassified, Treponema, Lactobacillus, Patulibacter, Pseudarthrobacter, Acidbacter unclassified, Sandaracinus, Pseudorhodoferax or Histophilus.

In other embodiments, a microbiome profile that is predictive of unsuccessful embryo implantation and maintenance of pregnancy in a recipient bovine surrogate dam comprises Victivallis, Pseudorhodoferax, Rikenellaceae Unclassified, Clostridia Unclassified, Monoglobus, Akkermansia, Ihubacter, Mediterranea, Alistipes, Bacteria Unclassified, and Bacteroidetes Unclassified.

According other embodiments, the present disclosure provides diagnostic test kits for selecting recipient bovine surrogate dam for embryo transfer comprising: (1) one or more analytical tools for determining a microbiome profile of a vaginal sample from the recipient bovine surrogate dam; (2) a transmitter to communicate/connect a database of one or more microbiome profiles of vaginal samples; (3) a device for comparing the microbiome profile of the vaginal sample from the recipient bovine surrogate dam and with the database of one or more microbiome profiles to identify a recipient bovine surrogate dam has a microbiome profile that is indicative of a high or a low level of embryo implantation and maintenance of pregnancy; and (4) and instructions for use.

### EXAMPLES

The present disclosure is further described with reference to the following examples, which are provided to illustrate certain embodiments and are not intended to limit the scope of the present disclosure or the subject matter claimed.

### Example 1

### Analysis of Microbiome Profiles that are Associated with a High or Low Rate of Embryo Implantation and Maintenance of Pregnancy in Vaginal Fluid of Candidate Cows

This Example demonstrates that certain microbiome ratios in the vaginal fluid of a candidate cow is predictive of a high or low rate of embryo implantation following in vitro fertilization and high or low probability, respectively, that a pregnancy will be maintained to full term. Specific vaginal microbiome profiles were identified that are reflective or predictive of the ability of a surrogate dam to establish a successful pregnancy following embryo transfer.

Results (methods) description: Sequencing of the V3-V4 region of the 16S rRNA gene was conducted.

Relative abundances: Not absolute count: It does not count number of bacteria; but illustrates the proportions in relation to the other bacteria found in each sample. Takes into consideration only the main bacteria (more than 1%). Primers are used to amplify specific bacteria and the bacteria is able to be quantified in relation to other bacteria in the sample yielding the relative abundance of specific bacteria per sample.

Principal Coordinate Analysis (PCoA): Used to illustrate similarities between bacterial communities. Represents the similarity between bacteria in each sample. The closer the dots, the more similar in their microbiota. The analysis of molecular variance (AMOVA) is the statistical test used to find significant differences between the groups. Membership: considers only presence or absence of bacteria (all bacteria are included). The PCoA is developed from constructing a distance matrix from a table that contains the number of times a species is found in a group/community. The use of 2 axes were sufficient to explain 75% of data variation.

LEfSe (LDA score>2): LDA Effect Size (LEfSe) (Segata et. al 2010) is an algorithm for High-Dimensional biomarker discovery and explanation that identifies taxa characterizing the differences between two or more biological conditions. It emphasizes both statistical significance and biological relevance, allowing researchers to identify differentially abundant features that are also consistent with biologically meaningful categories (subclasses). It uses the non-parametric factorial Kruskal-Wallis (KW) sum-rank test to detect features with significant differential abundance with respect to the class of interest; biological significance is subsequently investigated using a set of pairwise tests among subclasses using the (unpaired) Wilcoxon rank-sum test. As a last step, LEfSe uses Linear Discriminant Analysis to estimate the effect size of each differentially abundant features.
490 samples
247 Beef
Several locations in Texas and Kansas
243 Dairy
All samples from a single owned dairy system in Kansas (multiple different locations, heifers and cows)
245 pregnant recipient samples, 245 open recipient samples
Sequencing conducted at FERA
Sequencing of the V3-V4 region of the 16S rRNA gene
The analysis started with 15,901,181 reads and finished with 10,886,618 good quality reads after data cleanup.

Vaginal fluids can be collected and for subsequent laboratory analysis to establish bacterial profiles and ratios that convey the ability or inability of a recipient bovine animal to become pregnant via an embryo transfer and thus are desirable candidate animals for serving as surrogate dams for in vitro fertilization derived embryos.

Specific microbiome profiles have been identified that reflect/predict the ability or inability of a surrogate dam to recognize and maintain an embryo pregnancy. The specific microbiome profiles are able to categorize a surrogate dam as being likely or not likely to become pregnant with approximately an 60% accuracy. Currently the selection method available to identify if a surrogate dam is a candidate for embryo transfer is the presence of a Corpus Luteum at time of transfer. The ability to identify the microbiome profile of a surrogate dam at or prior to embryo transfer can increase the selection accuracy of candidate surrogate dams resulting in a higher overall pregnancy rate.

Significant differences existed between pregnant and non-pregnant recipients regardless of breed type, however individual animal variation was high. 26 microbiota showed significant differential abundance between pregnant and nonpregnant recipients when all breed types were included together in the analysis.

Significant differences existed between pregnant and non-pregnant recipients within breed type, however individual animal variation was high. Beef: 3 microbiota that showed significant differential abundance between pregnant and non-pregnant recipients. Dairy: 43 microbiota that showed significant differential abundance between pregnant and non-pregnant recipients.

Significantly different microbiota populations were found between the beef and dairy recipients.

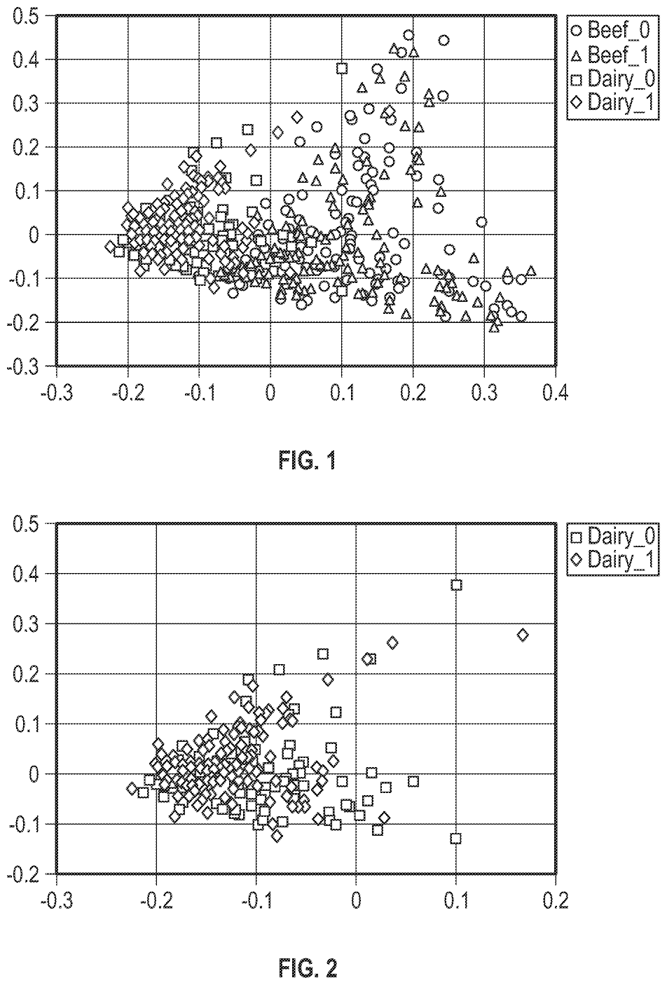

FIG. 1 illustrates Principal Coordinate Analysis (PCoA) of bacteria by breed type and pregnancy status post embryo transfer based on bacteria membership.

PCoA graphically illustrates the similarity between bacteria in each sample. The closer the dots, the more similar in their microbiota.

This PCoA focuses on bacterial membership considering only presence or absence of bacteria (all bacteria are included).

The analysis of molecular variance (AMOVA) is the statistical test used to find significant differences between the groups.

FIG. 2 illustrates Principal Coordinate Analysis (PCoA) of the bacteria membership within dairy recipients based on pregnancy status post embryo transfer.

PCoA graphically illustrates the similarity between bacteria in each sample. The closer the dots, the more similar in their microbiota.

This PCoA focuses on bacterial membership considering only presence or absence of bacteria (all bacteria are included).

The analysis of molecular variance (AMOVA) is the statistical test used to find significant differences between the groups.

AMOVA:

| | | |
|---|---|---|
| Dairy_0-Dairy_1 | Among | Within Total |

| | | | |
|---|---|---|---|
| SS | 0.382604 | 35.7247 | 36.1073 |
| df | 1 | 235 | 236 |
| MS | 0.382604 | 0.15202 | |
| Fs: | 2.5168 | | |
| p-value: | <0.001* | | |

The AMOVA indicates a significant difference in the microbiota membership between pregnant vs non-pregnant dairy recipients.

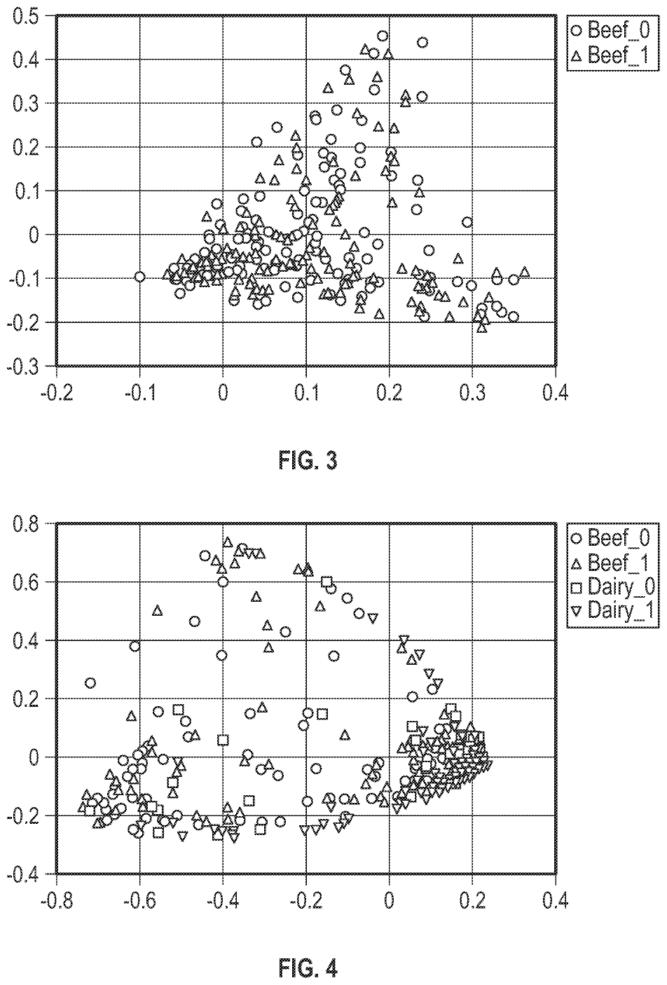

FIG. 3 illustrates Principal Coordinate Analysis (PCoA) of the bacteria membership within beef recipients based on pregnancy status post embryo transfer:

PCoA graphically illustrates the similarity between bacteria in each sample. The closer the dots, the more similar in their microbiota.

This PCoA focuses on bacterial membership considering only presence or absence of bacteria (all bacteria are included).

The analysis of molecular variance (AMOVA) is the statistical test used to find significant differences between the groups.

AMOVA:

| | | |
|---|---|---|
| Beef_0-Beef_1 | Among | Within Total |

| | | | |
|---|---|---|---|
| SS | 0.212433 | 52.4256 | 52.6381 |
| df | 1 | 246 | 247 |
| MS | 0.212433 | 0.213112 | |
| Fs: | 0.99681 | | |
| p-value: | 0.381 | | |

The AMOVA indicates no significant difference in the microbiota membership between pregnant vs non-pregnant beef recipients.

FIG. 4 illustrates Principal Coordinate Analysis (PCoA) of bacteria by breed type and pregnancy status post embryo transfer based on bacteria structure.

PCoA graphically illustrates the similarity between bacteria in each sample. The closer the dots, the more similar in their microbiota.

This PCoA focuses on bacterial structure considering presence or absence of bacteria (all bacteria are included) as well as their relative abundances.

The analysis of molecular variance (AMOVA) is the statistical test used to find significant differences between the groups.

Results from the AMOVA showed no significant differences between the biological groups of pregnant vs non pregnant.

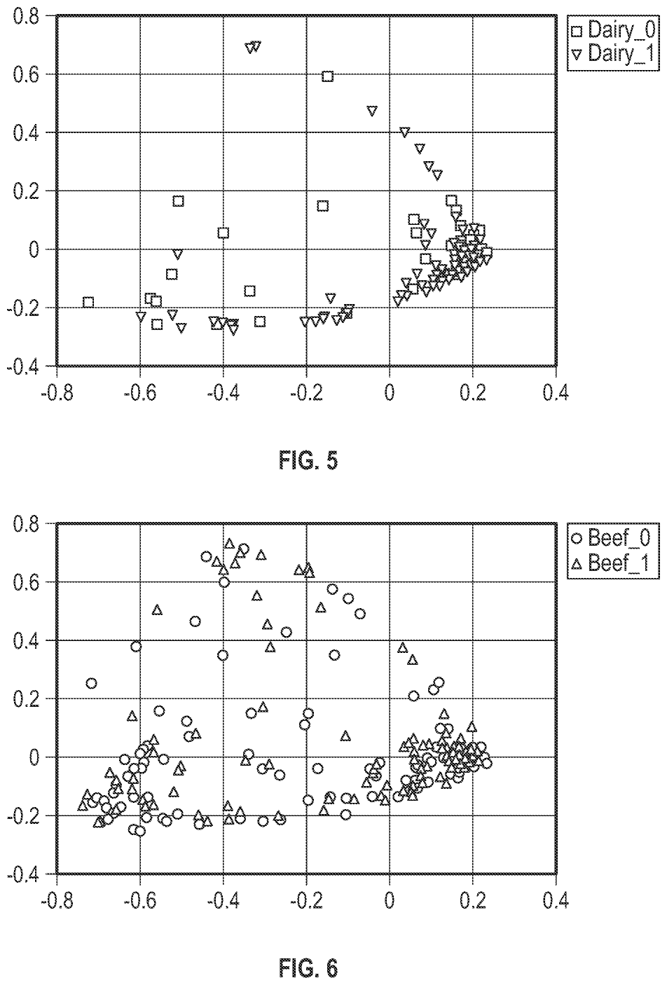

FIG. 5 illustrates Principal Coordinate Analysis (PCoA) of bacteria structure in dairy recipients based on pregnancy status post embryo transfer.

PCoA graphically illustrates the similarity between bacteria in each sample. The closer the dots, the more similar in their microbiota.

This PCoA focuses on bacterial structure considering presence or absence of bacteria (all bacteria are included) as well as their relative abundances.

The analysis of molecular variance (AMOVA) is the statistical test used to find significant differences between the groups.

AMOVA:

| | | |
|---|---|---|
| Dairy_0-Dairy_1 | Among | Within Total |

| | | | |
|---|---|---|---|
| SS | 0.265849 | 22.0404 | 22.3062 |
| df | 1 | 235 | 236 |
| MS | 0.265849 | 0.0937887 | |
| Fs: | 2.83455 | | |
| p-value: | 0.025 | | |

Significant difference in the microbiota structure between pregnant vs non-pregnant dairy recipients.

FIG. 6 illustrates Principal Coordinate Analysis (PCoA) of bacteria structure in beef recipients based on pregnancy status post embryo transfer.

PCoA graphically illustrates the similarity between bacteria in each sample. The closer the dots, the more similar in their microbiota.

This PCoA focuses on bacterial structure considering presence or absence of bacteria (all bacteria are included) as well as their relative abundances.

The analysis of molecular variance (AMOVA) is the statistical test used to find significant differences between the groups.

AMOVA:

| | | |
|---|---|---|
| Beef_0-Beef_1 | Among | Within Total |

| | | | |
|---|---|---|---|
| SS | 0.108306 | 61.6917 | 61.8 |
| df | 1 | 246 | 247 |
| MS | 0.108306 | 0.250779 | |
| Fs: | 0.431877 | | |
| p-value: | 0.855 | | |

A significant difference in the microbiota structure between pregnant vs non-pregnant beef recipients was not observed.

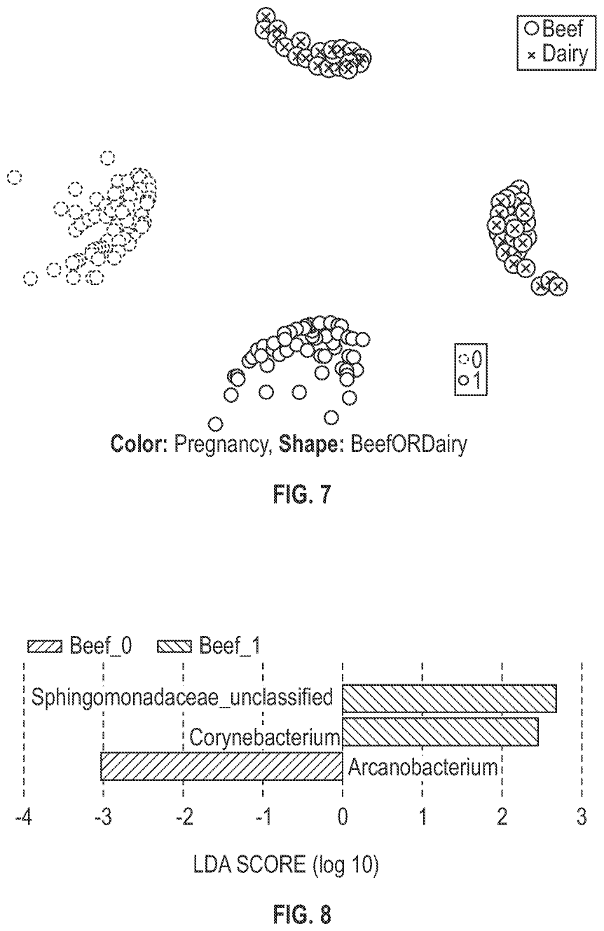

FIG. 7 illustrates a Multidimensional Scaling (MDS) of microbiota structure in beef and dairy recipients based on pregnancy status post embryo transfer using machine learning analysis (Euclidean Distance). It is noted that beef and dairy separate well, and there are very distinct microbiota populations associated with recipients who became pregnant and recipients who did not.

FIG. 8 indicates bacteria with an LDA cut-off of 3 representing discriminative features (bacteria) between beef recipients that became pregnancy versus non-pregnant.

LDA Effect Size (LEfSe) uses an algorpithm to investigate high-dimensional biomarker diversity and identifies taxa characterizing the differences between two biological groups.

The non-parametric factorial Kruskal-Wallis (KW) sum-rank test is used to detect bacteria (features) with significantly differing abundance between biological groups (pregnant vs non-pregnant).

Biological significance is determined using pair-wise tests among the subclasses of the unpaired Wilcoxon rank-sum test.

As a last step pf LEfSe, linear discriminant analysis (LDA) is used to estimate the effect size of each differentially abundant feature (bacteria) between the biological groups of pregnant versus non-pregnant.

Only microbiota that are positively influencing pregnancy would be viable candidates for a vaginal treatment prior to or at transfer.

As the microbiome changes throughout the Estrous cycle using microbiota that are negatively influencing pregnancy would only be a viable test at day of transfer and this is logistically not ideal.

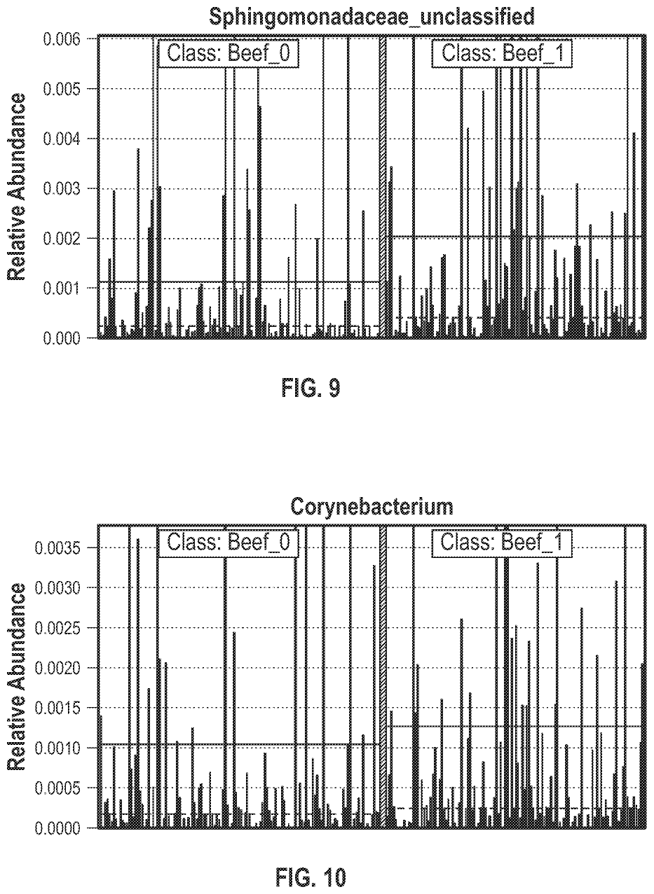

FIG. 9 is graphical representation of the significant difference in relative abundance of the bacteria Sphingomandaceae unclassified on pregnant versus non-pregnant beef recipients after embryo transfer.

The Y axis is the relative abundance, individual animals are represented by the red vertical lines.

The mean abundance by group is indicated by a solid line, the median is represented by the dashed line.

The mean relative abundance of Sphingomandaceae unclassified was significantly higher in beef recipients who became pregnant.

FIG. 10 is graphical representation of the significant difference in relative abundance of Corynebacterium the on pregnant versus non-pregnant beef recipients after embryo transfer.

The Y axis is the relative abundance, individual animals are represented by the red vertical lines.

The mean abundance by group is indicated by a solid line, the median is represented by the dashed line.

The mean relative abundance of Corynebacterium was significantly higher in beef recipients who became pregnant.

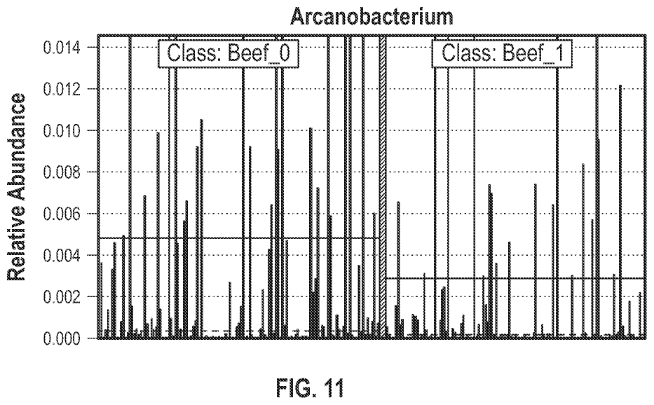

FIG. 11 is graphical representation of the significant difference in relative abundance of Arcanobacterium the in pregnant versus non-pregnant beef recipients after embryo transfer.

The Y axis is the relative abundance, individual animals are represented by the red vertical lines.

The mean abundance by group is indicated by a solid line, the median is represented by the dashed line.

The mean relative abundance of Arcanobacterium was significantly higher in beef recipients who did not become pregnant.

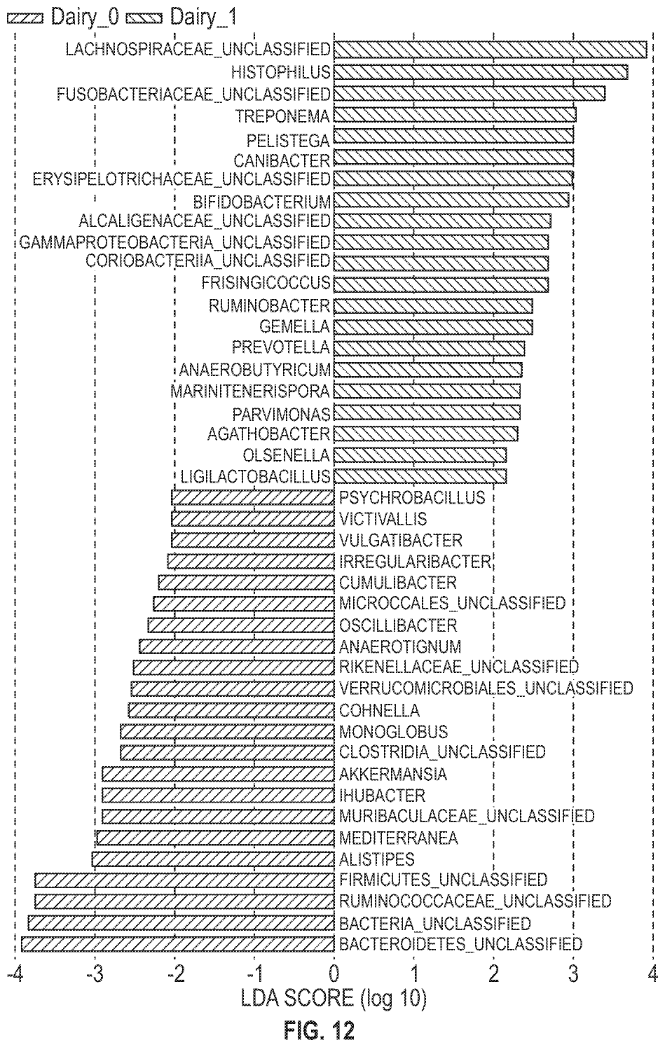

FIG. 12 indicates bacteria with an LDA cut-off of 4 between dairy recipients based on pregnancy status post embryo transfer.

LDA Effect Size (LEfSe) uses an algorithm to investigate high-dimensional biomarker diversity and identifies taxa characterizing the differences between two biological groups.

The non-parametric factorial Kruskal-Wallis (KW) sum-rank test is used to detect bacteria (features) with significantly differing abundance between biological groups (pregnant vs non-pregnant).

Biological significance is determined using pair-wise tests among the subclasses of the unpaired Wilcoxon rank-sum test.

As a last step pf LEfSe, linear discriminant analysis (LDA) is used to estimate the effect size of each differentially abundant feature (bacteria) between the biological groups of pregnant versus non-pregnant.

Only microbiota that are positively influencing pregnancy would be viable candidates for a vaginal treatment prior to or at transfer.

As the microbiome changes throughout the Estrous cycle using microbiota that are negatively influencing pregnancy would only be a viable test at day of transfer and this is logistically not ideal.

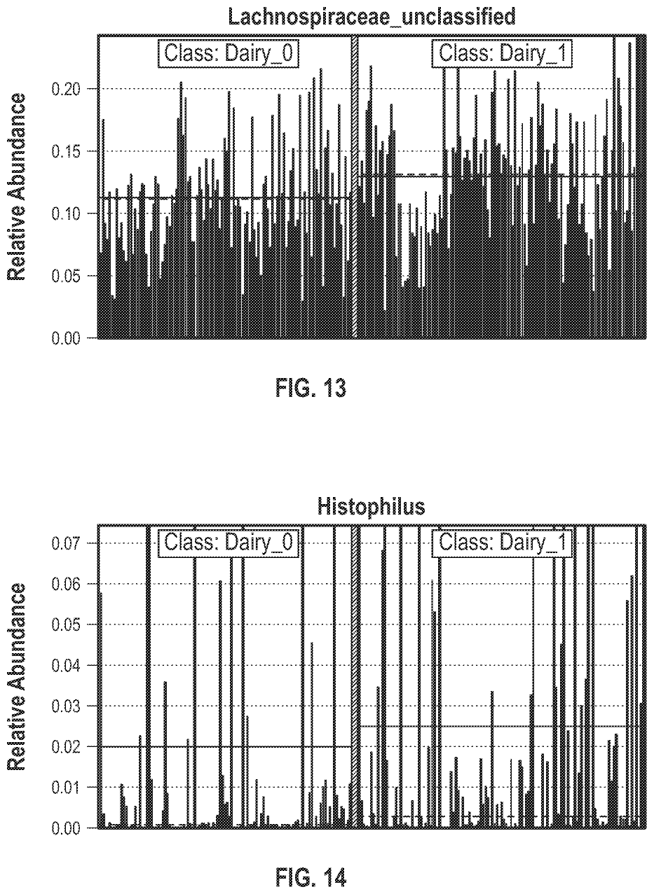

FIG. 13 is graphical representation of the significant difference in relative abundance of Lachnospiraceae unclassified the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Lachnospiraceae unclassified was significantly higher in dairy recipients who became pregnant.

FIG. 14 is graphical representation of the significant difference in relative abundance of Histophilus the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Histophilus was significantly higher in dairy recipients who became pregnant.

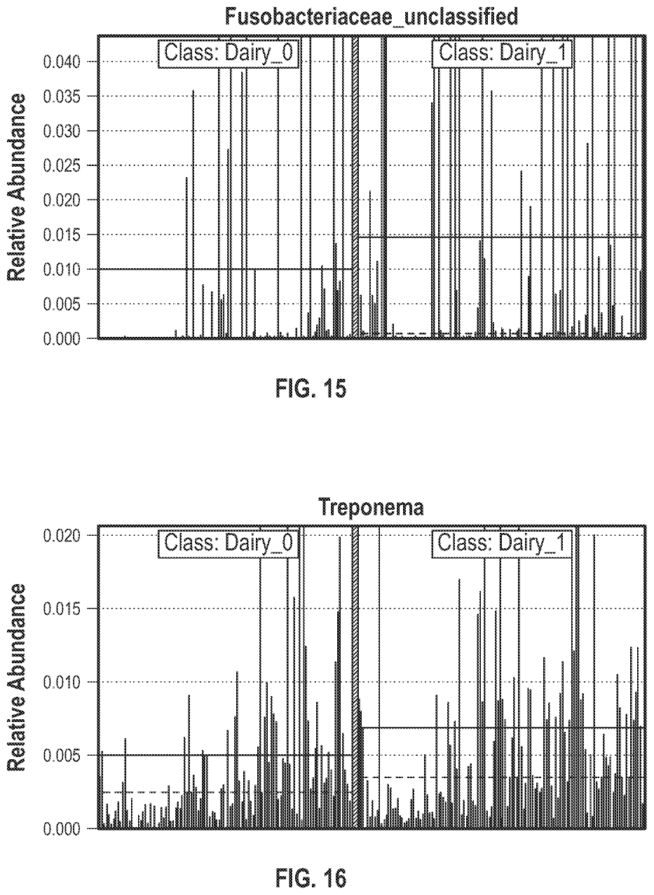

FIG. 15 is graphical representation of the significant difference in relative abundance of Fusobacteriaceae unclassified the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Fusobacteriaceae unclassified was significantly higher in dairy recipients who became pregnant.

FIG. 16 is graphical representation of the significant difference in relative abundance of Treponema the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Treponema was significantly higher in dairy recipients who became pregnant.

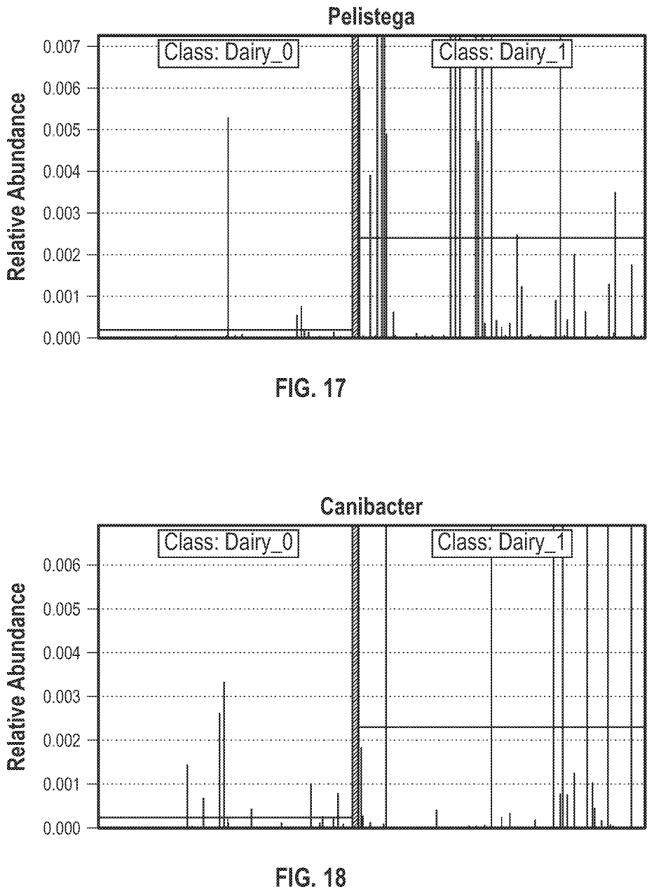

FIG. 17 is graphical representation of the significant difference in relative abundance of Pelistega the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Pelistega was significantly higher in dairy recipients who became pregnant.

FIG. 18 is graphical representation of the significant difference in relative abundance of Canibacter the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Canibacter was significantly higher in dairy recipients who became pregnant.

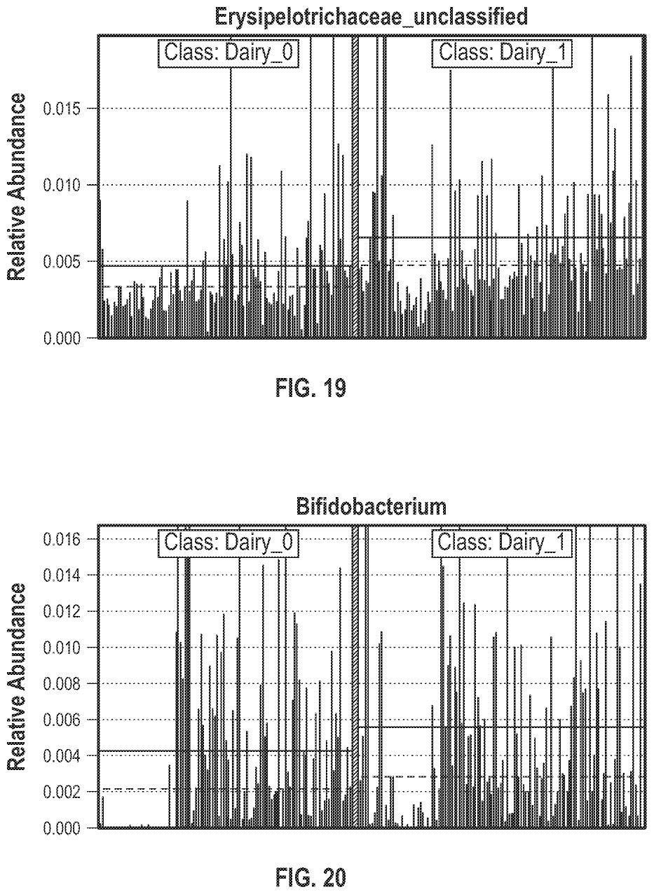

FIG. 19 is graphical representation of the significant difference in relative abundance of Erysipelotricheae unclassified the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Erysipelotricheae unclassified was significantly higher in dairy recipients who became pregnant.

FIG. 20 is graphical representation of the significant difference in relative abundance of Bifidobacterium the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Bifidobacterium was significantly higher in dairy recipients who became pregnant.

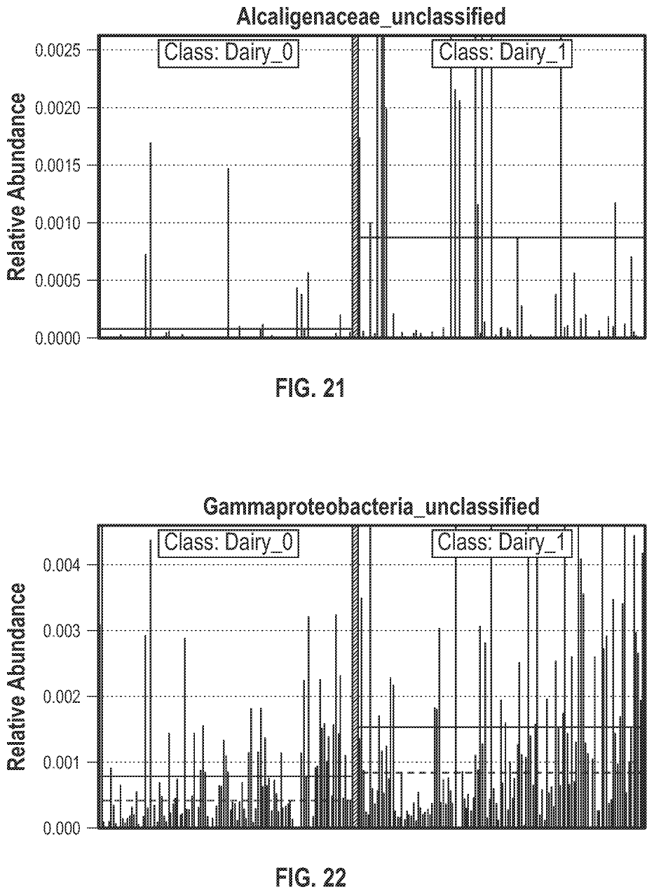

FIG. 21 is graphical representation of the significant difference in relative abundance of Alcaligenaceae unclassified the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Alcaligenaceae unclassified was significantly higher in dairy recipients who became pregnant.

FIG. 22 is graphical representation of the significant difference in relative abundance of Gammaproteabacteria unclassified the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Gammaproteabacteria unclassified was significantly higher in dairy recipients who became pregnant.

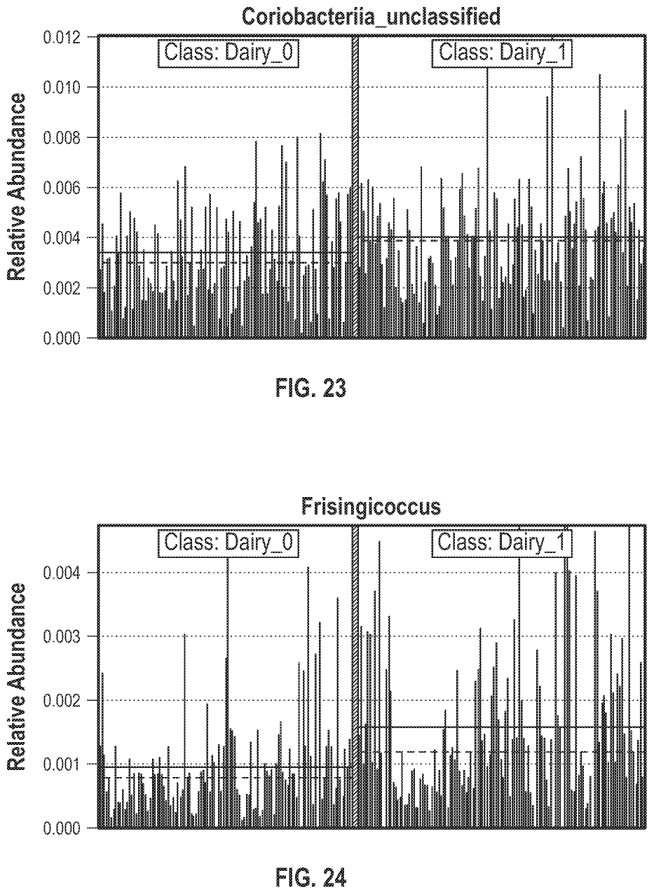

FIG. 23 is graphical representation of the significant difference in relative abundance of Coriobacteria unclassified the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Coriobacteria unclassified was significantly higher in dairy recipients who became pregnant.

FIG. 24 is graphical representation of the significant difference in relative abundance of Frisingicoccus the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Frisingicoccus was significantly higher in dairy recipients who became pregnant.

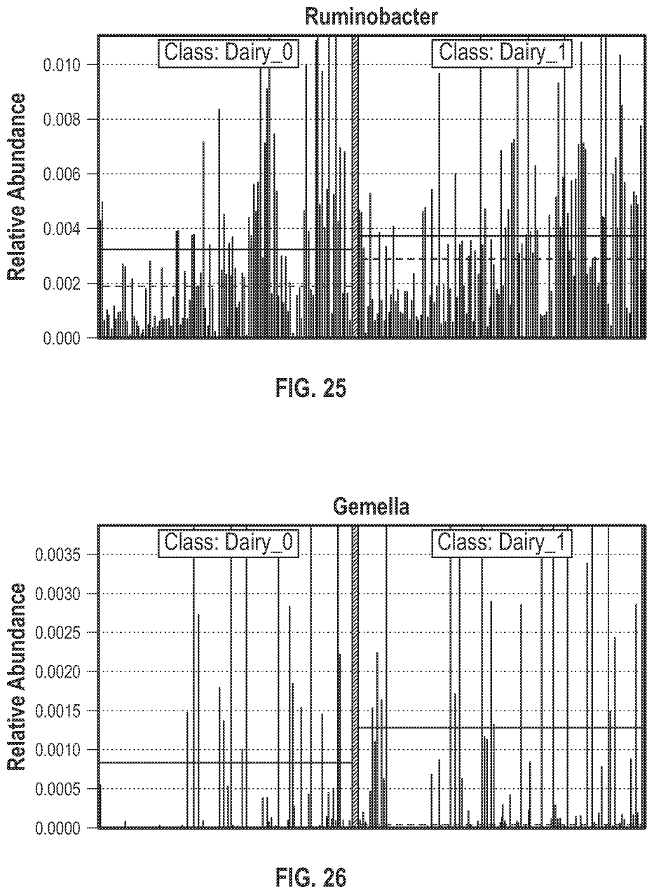

FIG. 25 is graphical representation of the significant difference in relative abundance of Ruminobacter the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Ruminobacter was significantly higher in dairy recipients who became pregnant.

FIG. 26 is graphical representation of the significant difference in relative abundance of Gemella the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Gemella was significantly higher in dairy recipients who became pregnant.

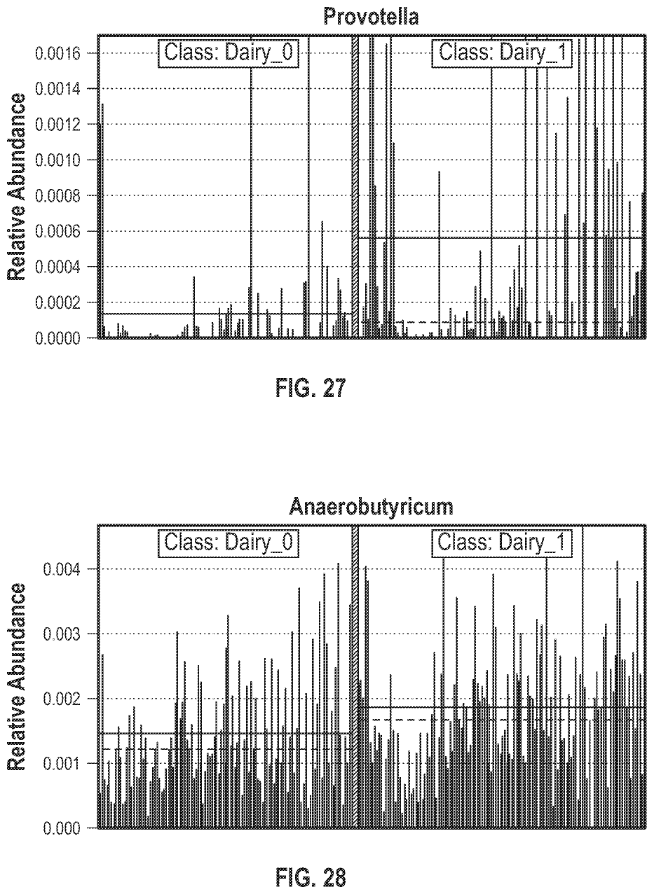

FIG. 27 is graphical representation of the significant difference in relative abundance of Prevotella the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Prevotella was significantly higher in dairy recipients who became pregnant.

FIG. 28 is graphical representation of the significant difference in relative abundance of Anaerobutyricum the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Anaerobutyricum was significantly higher in dairy recipients who became pregnant.

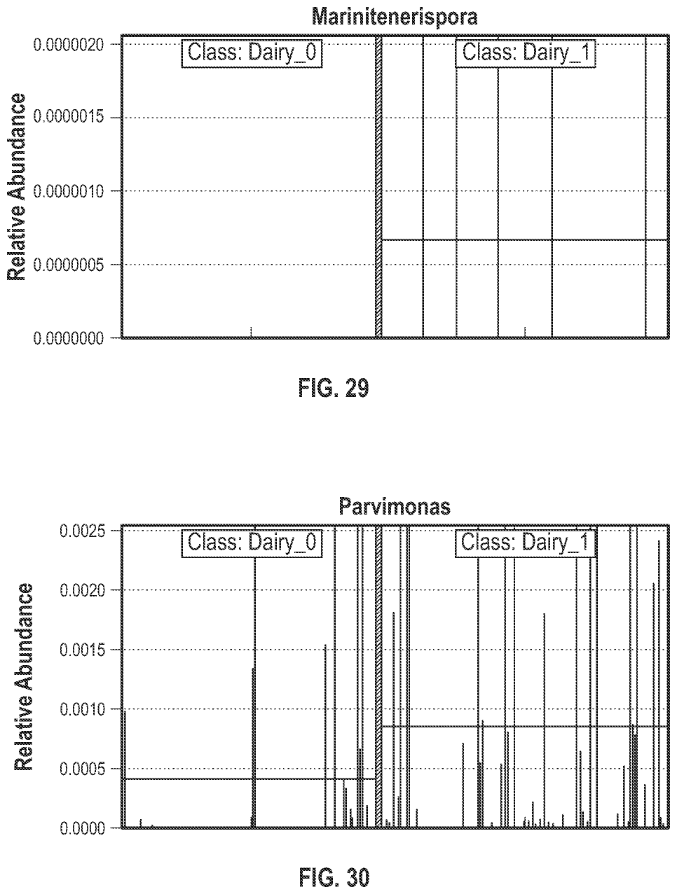

FIG. 29 is graphical representation of the significant difference in relative abundance of Marinitenerispora the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Marinitenerispora was significantly higher in dairy recipients who became pregnant.

FIG. 30 is graphical representation of the significant difference in relative abundance of Parvimonas the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Parvimonas was significantly higher in dairy recipients who became pregnant.

FIG. 31 is graphical representation of the significant difference in relative abundance of Agathobacter the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Agathobacter was significantly higher in dairy recipients who became pregnant.

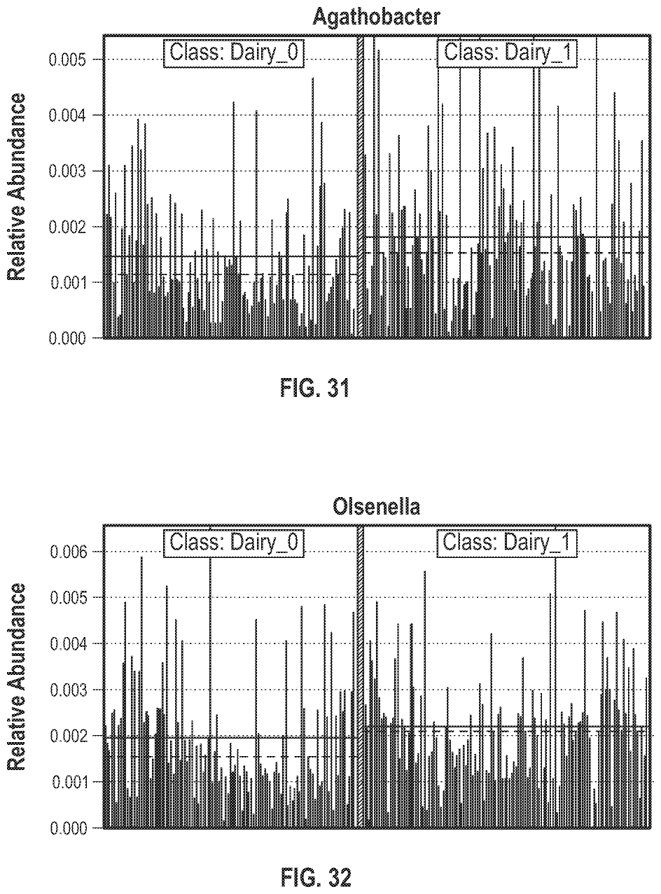

FIG. 32 is graphical representation of the significant difference in relative abundance of Olsenella the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Olsenella was significantly higher in dairy recipients who became pregnant.

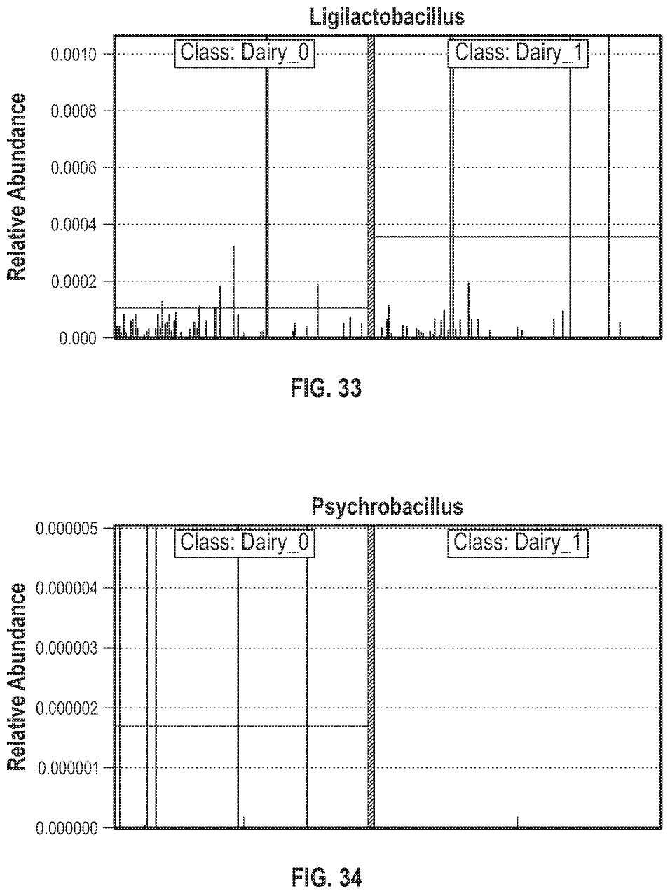

FIG. 33 is graphical representation of the significant difference in relative abundance of Ligilactobacullus the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Ligilactobacullus was significantly higher in dairy recipients who became pregnant.

FIG. 34 is graphical representation of the significant difference in relative abundance of Psychrobacillus the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Psychrobacillus was significantly higher in dairy recipients who did not became pregnant.

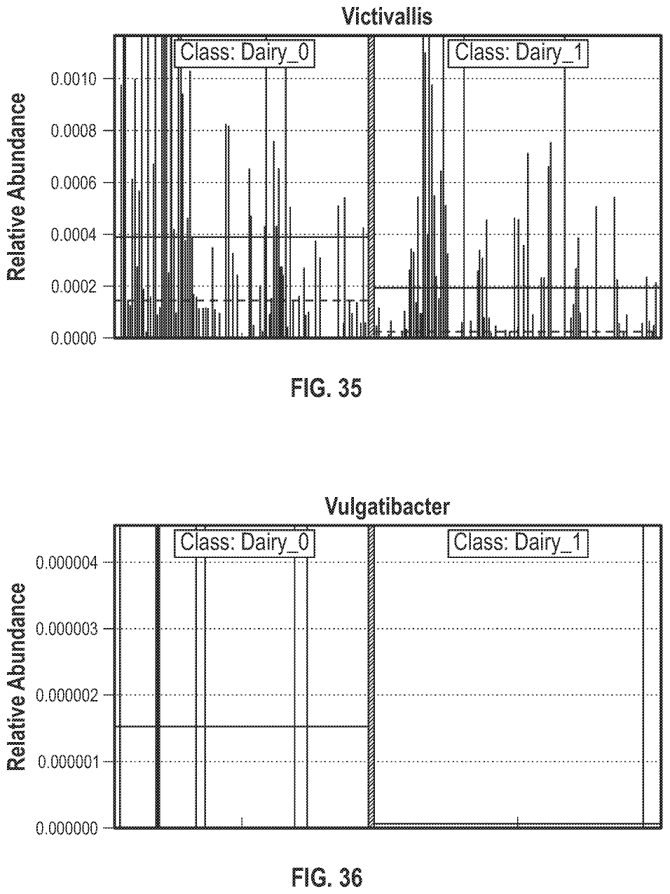

FIG. 35 is graphical representation of the significant difference in relative abundance of Victivallis the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Victivallis was significantly higher in dairy recipients who did not became pregnant.

FIG. 36 is graphical representation of the significant difference in relative abundance of Vulgatibacter the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Vulgatibacter was significantly higher in dairy recipients who did not became pregnant.

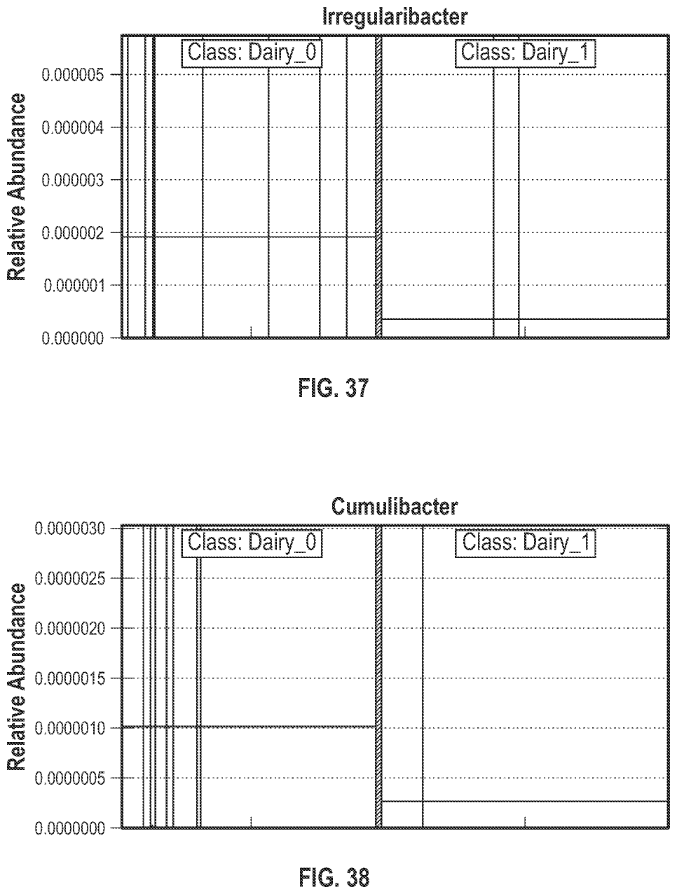

FIG. 37 is graphical representation of the significant difference in relative abundance of Irregularibacter the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Irregularibacter was significantly higher in dairy recipients who did not became pregnant.

FIG. 38 is graphical representation of the significant difference in relative abundance of Cumulibacter the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Cumulibacter was significantly higher in dairy recipients who did not became pregnant.

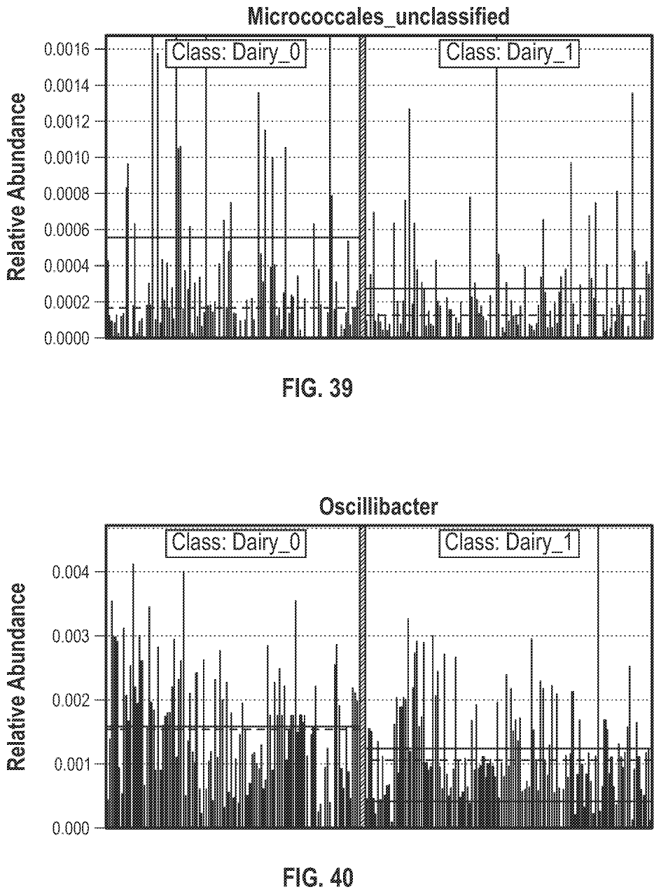

FIG. 39 is graphical representation of the significant difference in relative abundance of Micrococcales unclassified the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Micrococcales unclassified was significantly higher in dairy recipients who did not became pregnant.

FIG. 40 is graphical representation of the significant difference in relative abundance of Oscillibacter the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Oscillibacter was significantly higher in dairy recipients who did not became pregnant.

FIG. 41 is graphical representation of the significant difference in relative abundance of Anaerotignum the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Anaerotignum was significantly higher in dairy recipients who did not became pregnant.

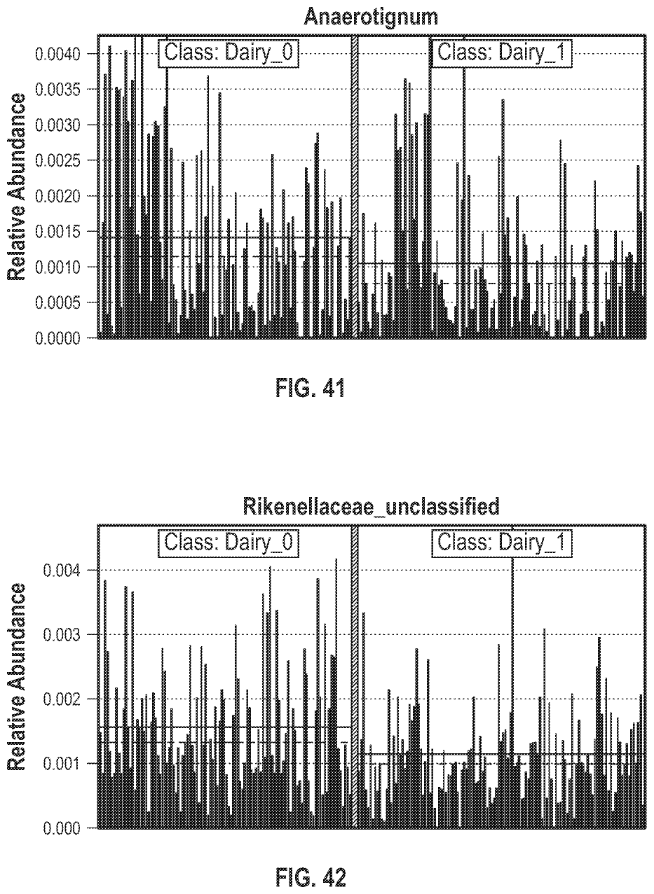

FIG. 42 is graphical representation of the significant difference in relative abundance of Rikenellaceae Unclassified the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Rikenellaceae Unclassified was significantly higher in dairy recipients who did not became pregnant.

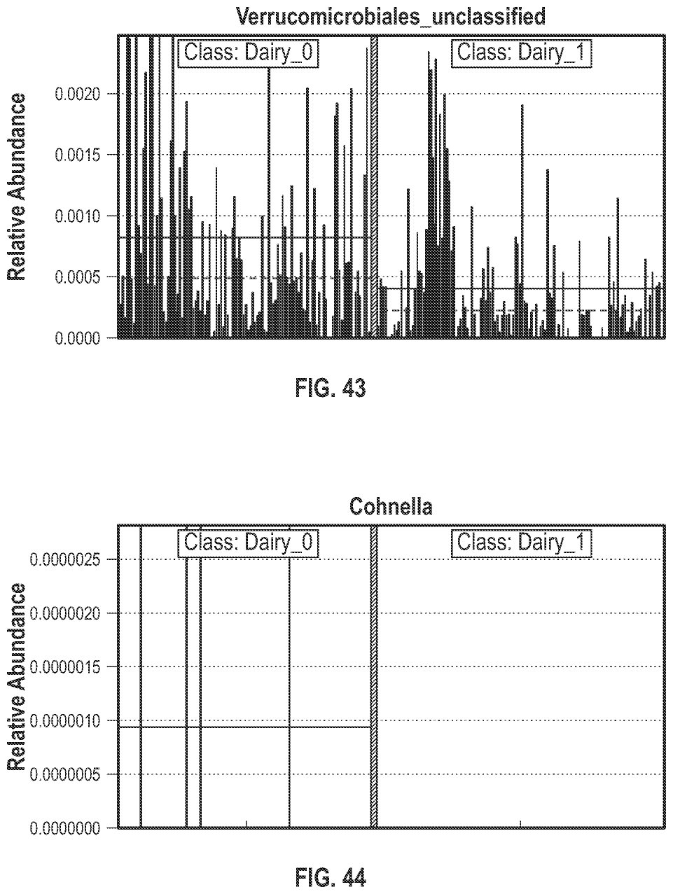

FIG. 43 is graphical representation of the significant difference in relative abundance of Verrucomicrobiales Unclassified the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Verrucomicrobiales Unclassified was significantly higher in dairy recipients who did not became pregnant.

FIG. 44 is graphical representation of the significant difference in relative abundance of Cohnella the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Cohnella was significantly higher in dairy recipients who did not became pregnant.

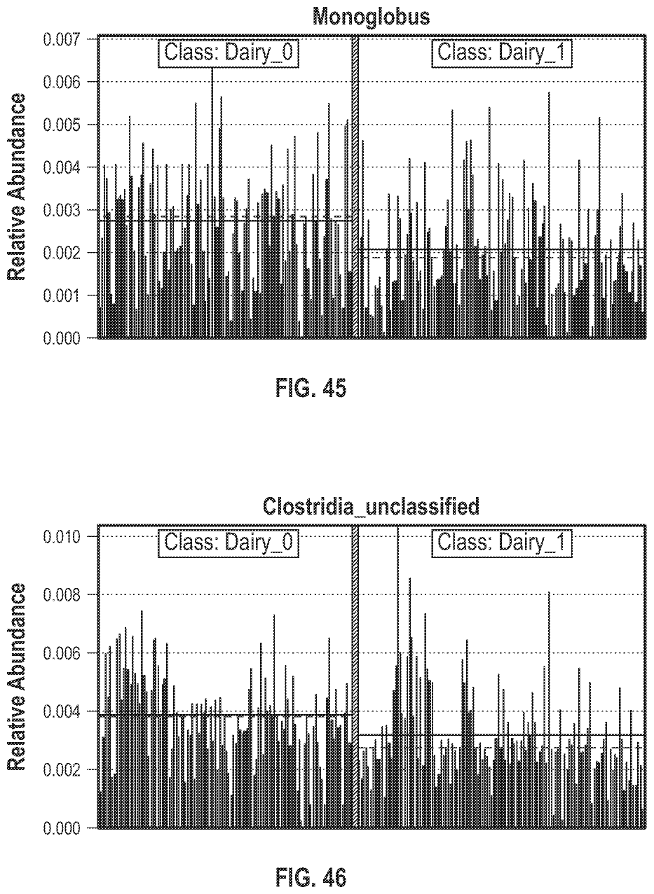

FIG. 45 is graphical representation of the significant difference in relative abundance of Monoglobus the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Monoglobus was significantly higher in dairy recipients who did not became pregnant.

FIG. 46 is graphical representation of the significant difference in relative abundance of Clostridia Unclassified the in pregnant versus non-pregnant dairy recipients after embryo transfer. The Y axis is the relative abundance, individual animals are represented by the red vertical lines.

The mean abundance by group is indicated by a solid line, the median is represented by the dashed line.

The mean relative abundance of Clostridia Unclassified was significantly higher in dairy recipients who did not became pregnant.

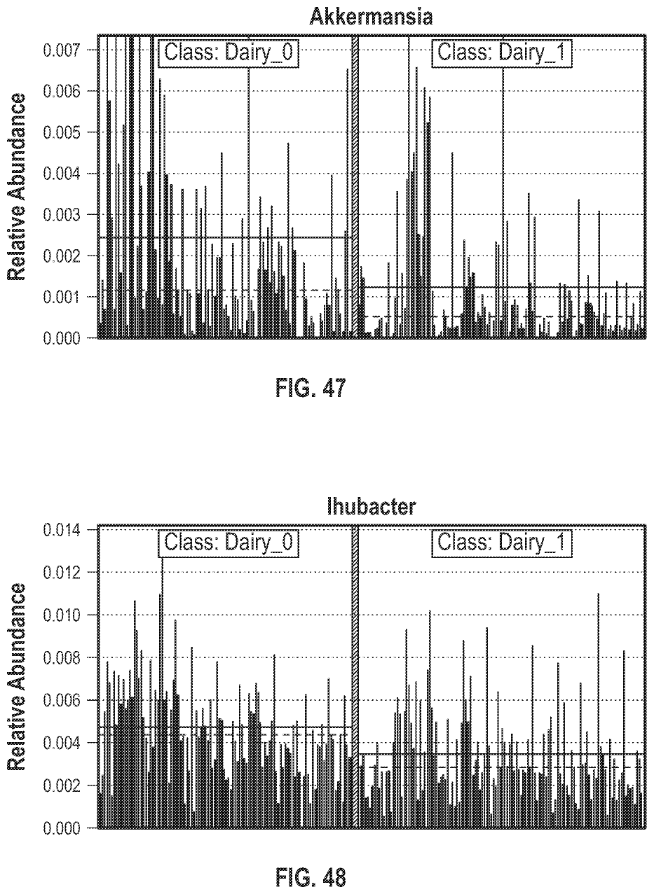

FIG. 47 is graphical representation of the significant difference in relative abundance of Akkermansia the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Akkermansia was significantly higher in dairy recipients who did not became pregnant.

FIG. 48 is graphical representation of the significant difference in relative abundance of Ihubacter the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Ihubacter was significantly higher in dairy recipients who did not became pregnant.

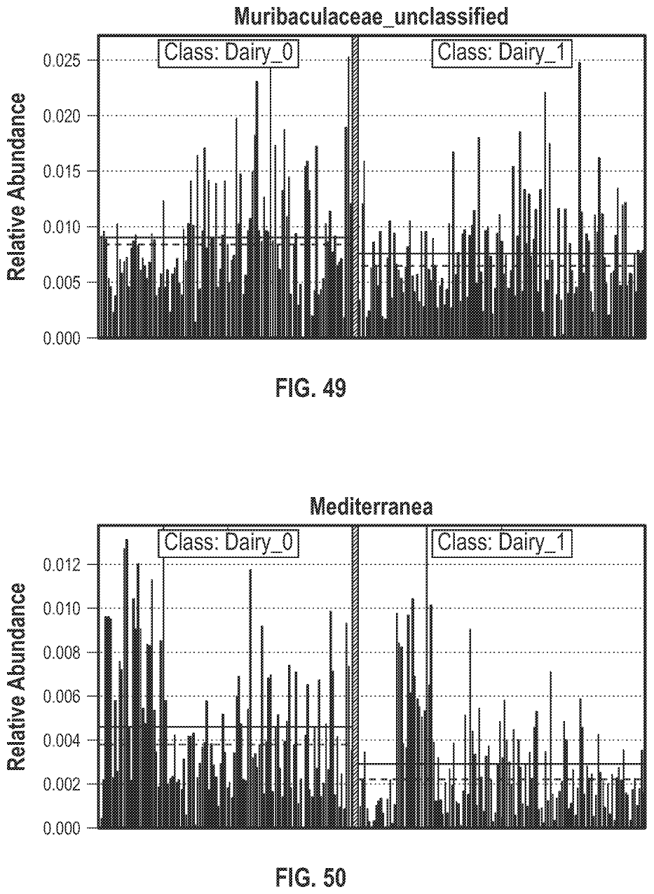

FIG. 49 is graphical representation of the significant difference in relative abundance of Muribaculaceae Unclassified the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Muribaculaceae Unclassified was significantly higher in dairy recipients who did not became pregnant.

FIG. 50 is graphical representation of the significant difference in relative abundance of Mediterranea the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Mediterranea was significantly higher in dairy recipients who did not became pregnant.

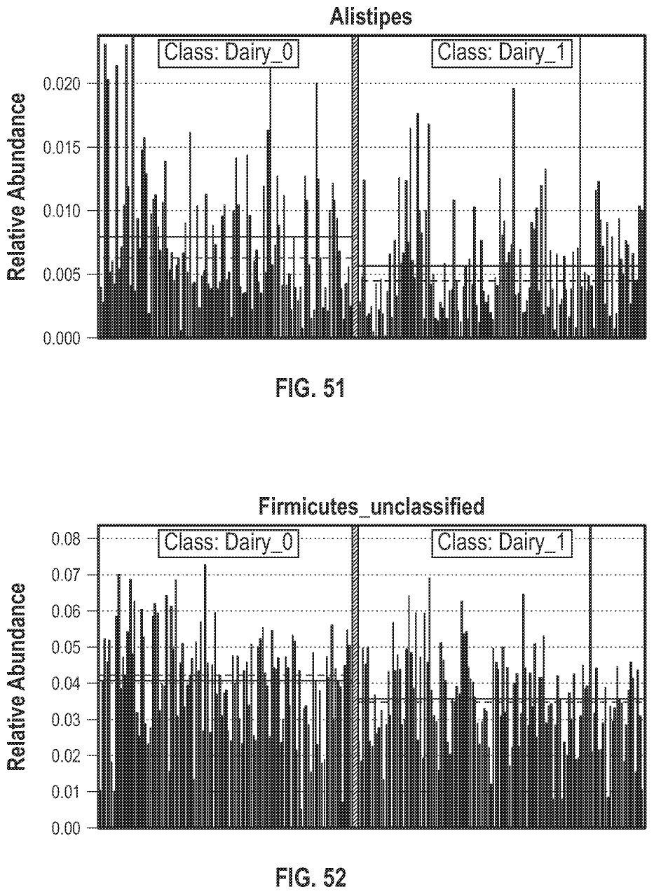

FIG. 51 is graphical representation of the significant difference in relative abundance of Alistipes the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Alistipes was significantly higher in dairy recipients who did not became pregnant.

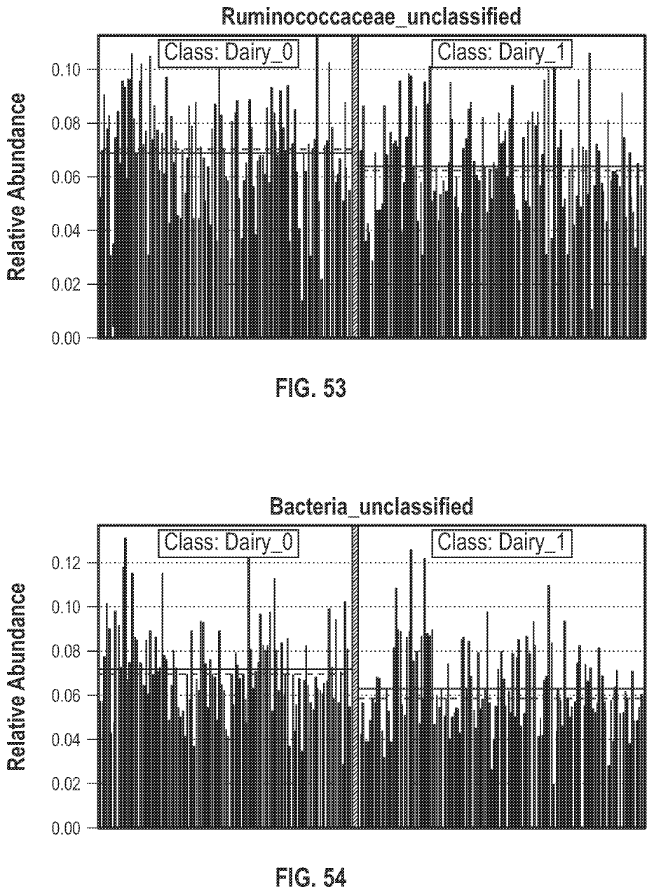

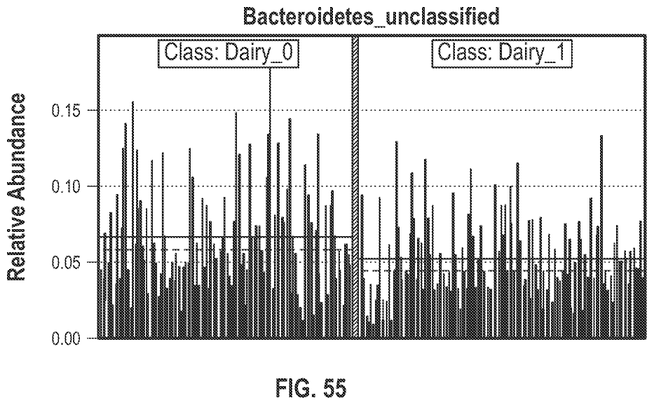

FIG. 52 is graphical representation of the significant difference in relative abundance of Firmicutes Unclassified the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Firmicutes Unclassified was significantly higher in dairy recipients who did not became pregnant.

FIG. 53 is graphical representation of the significant difference in relative abundance of Ruminococcaceae Unclassified the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Ruminococcaceae Unclassified was significantly higher in dairy recipients who did not became pregnant.

FIG. 54 is graphical representation of the significant difference in relative abundance of Bacteria Unclassified the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Bacteria Unclassified was significantly higher in dairy recipients who did not became pregnant.

FIG. 55 is graphical representation of the significant difference in relative abundance of Bacteroidetes Unclassified the in pregnant versus non-pregnant dairy recipients after embryo transfer.

The Y axis is the relative abundance, individual animals are represented by the red vertical lines.

The mean abundance by group is indicated by a solid line, the median is represented by the dashed line.

The mean relative abundance of Bacteroidetes Unclassified was significantly higher in dairy recipients who did not became pregnant.

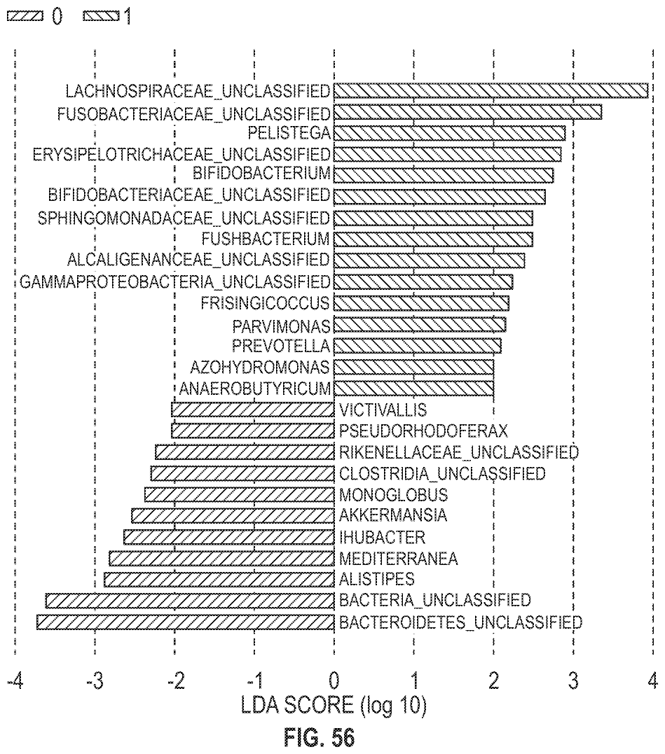

FIG. 56 indicates bacteria with an LDA cut-off of 4 between recipients (regardless of breed type) based on pregnancy status post embryo transfer.

LDA Effect Size (LEfSe) uses an algorpithm to investigate high-dimensional biomarker diversity and identifies taxa characterizing the differences between two biological groups.

The non-parametric factorial Kruskal-Wallis (KW) sum-rank test is used to detect bacteria (features) with significantly differing abundance between biological groups (pregnant vs non-pregnant).

Biological significance is determined using pair-wise tests among the subclasses of the unpaired Wilcoxon rank-sum test.

As a last step pf LEfSe, linear discriminant analysis (LDA) is used to estimate the effect size of each differentially abundant feature (bacteria) between the biological groups of pregnant versus non-pregnant.

Only microbiota that are positively influencing pregnancy would be viable candidates for a vaginal treatment prior to or at transfer.

As the microbiome changes throughout the Estrous cycle using microbiota that are negatively influencing pregnancy would only be a viable test at day of transfer and this is logistically not ideal.

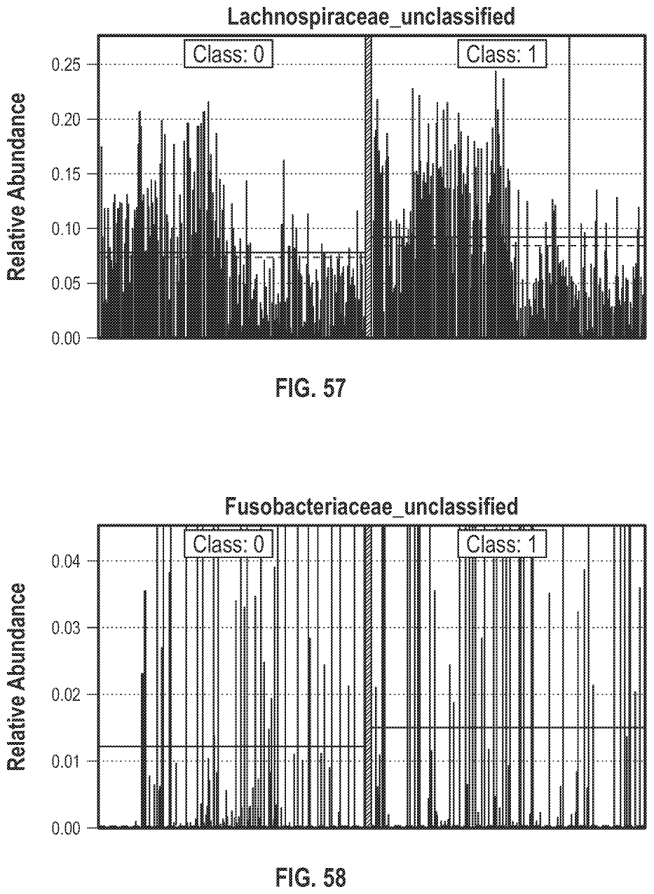

FIG. 57 is graphical representation of the significant difference in relative abundance of Lachnospiraceae Unclassified the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Lachnospiraceae Unclassified was significantly higher in recipients (regardless of breed type) who became pregnant after embryo transfer.

FIG. 58 is graphical representation of the significant difference in relative abundance of Fusobacteriaceae Unclassified the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Fusobacteriaceae Unclassified was significantly higher in recipients (regardless of breed type) who became pregnant after embryo transfer.

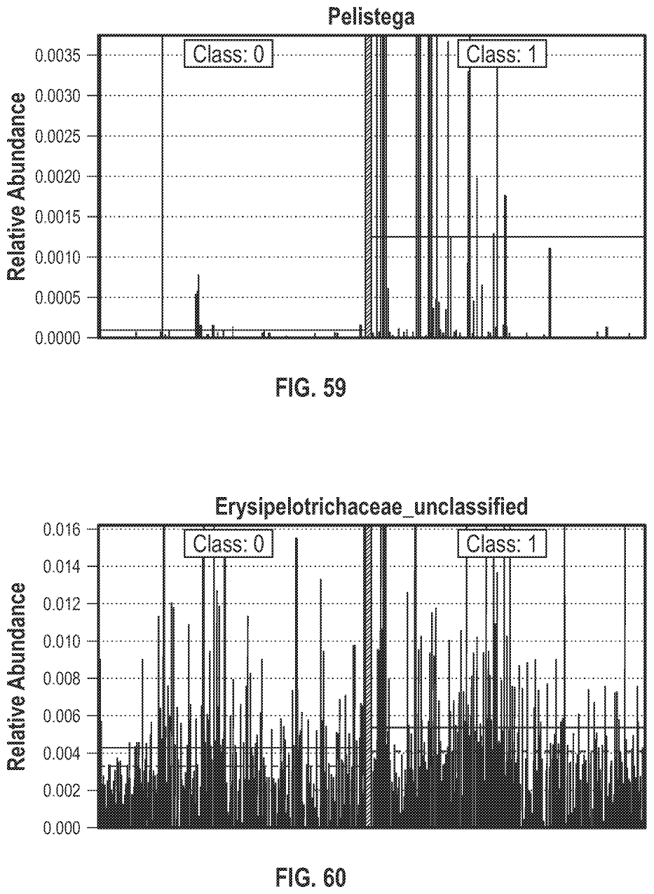

FIG. 59 is graphical representation of the significant difference in relative abundance of Pelistega the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Pelistega was significantly higher in recipients (regardless of breed type) who became pregnant after embryo transfer.

FIG. 60 is graphical representation of the significant difference in relative abundance of Erysipelotrichaceae Unclassified the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Erysipelotrichaceae Unclassified was significantly higher in recipients (regardless of breed type) who became pregnant after embryo transfer.

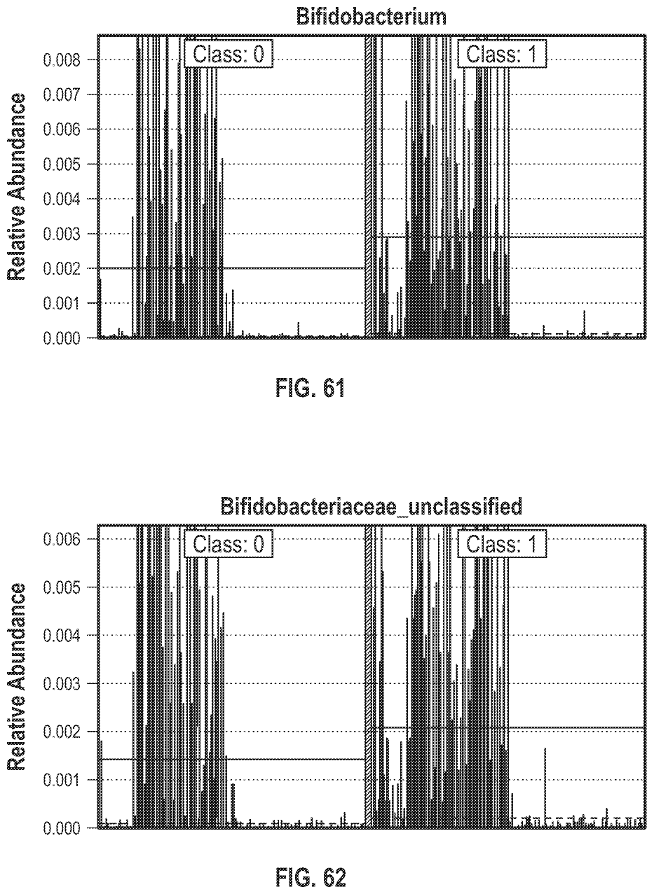

FIG. 61 is graphical representation of the significant difference in relative abundance of Bifidobacterium the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Bifidobacterium was significantly higher in recipients (regardless of breed type) who became pregnant after embryo transfer.

FIG. 62 is graphical representation of the significant difference in relative abundance of Bifidobacteriaceae Unclassified the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Bifidobacteriaceae Unclassified was significantly higher in recipients (regardless of breed type) who became pregnant after embryo transfer.

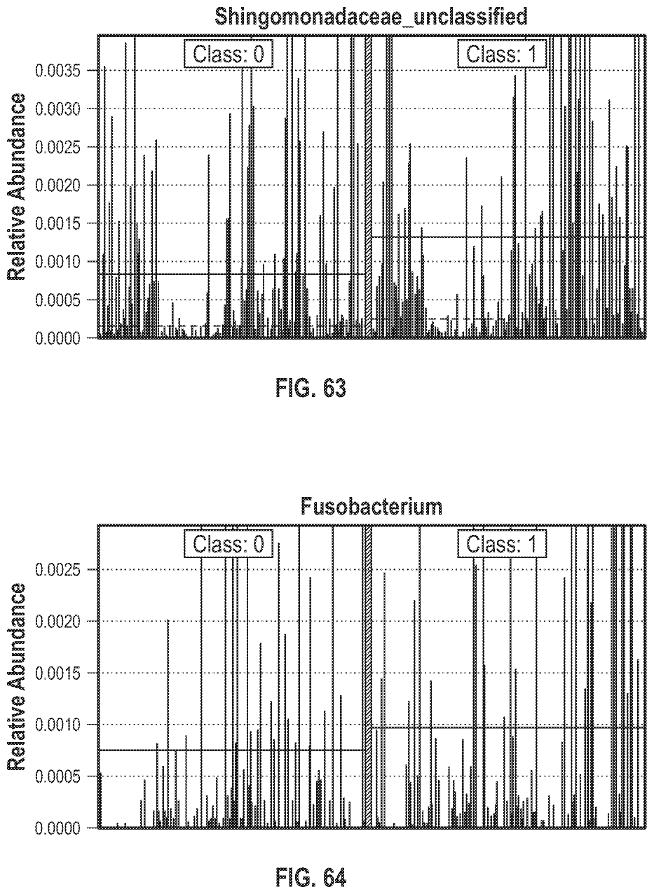

FIG. 63 is graphical representation of the significant difference in relative abundance of Sphingomonadaceae Unclassified the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Sphingomonadaceae Unclassified was significantly higher in recipients (regardless of breed type) who became pregnant after embryo transfer.

FIG. 64 is graphical representation of the significant difference in relative abundance of Fusobacterium the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Fusobacterium was significantly higher in recipients (regardless of breed type) who became pregnant after embryo transfer.

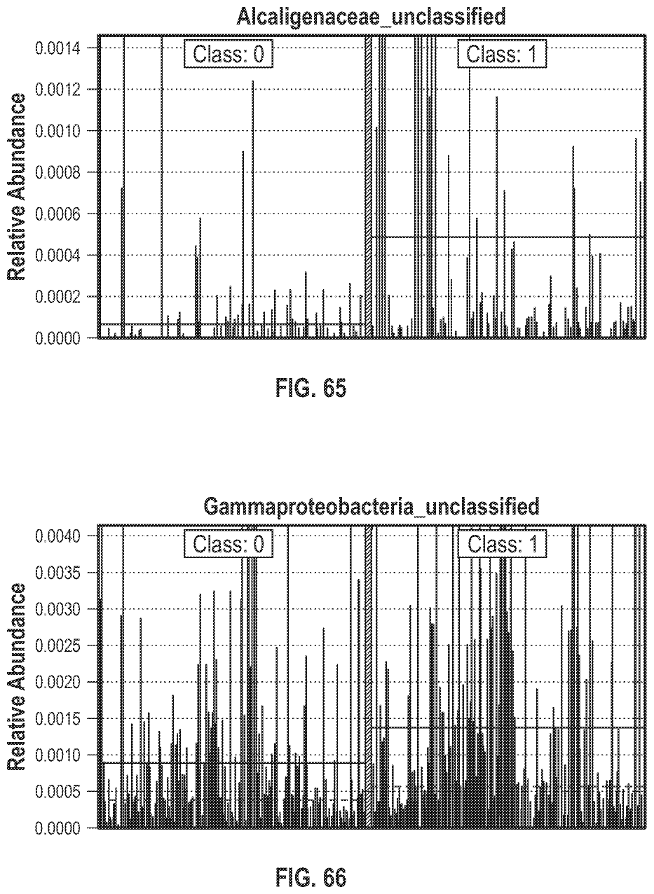

FIG. 65 is graphical representation of the significant difference in relative abundance of Alcaligenaceae Unclassified the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Alcaligenaceae Unclassified was significantly higher in recipients (regardless of breed type) who became pregnant after embryo transfer.

FIG. 66 is graphical representation of the significant difference in relative abundance of Gammaproteobacteria Unclassified the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Gammaproteobacteria Unclassified was significantly higher in recipients (regardless of breed type) who became pregnant after embryo transfer.

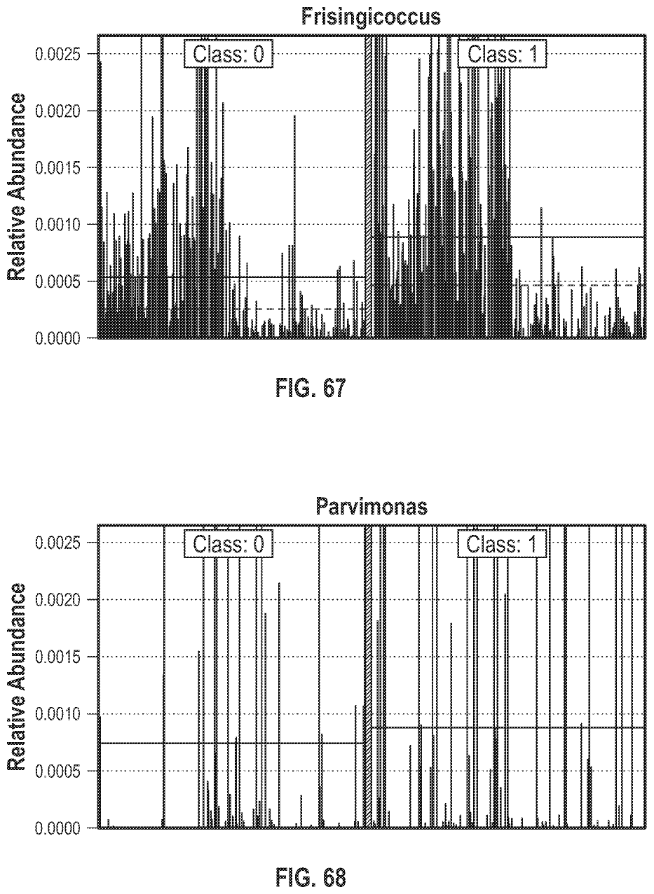

FIG. 67 is graphical representation of the significant difference in relative abundance of Frisingincoccus the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Frisingincoccus was significantly higher in recipients (regardless of breed type) who became pregnant after embryo transfer.

FIG. 68 is graphical representation of the significant difference in relative abundance of Parvimonas the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Parvimonas was significantly higher in recipients (regardless of breed type) who became pregnant after embryo transfer.

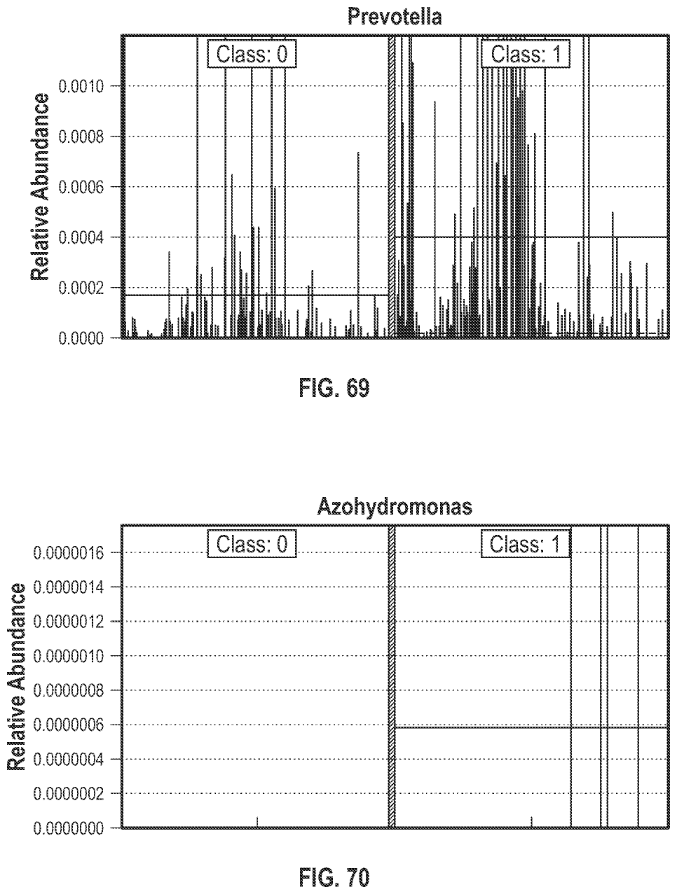

FIG. 69 is graphical representation of the significant difference in relative abundance of Prevotella the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Prevotella was significantly higher in recipients (regardless of breed type) who became pregnant after embryo transfer.

FIG. 70 is graphical representation of the significant difference in relative abundance of Azohydromonas the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Azohydromonas was significantly higher in recipients (regardless of breed type) who became pregnant after embryo transfer.

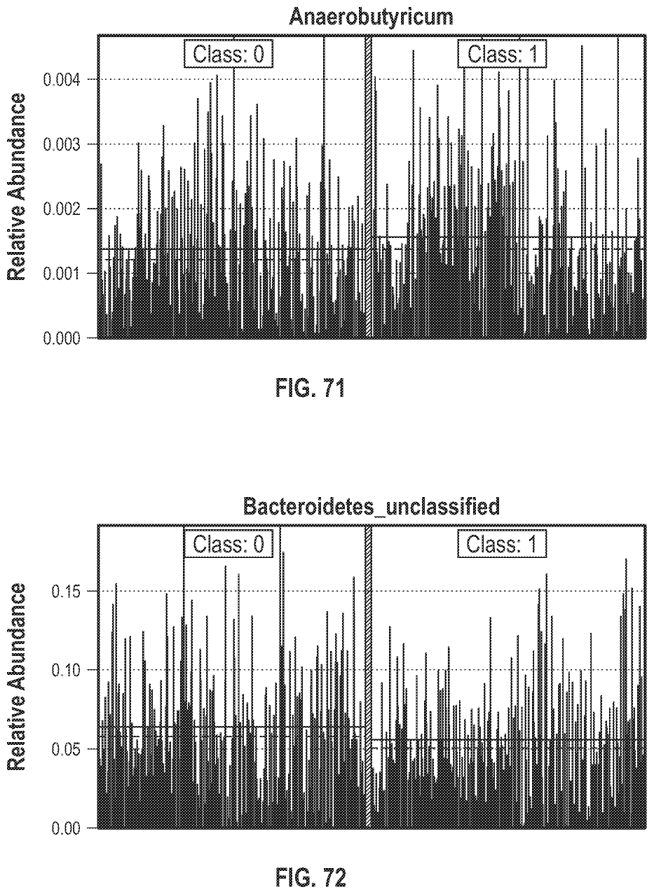

FIG. 71 is graphical representation of the significant difference in relative abundance of Anaerobutyricum the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Anaerobutyricum was significantly higher in recipients (regardless of breed type) who became pregnant after embryo transfer.

FIG. 72 is graphical representation of the significant difference in relative abundance of Bacteroidetes Unclassified the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Bacteroidetes Unclassified was significantly higher in recipients (regardless of breed type) who failed to become pregnant after embryo transfer.

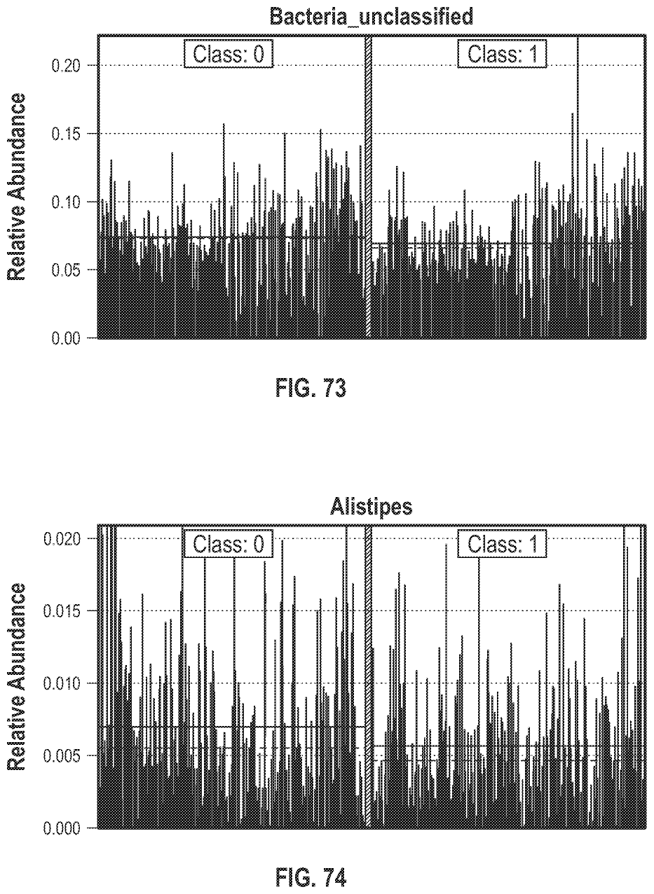

FIG. 73 is graphical representation of the significant difference in relative abundance of Bacteria Unclassified the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Bacteria Unclassified was significantly higher in recipients (regardless of breed type) who failed to become pregnant after embryo transfer.

FIG. 74 is graphical representation of the significant difference in relative abundance of Alistipes the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Alistipes was significantly higher in recipients (regardless of breed type) who failed to become pregnant after embryo transfer.

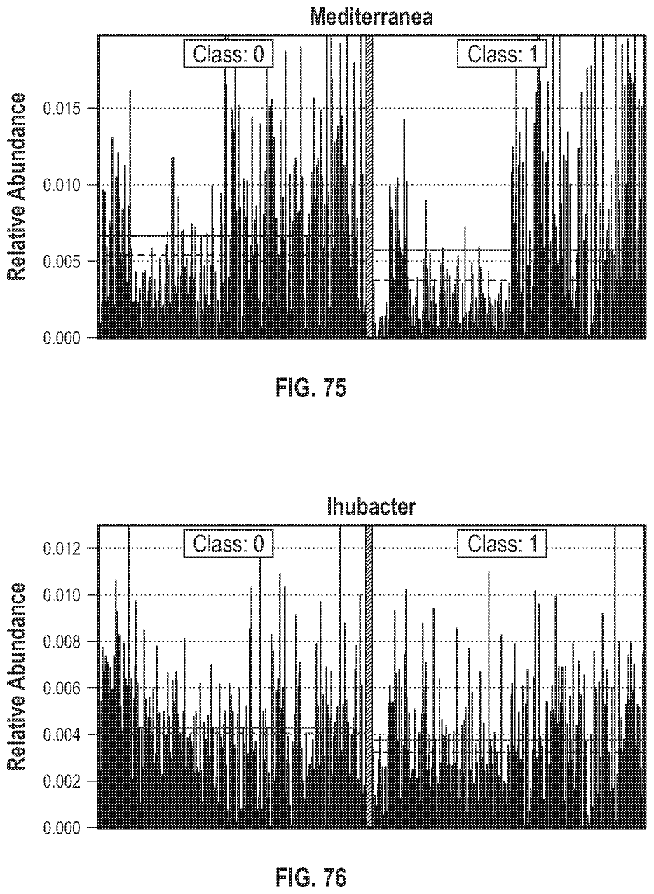

FIG. 75 is graphical representation of the significant difference in relative abundance of Mediterranea the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Mediterranea was significantly higher in recipients (regardless of breed type) who failed to become pregnant after embryo transfer.

FIG. 76 is graphical representation of the significant difference in relative abundance of Ihubacter the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Ihubacter was significantly higher in recipients (regardless of breed type) who failed to become pregnant after embryo transfer.

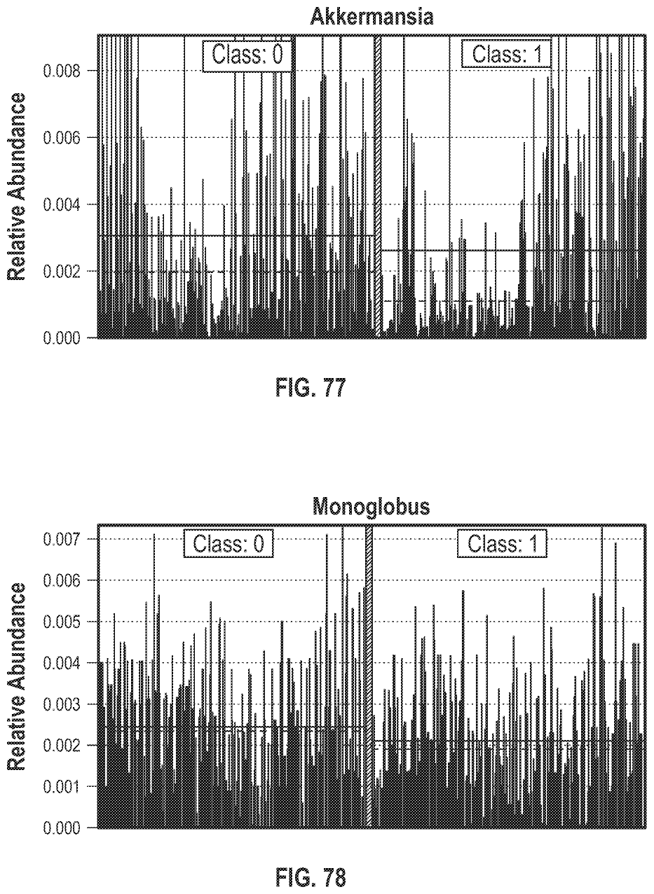

FIG. 77 is graphical representation of the significant difference in relative abundance of Akkermansia the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Akkermansia was significantly higher in recipients (regardless of breed type) who failed to become pregnant after embryo transfer.

FIG. 78 is graphical representation of the significant difference in relative abundance of Monoglobus the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Monoglobus was significantly higher in recipients (regardless of breed type) who failed to become pregnant after embryo transfer.

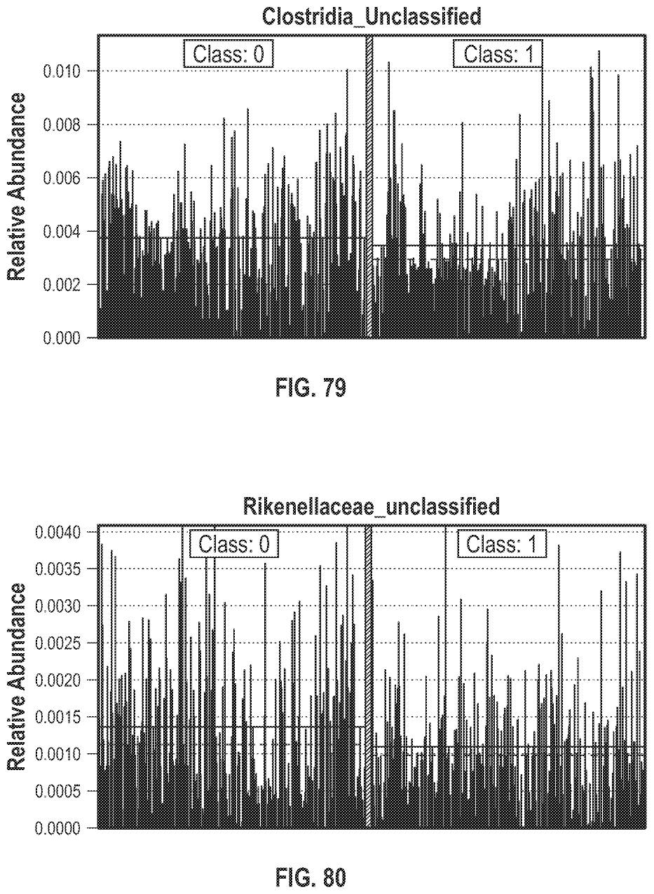

FIG. 79 is graphical representation of the significant difference in relative abundance of Clostridia Unclassified the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Clostridia Unclassified was significantly higher in recipients (regardless of breed type) who failed to become pregnant after embryo transfer.

FIG. 80 is graphical representation of the significant difference in relative abundance of Rikenellaceae Unclassified the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Rikenellaceae Unclassified was significantly higher in recipients (regardless of breed type) who failed to become pregnant after embryo transfer.

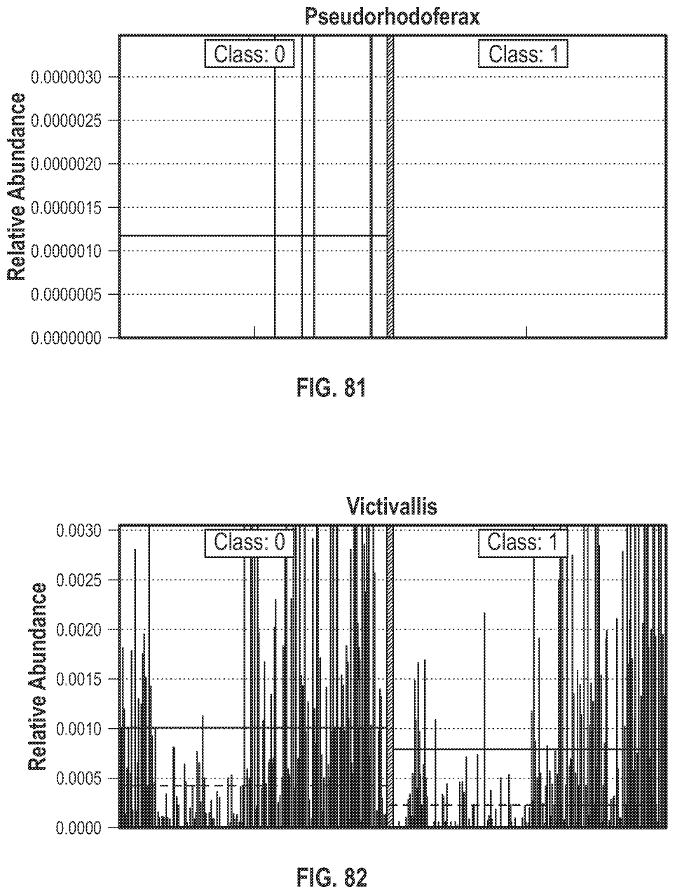

FIG. 81 is graphical representation of the significant difference in relative abundance of Pseudorhodoferax the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Pseudorhodoferax was significantly higher in recipients (regardless of breed type) who failed to become pregnant after embryo transfer.

FIG. 82 is graphical representation of the significant difference in relative abundance of Victivallis the in pregnant versus non-pregnant (regardless of breed type) recipients after embryo transfer.

The Y axis is the relative abundance, individual animals are represented by the red vertical lines.

The mean abundance by group is indicated by a solid line, the median is represented by the dashed line.

The mean relative abundance of Victivallis was significantly higher in recipients (regardless of breed type) who failed to become pregnant after embryo transfer.

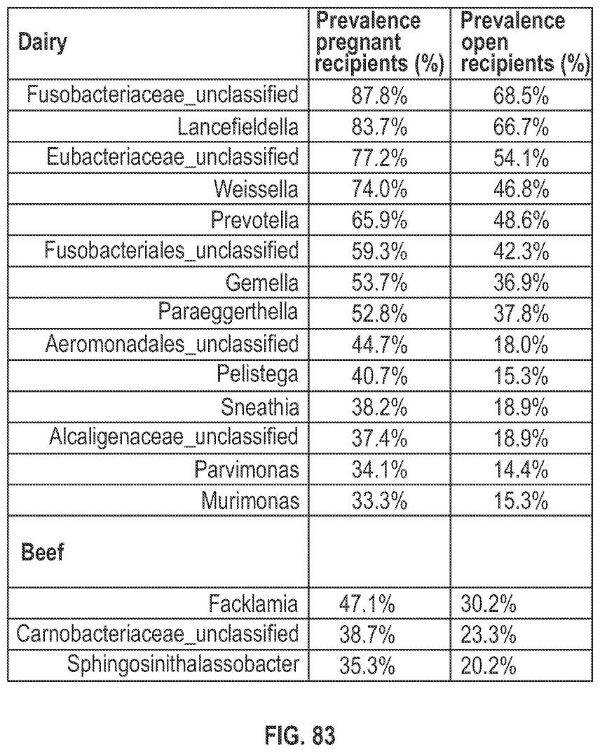

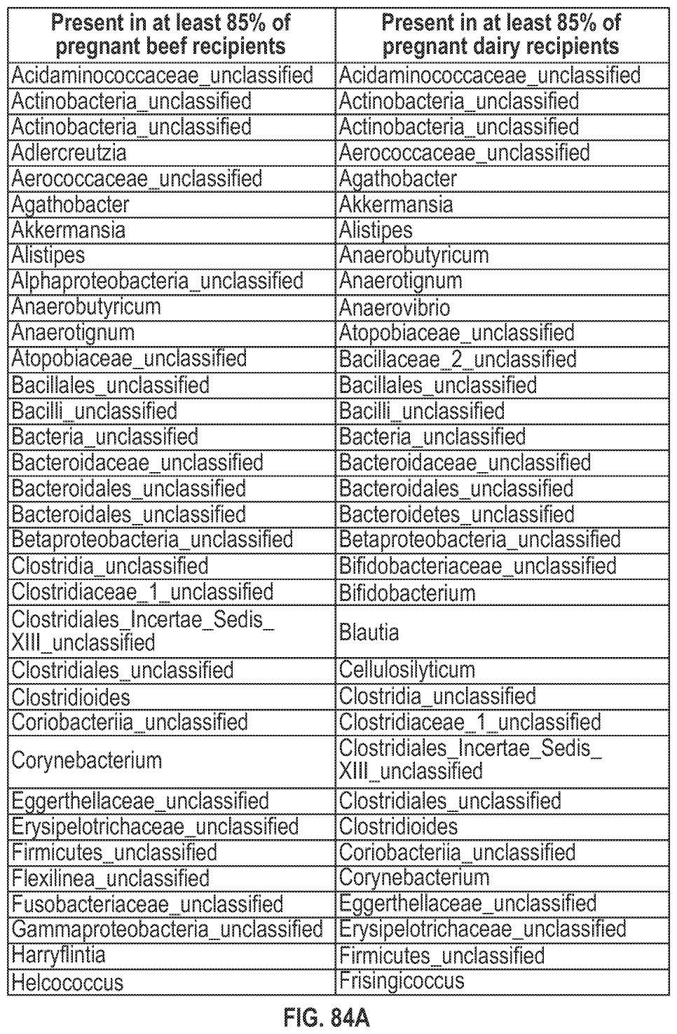

FIG. 83 is a table listing microbiota that had at least a 15% point difference in incidence (presence in particular sampled female) when comparing all pregnant versus open recipients post embryo transfer, with the higher incidence of microbiota in females that got pregnant from the embryo transfer.

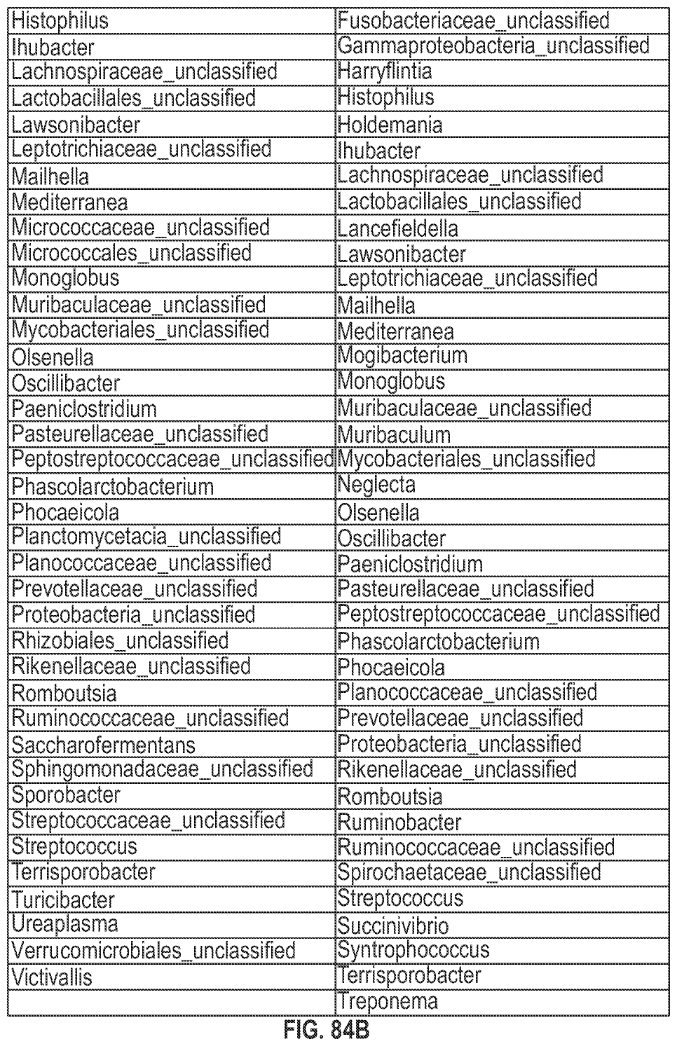

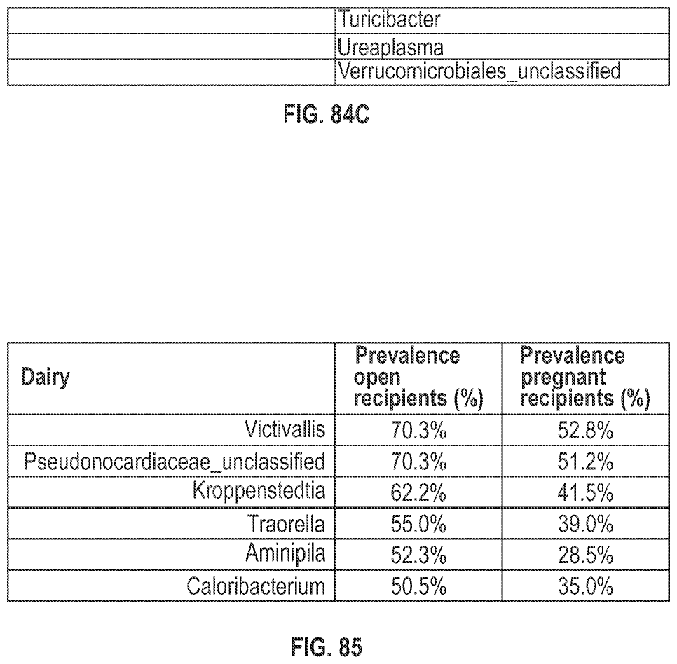

FIG. 84 is a table listing microbiota that were present in at least 85% of females that became pregnant from the embryo transfer: There were 76 microbiota that were found in at least 85% of pregnant dairy recipients, and there were 72 microbiota that were found in at least 85% of pregnant beef recipients.

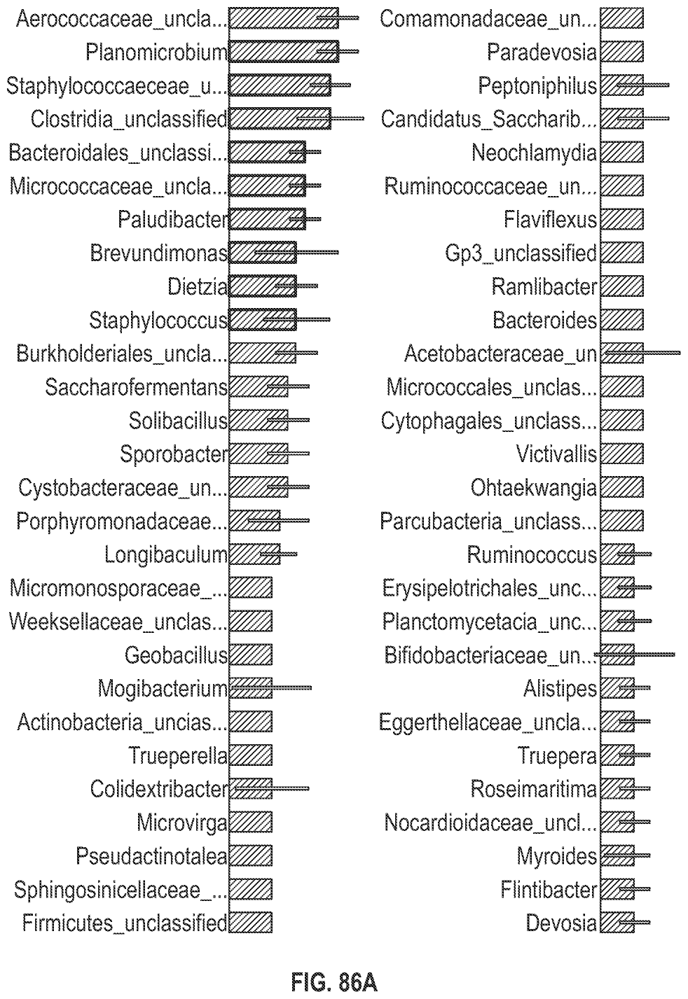

FIG. 85 is a table listing microbiota that had at least a 15% point difference in incidence (presence in particular sampled female) when comparing all pregnant versus open recipients post embryo transfer, with the higher incidence of microbiota in females that did not get pregnant from the embryo transfer.

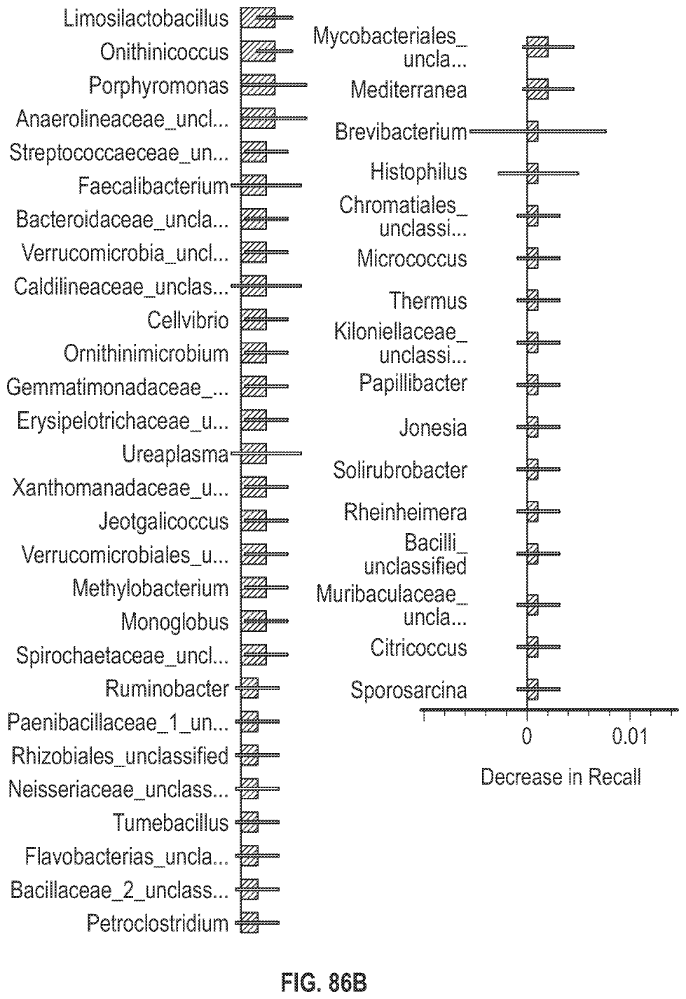

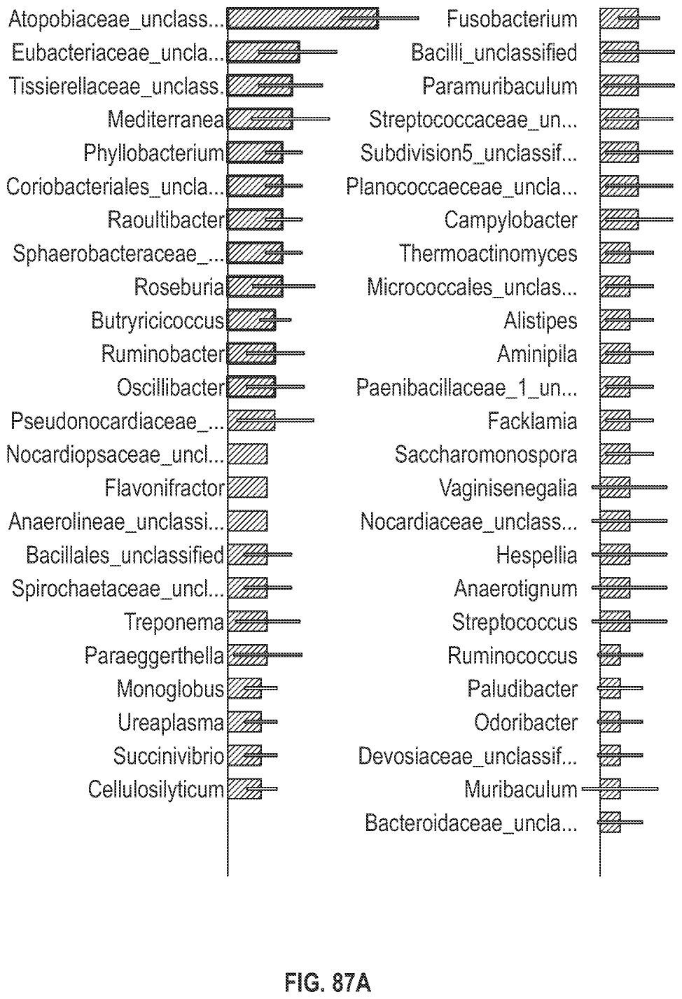

FIG. 86A random forest machine learning model was used to select for features of importance (microbiota) to predict recipient pregnancy status. Presented are the features of importance for predicting pregnancy status in Beef recipients.

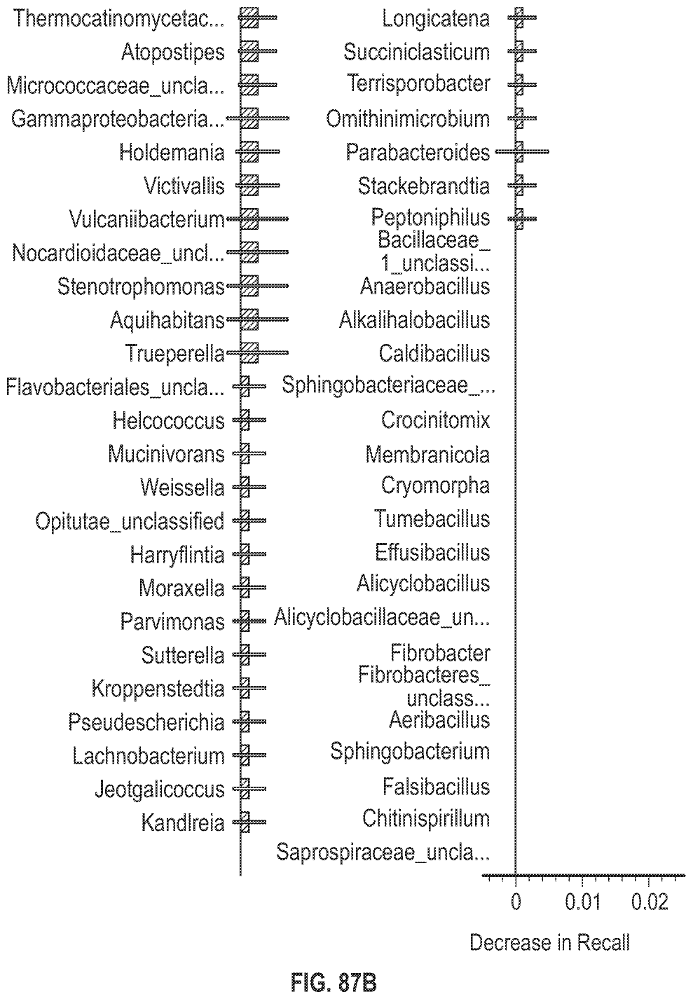

FIG. 87A random forest machine learning model was used to select for features of importance (microbiota) to predict recipient pregnancy status. Presented are the features of importance for predicting pregnancy status in Dairy recipients.

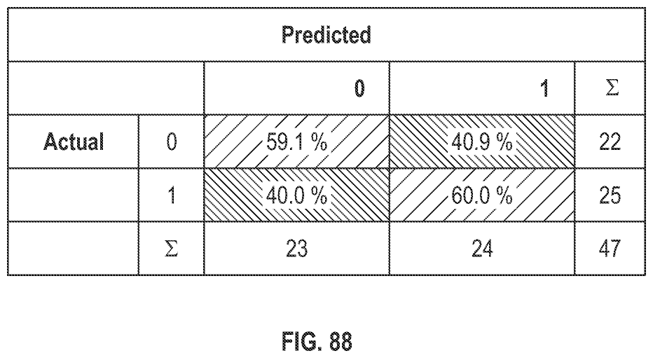

FIG. 88A random forest machine learning model was used to select for features of importance (microbiota) to predict recipient pregnancy status. Blinded samples not included in the training set were used to test the prediction models, the confusion matrix is presented showing a 60% accuracy in predicting pregnancy status post embryo transfer using selected microbiota features of importance from the model.
AUC: 0.608
CA: 0.0545
F1: 0.053
Precision: 0.557
Recall: 0.545

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. A method of increasing fertility in cattle comprising administering a microbiome supplement to the cattle comprising a combination of bacteria selected from the group consisting of Aerococcaceae_unclassified, planomicrobium, Staphylococcaceae_unclassified, Clostridia_unclassified, Bacteroidales_unclassified, Micrococcaceae_unclassified, paludibacter, brevundimonas, Dietzia, Staphylococcus, Burkholderiales_unclassified, Saccharofermentans, solibacillus, Sporobacter, cystobacteraceae_unclassified, Porphyromonadaceae_unclassified, Longibaculum, Micomonosporaceae_unclassified, Weeksellaceae_unclassified, Geobacillus, Mogibacterium, Actinobacteria_unclassified, Trueperella, Colidextribacter, microvirga, pseudactionotalea, Sphingosinicellaceae_unclassified, Firmicutes_unclassified, Comamonadaceae_unclassified, Paradevosia, Peptoniphilus, Candidatus_Solibacter_unclassified, Neochlamydia, Ruminococcaceae_unclassified, Flaviflexus, Gp3_unclassified, Ramlibacter, Bacteroides, Acetobacteraceae_unclassified, Micrococcales_unclassified, Cytophagales_unclassified, Victivallis, Ohtaekwangia, Parcubacteria_unclassified, Ruminococcus, Erysipelotrichales_unclassified, Planctomycetacia_unclassified, Bifidobacteriaceae_unclassified, Alistipes, Eggerthellaceae_unclassified, Truepera, Roseimaritima, Nocardiaceae_unclassified, Myroides, Flintibacter, Devosia, Limosilactobacillus, Ornithinibacillus, Porphyromonas, Anaerolineaceae_unclassified, Streptococcaceae_unclassified, Faecalibacterium, Bacteroidaceae_unclassified, Verrucomicrobia_unclassified, Caldilineaceae_unclassified, Cellvibrio, Ornithinimicrobium, Gemmatimonadaceae_unclassified, Erysipelotrichaceae_unclassified, Ureaplasma, Xanthomonadaceae_unclassified, Jeotgalicoccus, Verrucomicrobiales_unclassified, Methylobacterium, Monoglobus, Spirochaetaceae_unclassified, Ruminobacter, Paenibacillaceae_1_unclassified, Rhizobiales_unclassified, Neisseriaceae_unclassified, Tumebacillus, Flavobacteriales_unclassified, Bacillaceae_2_unclassified, Petroclostridium, Atopobiaceae_unclassified, Eubacteriaceae unclassified, Tissierellaceae_unclassified, Mediterranea, Phyllobacterium, Coriobacteriales_unclassified, Raoultibacter, Sphaerobacteraceae_unclassified, Roseburia, Butyricicoccus, Oscillibacter, Pseudonocardiaceae_unclassified, Nocardiopsaceae_unclassified, Flavonifractor, Anaerolineae_unclassified, Bacillales_unclassified, Treponema, Paraeggerthella, Succinivibrio, Cellulosilyticum, Fusobacterium, Bacilli_unclassified, Paramuribaculum, Planococcaceae_unclassified, Campylobacter, Thermoactinomyces, Aminipila, Facklamia, Saccharomonospora, Vaginisenegalia, Hespellia, Anaerotignum, Streptococcus, Odoribacter, Devosiaceae_unclassified, Muribaculum, Thermoactinomycetaceae_1_unclassified, Atopostipes, Gammaproteobacteria_unclassified, Holdemania, Vulcaniibacterium, Nocardioidaceae_unclassified, Stenotrophomonas, Aquihabitans, Helcococcus, Mucinivorans, Weissella, Opitutae_unclassified, Harryflintia, Moraxella, Parvimonas, Sutterella, Kroppenstedtia, Pseudescherichia, Lachnobacterium, Kandleria, Longicatena, Succiniclasticum, Terrisporobacter, Parabacteroides, Stackebrandtia, Bacillaceae_1_unclassified, Anaerobacillus, Alkalihalobacillus, Caldibacillus, Sphingobacteriaceae_unclassified, Crocinitomix, Membranicola, Cryomorpha, Effusibacillus, Alicyclobacillaceae_unclassified, Fibrobacter, Fibrobacteres_unclassified, Aeribacillus, Sphingobacterium, Falsibacillus, Chitinispirillum, Saprospiraceae_unclassified, Anaerobutyricum, Azohydromonas, Prevotella, Frisingincoccus, Gammaproteobacteria Unclassified, Alcaligenaceae Unclassified, Sphingomonadaceae Unclassified, Bifidobacteriaceae Unclassified, Bifidobacterium, Erysipelotrichaceae Unclassified, Pelistega, Fusobacteriaceae Unclassified, and Lachnospiraceae Unclassified.

39. A method of increasing fertility in cattle comprising administering a microbiome supplement to the cattle comprising a combination of bacteria selected from the group consisting of Aerococcaceae_unclassified, planomicrobium, Staphylococcaceae_unclassified, Clostridia_unclassified, Bacteroidales_unclassified, Micrococcaceae_unclassified, paludibacter, brevundimonas, Dietzia, Staphylococcus, Burkholderiales_unclassified, Saccharofermentans, solibacillus, Sporobacter, cystobacteraceae_unclassified, Porphyromonadaceae_unclassified, Longibaculum, Micomonosporaceae_unclassified, Weeksellaceae_unclassified, Geobacillus, Mogibacterium, Actinobacteria_unclassified, Trueperella, Colidextribacter, microvirga, pseudactionotalea, Sphingosinicellaceae_unclassified, Firmicutes_unclassified, Comamonadaceae_unclassified, Paradevosia, Peptoniphilus, Candidatus_Solibacter_unclassified, Neochlamydia, Ruminococcaceae_unclassified, Flaviflexus, Gp3_unclassified, Ramlibacter, Bacteroides, Acetobacteraceae_unclassified, Micrococcales_unclassified, Cytophagales_unclassified, Victivallis, Ohtaekwangia, Parcubacteria_unclassified, Ruminococcus, Erysipelotrichales_unclassified, Planctomycetacia_unclassified, Bifidobacteriaceae_unclassified, Alistipes, Eggerthellaceae_unclassified, Truepera, Roseimaritima, Nocardiaceae_unclassified, Myroides, Flintibacter, Devosia, Limosilactobacillus, Ornithinibacillus, Porphyromonas, Anaerolineaceae_unclassified, Streptococcaceae_unclassified, Faecalibacterium, Bacteroidaceae_unclassified, Verrucomicrobia_unclassified, Caldilineaceae_unclassified, Cellvibrio, Ornithinimicrobium, Gemmatimonadaceae_unclassified, Erysipelotrichaceae_unclassified, Ureaplasma, Xanthomonadaceae_unclassified, Jeotgalicoccus, Verrucomicrobiales_unclassified, Methylobacterium, Monoglobus, Spirochaetaceae_unclassified, Ruminobacter, Paenibacillaceae_1_unclassified, Rhizobiales_unclassified, Neisseriaceae_unclassified, Tumebacillus, Flavobacteriales_unclassified, Bacillaceae_2_unclassified, and Petroclostridium, Corynebacterium, Streptococcus, Staphylococcaceae_unclassified, Bogoriellaceae unclassified, Saccharomonospora, Bdellovibrio, Brevibacterium, Carnobacteriaceae unclassified, Succiniclasticum, Actinobacteria unclassified, Sneathia, Staphylococcaceae unclassified, Sneathia, Corynebacterium and Sphingomonadaceae unclassified.

40. A method of increasing fertility in cattle comprising administering a microbiome supplement to the cattle comprising a combination of bacteria selected from the group consisting of Atopobiaceae_unclassified, Eubacteriaceae_unclassified, Tissierellaceae_unclassified, Mediterranea, Phyllobacterium, Coriobacteriales_unclassified, Raoultibacter, Sphaerobacteraceae_unclassified, Roseburia, Butyricicoccus, Ruminobacter, Oscillibacter, Pseudonocardiaceae_unclassified, Nocardiopsaceae_unclassified, Flavonifractor, Anaerolineae_unclassified, Bacillales_unclassified, Spirochaetaceae_unclassified, Treponema, Paraeggerthella, Monoglobus, Ureaplasma, Succinivibrio, Cellulosilyticum, Fusobacterium, Bacilli_unclassified, Paramuribaculum, Streptococcaceae_unclassified, Subdivision5_unclassified, Planococcaceae_unclassified, Campylobacter, Thermoactinomyces, Micrococcales_unclassified, Alistipes, Aminipila, Paenibacillaceae_1_unclassified, Facklamia, Saccharomonospora, Vaginisenegalia, Nocardiaceae_unclassified, Hespellia, Anaerotignum, Streptococcus, Ruminococcus, Paludibacter, Odoribacter, Devosiaceae_unclassified, Muribaculum, Bacteroidaceae_unclassified, Thermoactinomycetaceae_1_unclassified, Atopostipes, Micrococcaceae_unclassified, Gammaproteobacteria_unclassified, Holdemania, Victivallis, Vulcaniibacterium, Nocardioidaceae_unclassified, Stenotrophomonas, Aquihabitans, Trueperella, Flavobacteriales_unclassified, Helcococcus, Mucinivorans, Weissella, Opitutae_unclassified, Harryflintia, Moraxella, Parvimonas, Sutterella, Kroppenstedtia, Pseudescherichia, Lachnobacterium, Jeotgalicoccus, Kandleria, Longicatena, Succiniclasticum, Terrisporobacter, Ornithinimicrobium, Parabacteroides, Stackebrandtia, Peptoniphilus, Bacillaceae_1_unclassified, Anaerobacillus, Alkalihalobacillus, Caldibacillus, Sphingobacteriaceae_unclassified, Crocinitomix, Membranicola, Cryomorpha, Tumebacillus, Effusibacillus, Alicyclobacillaceae_unclassified, Fibrobacter, Fibrobacteres_unclassified, Aeribacillus, Sphingobacterium, Falsibacillus, Chitinispirillum, Saprospiraceae_unclassified, Lachnospiraceae unclassified, Histophilus, Stomatobactulum, Rothia, Bifidobacteriaceae unclassified, Quasibacillus, Limosilactobacillus, Fusicatenibacter, Anaeroplasma, Mogibacterium, Neisseriaceae unclassified, Fusobacteriacea unclassified, Canibacter, Erysipelotrichaceae unclassified, Pelistega, Gemella, Gammaproteobacteria unclassified, Bifidobacterium, Selenomonadaceae unclassified, Prevotellaceae unclassified, Alcaligenaceae unclassified, Salinicoccus, Gp16 unclassified, Camelimonas, Schwartzia, Aeromucrobium, Marvinbryantia, Desulfovibrianaceae unclassified, Pasteurellaceae unclassified, Mycobacteriales unclassified, Micrococcaceae unclassified, Coriobacteriia unclassified, Frisingicoccus, Anaerobutyricum, Marinitenerispora, Agathobacter, Ligilactobacillus, Ligilactobacillus, Ligilactobacullus, Olsenella, Prevotella, Coriobacteria, Gammaproteabacteria unclassified, Alcaligenaceae, Bifidobacterium, Erysipelotricheae, Fusobacteriaceae, and Lachnospiraceae unclassified.

41. The supplement of claim 38, 39, or 40 wherein the supplement is administered orally.

42. The supplement of claim 38, 39, or 40 wherein the supplement is administered vaginally.

43. The method of claim 38 or 39 wherein the breed of a parent of said cattle is selected from the group consisting of Angus, Hereford, Simmental, Charolais, Red Angus, or Brahman.

44. The methods of claim 38 or 40 wherein the breed of a parent of said cattle is selected from the group consisting of Holstein, Jersey, Swedish Red, or Mt. Boulliard.

45. A method of increasing fertility in cattle comprising:
(a) administering a microbiome supplement comprising a combination of bacteria selected from the group consisting of Aerococcaceae_unclassified, planomicrobium, Staphylococcaceae_unclassified, Clostridia_unclassified, Bacteroidales_unclassified, Micrococcaceae_unclassified, paludibacter, brevundimonas, Dietzia, Staphylococcus, Burkholderiales_unclassified, Saccharofermentans, solibacillus, Sporobacter, cystobacteraceae_unclassified, Porphyromonadaceae_unclassified, Longibaculum, Micomonosporaceae_unclassified, Weeksellaceae_unclassified, Geobacillus, Mogibacterium, Actinobacteria_unclassified, Trueperella, Colidextribacter, microvirga, pseudactionotalea, Sphingosinicellaceae_unclassified, Firmicutes_unclassified, Comamonadaceae_unclassified, Paradevosia, Peptoniphilus, Candidatus_Solibacter_unclassified, Neochlamydia, Ruminococcaceae_unclassified, Flaviflexus, Gp3_unclassified, Ramlibacter, Bacteroides, Acetobacteraceae_unclassified, Micrococcales_unclassified, Cytophagales_unclassified, Victivallis, Ohtaekwangia, Parcubacteria_unclassified, Ruminococcus, Erysipelotrichales_unclassified, Planctomycetacia_unclassified, Bifidobacteriaceae_unclassified, Alistipes, Eggerthellaceae_unclassified, Truepera, Roseimaritima, Nocardiaceae_unclassified, Myroides, Flintibacter, Devosia, Limosilactobacillus, Ornithinibacillus, Porphyromonas, Anaerolineaceae_unclassified, Streptococcaceae_unclassified, Faecalibacterium, Bacteroidaceae_unclassified, Verrucomicrobia_unclassified, Caldilineaceae_unclassified, Cellvibrio, Ornithinimicrobium, Gemmatimonadaceae_unclassified, Erysipelotrichaceae_unclassified, Ureaplasma, Xanthomonadaceae_unclassified, Jeotgalicoccus, Verrucomicrobiales_unclassified, Methylobacterium, Monoglobus, Spirochaetaceae_unclassified, Ruminobacter, Paenibacillaceae_1_unclassified, Rhizobiales_unclassified, Neisseriaceae_unclassified, Tumebacillus, Flavobacteriales_unclassified, Bacillaceae_2_unclassified, Petroclostridium, Atopobiaceae_unclassified, Eubacteriaceae_unclassified, Tissierellaceae_unclassified, Mediterranea, Phyllobacterium, Coriobacteriales_unclassified, Raoultibacter, Sphaerobacteraceae_unclassified, Roseburia, Butyricicoccus, Oscillibacter, Pseudonocardiaceae_unclassified, Nocardiopsaceae_unclassified, Flavonifractor, Anaerolineae_unclassified, Bacillales_unclassified, Treponema, Paraeggerthella, Succinivibrio, Cellulosilyticum, Fusobacterium, Bacilli_unclassified, Paramuribaculum, Planococcaceae_unclassified, Campylobacter, Thermoactinomyces, Aminipila, Facklamia, Saccharomonospora, Vaginisenegalia, Hespellia, Anaerotignum, Streptococcus, Odoribacter, Devosiaceae_unclassified, Muribaculum, Thermoactinomycetaceae_1_unclassified, Atopostipes, Gammaproteobacteria_unclassified, Holdemania, Vulcaniibacterium, Nocardioidaceae_unclassified, Stenotrophomonas, Aquihabitans, Helcococcus, Mucinivorans, Weissella, Opitutae_unclassified, Harryflintia, Moraxella, Parvimonas, Sutterella, Kroppenstedtia, Pseudescherichia, Lachnobacterium, Kandleria, Longicatena, Succiniclasticum, Terrisporobacter, Parabacteroides, Stackebrandtia, Bacillaceae_1_unclassified, Anaerobacillus, Alkalihalobacillus, Caldibacillus, Sphingobacteriaceae_unclassified, Crocinitomix, Membranicola, Cryomorpha, Effusibacillus, Alicyclobacillaceae_unclassified, Fibrobacter, Fibrobacteres_unclassified, Aeribacillus, Sphingobacterium, Falsibacillus, Chitinispirillum, Saprospiraceae_unclassified, Anaerobutyricum, Azohydromonas, Prevotella, Frisingincoccus, Gammaproteobacteria Unclassified, Alcaligenaceae Unclassified, Sphingomonadaceae Unclassified, Bifidobacteriaceae Unclassified, Bifidobacterium, Erysipelotrichaceae Unclassified, Pelistega, Fusobacteriaceae Unclassified, and Lachnospiraceae Unclassified to the cattle,
(b) measuring the level of fertility in the animal before and after administering the supplement, and;
(c) determining an increase in fertility in the animal as a result of administering the supplement.

46. A method for improving fertility in one or more bovine animals, the method comprising: (a) collecting vaginal fluid from each of the bovine animals, wherein the vaginal fluid comprises a microbiome of each of the bovine animal; (b) performing microbiome analysis on the vaginal fluids to obtain a vaginal microbiome profile, wherein the microbiome composition is predictive of an increased fertility; and (c) administering an intervention to one or more of the bovine dams, wherein the intervention is effective to provide a bovine animal with a microbiome profile associated with an increased fertility.

47. A diagnostic test kit for identifying a bovine animal having increased fertility comprising: (a) one or more analytical tools for determining a vaginal microbiome profile comprising bacterial species from the bovine animal; (b) a transmitter to communicate/connect a database of one or more microbiome components of the vaginal fluid; and (c) a device for comparing the vaginal microbiome from the bovine animal with a database of vaginal microbiome which identifies a bovine animal having a vaginal microbiome that is predictive of increased fertility.

48. A diagnostic test kit for identifying a bovine animal having decreased fertility comprising: (a) one or more analytical tools for determining a vaginal microbiome profile comprising bacterial species from the bovine animal; (b) a transmitter to communicate/connect a database of one or more microbiome components of the vaginal fluid; and (c) a device for comparing the vaginal microbiome from the bovine animal with a database of vaginal microbiome which identifies a bovine animal having a vaginal microbiome that is predictive of decreased fertility.

49. A method of increasing fertility in cattle comprising administering an antimicrobial to the cattle targeted to bacteria selected from the group comprising Falcatimonas, Dorea, Pelagibacterium, Lawsonibacterium, Clostridium IV, Planococcaceae unclassified, Sporobacter, Rhodothermales unclassified, Vulcaniibacterium, Rikenellaceae unclassified, Alkalibacterium, Harryfintia, Verrucomicrobiales unclassified, Ihubacter, Oscillibacter, Victivallis, Actinomarinicola, Akkermansia, Alistipes, Bacteroidaceae unclassified, Bacteroides unclassified, Bacillales unclassified, Streptosporangiaceae unclassified, Mediterranea, Lactobacillus unclassified, Turicibacter, Hazenalla, Tissierella, Deinococci unclassified, Gulosibacter, Schaedlerella, Psychrobacillus, Irregularibacter, Cumulibacter, Anaerotignum, Cohnella, Clostridia unclassified, Muribaculaceae unclassified, Firmicutes unclassified, Rumunococcaceae unclassified, Arcanobacterium, Flintibacter, Leptotrichia, Lactobacillus, Patulibacter, Pseudarthrobacter, Acidbacter unclassified, Sandaracinus, Histophilus, Falcatimonas, Anaerobacter, Nocardiopsis, Vulgatibacter, Micrococcales unclassified, Succinivibrio, Fusobacteriales unclassified, Treponema, Histophilus, Pseudorhodoferax, Monoglobus, Bacteroidetes Unclassified, and/or Bacteroidetes Unclassified.

50. A method of increasing fertility in beef cattle, comprising administering an antimicrobial to the beef cattle targeted to bacteria selected from the group comprising Arcanobacterium, Flintibacter, Leptotrichia, Succinivibrio, Fusobacteriales inclassified, Treponema, Lactobacillus, Patulibacter, Pseudarthrobacter, Acidbacter unclassified, Sandaracinus, Histophilus and Histophilus.

51. A method of increasing fertility in dairy cattle, comprising administering an antimicrobial to the dairy cattle targeted to bacteria selected from the group comprising Falcatimonas, Bacteroidetes Unclassified, Bacteria Unclassified, Ruminococcaceae Unclassified, Dorea, Pelagibacterium, Lawsonibacterium, Clostridium IV, Planococcaceae unclassified, Sporobacter, Rhodothermales unclassified, Vulcaniibacterium, Rikenellaceae unclassified, Anaerobacter, Alkalibacterium, Harryfintia, Nocardiopsis, Actinomarinicola, Bacteroidaceae unclassified, Bacillales unclassified, Streptosporangiaceae unclassified, Lactobacillies unclassified, Turicibacter, Hazenalla, Tissierella, Roseburia, Deinococci unclassified, Gulosibacter, Schaedlerella, Rumunococcaceae unclassified, Rikenellaceae unclassified, Verrucomicrobiales unclassified, Ihubacter, Oscillibacter, Victivallis, Akkermansia, Alistipes, Bacteroides unclassified, Mediterranea, Vulgatibacter, Irregularibacter, Cumulibacter, Micrococcales unclassified, Anaerotignum, Cohnella, Clostridia unclassified, Muribaculaceae unclassified, Firmicutes unclassified, Monoglobus, Rumunococcaceae unclassified and Psychrobacillus.

52. The supplement of claim 49, 50, or 51, wherein the supplement is administered orally.

53. The supplement of claim 49, 50, or 51 wherein the supplement is administered vaginally.
